(19) United States
(12) Patent Application Publication
(10) Pub. No.: US 2024/0115730 A1
(43) Pub. Date: Apr. 11, 2024

(54) TISSUE-SPECIFIC NUCLEIC ACID DELIVERY BY 1,2-DIOLEOYL-3-TRIMETHYLAMMONIUM-PROPANE (DOTAP) LIPID NANOPARTICLES

(71) Applicant: OMEGA THERAPEUTICS, INC., Cambridge, MA (US)

(72) Inventors: Vishwesh Ashok Patil, Arlington, MA (US); Can Sarisozen, Westford, MA (US); Marcus Ian Gibson, Belmont, MA (US); Daniel Ferreira Gomes Costa, Cambridge, MA (US)

(73) Assignee: OMEGA THERAPEUTICS, INC., Cambridge, MA (US)

(21) Appl. No.: 18/255,906

(22) PCT Filed: Dec. 15, 2021
(86) PCT No.: PCT/US2021/063555

§ 371 Date: Jun. 5, 2023

Related U.S. Application Data
(60) Provisional application No. 63/127,812, filed on Dec. 18, 2020.

(51) Int. Cl. A61K 47/69; A61K 47/54; A61K 47/60

(52) U.S. Cl. CPC A61K 47/6929; A61K 47/543; A61K 47/60

(57) ABSTRACT

The instant disclosure relates to nucleic acid-lipid particles having 1,2-dioleoyl-3-trimethylammonium-propane (DOTAP), which preferentially localize and deliver associated cargoes to the lung and various lung tissues, as well as to tissues to which such particles are directly injected. The instant disclosure provides compositions comprising such lipid particles, optionally in association with a therapeutic agent (e.g., a therapeutic mRNA and/or nucleic acid controller system), as well as methods and kits for delivering a lipid particle-associated therapeutic agent and/or treating a disease or disorder, e.g., a lung disease or disorder, in a subject, using the lipid particle compositions provided herein.

### CROSS-REFERENCE TO RELATED APPLICATION

The present application is related to and claims priority under 35 U.S.C. § 119(e) to U.S. provisional patent application No. 63/127,812, entitled “Tissue-Specific Nucleic Acid Delivery By 1,2-Dioleoyl-3-Trimethylammonium-Propane (DOTAP) Lipid Nanoparticles,” filed Dec. 18, 2020. The entire content of the aforementioned patent application is incorporated herein by this reference.

### FIELD OF THE INVENTION

The current disclosure relates to lipid-based compositions and methods useful in administering nucleic acid-based therapies. In particular, the disclosure relates to 1,2-dioleoyl-3-trimethylammonium-propane (DOTAP) lipid compositions for treating diseases and disorders in tissues of a subject, including lung tissues of a subject.

### BACKGROUND OF THE INVENTION

The World Health Organization reports that lung diseases are the leading cause of death and disability in the world. Lung disease and other breathing problems, such as newborn respiratory distress syndrome, constitute one of the leading causes of death in babies less than one year old. About 65 million people suffer from chronic obstructive pulmonary disease (COPD) alone, and 3 million die from it each year (www.who.int/news-room/fact-sheets/detail/chronic-obstructive-pulmonary-disease-(copd)). Although some treatments exist for these conditions, they are by no means completely restorative, and a major challenge in the field of medicine remains to develop therapeutic agents that effectively treat diseases without prohibitively harming the patient.

Nucleic acid therapies offer tremendous potential for treatment of diseases at the level of individual, targeted genes. However, safe and effective delivery systems are essential for realizing the full promise of nucleic acid therapeutics. Non-specific delivery of nucleic acid therapeutics to all organs and tissues can often result in off-site (non-targeted and/or off-target) effects and toxicity. Delivery of nucleic acid therapeutics preferentially to an organ or tissue of interest in which a specific action is desirable is a continuing goal for drug delivery and delivery of nucleic acid-based agents in particular. The concept of only targeting the cause of a disease without harming other parts of the body was described by Ehrlich 120 years ago. However, there are still effectively no options for nanoparticle delivery systems that are capable of targeting specific tissues without introducing ligand-based targeting strategies (with the latter also referred to as active targeting). There is therefore a previously unmet need in the art for delivery modalities that are capable of achieving organ-specific delivery of nucleic acid cargoes based only upon the structural components of such delivery modalities (rather than by ligand-based active targeting strategies). In particular, because lungs are a key target organ for gene therapy, there is also a specific need in the art for such delivery modalities capable of selectively delivering nucleic acid cargoes to the lungs.

### BRIEF SUMMARY OF THE INVENTION

The instant disclosure is based, at least in part, upon identification of lipid-based nanoparticle compositions and formulations capable of specifically targeting a cargo moiety (e.g., a nucleic acid cargo) to the lung and lung tissues of a subject, without requiring a ligand-based targeting strategy. DOTAP, a well-known quaternary amino lipid, is a structural component of the lipid nanoparticles (LNPs) of the instant disclosure that, upon systemic or local administration, has been remarkably identified herein to shift the tropism of vectors specifically to lungs without requiring a further active-targeting component in the LNPs. The instant disclosure indicates the surprising structural affinity DOTAP possesses for lung tissues, which can be exploited for effective delivery of nucleic acid cargoes, including, e.g., expression of therapeutic mRNAs, upon systemic administration (e.g., via intravenous (IV) injection). While fluorescently-labeled DOTAP LNPs of the instant disclosure were identified herein to accumulate in the liver at levels of up to 25-40% of total LNP, significantly preferential expression of LNP-borne cargo mRNA (activity) was observed in the lungs as compared to liver and all other tissues examined. Such observed effects were independent of the magnitude of the surface charge of tested LNP formulations. Immunohistochemistry (IHC) evaluation of lung tissues also demonstrated successful delivery and expression of cargo mRNA in endothelial cells, epithelial cells, fibroblasts and macrophages using the DOTAP LNPs disclosed herein. Moreover, it was also identified herein that lung-delivering DOTAP-based LNPs can be prepared advantageously without PEG in the formulation. Without wishing to be bound by theory, the high positive charge of DOTAP appears to be sufficient to stabilize the particles of the instant disclosure via electrostatic stabilization, without requiring steric stabilization. The ability to exclude PEG from certain highly active lipid particle formulations disclosed herein is another notable and surprising feature of the particles of the instant disclosure. Indeed, without wishing to be bound by theory, it is believed that employment of PEG-free compositions can reduce or even entirely avoid the accelerated blood clearance (ABC) effect previously described for PEG-containing LNPs, which is a well-documented phenomenon caused by a subject's immune system activating against PEG molecules on the surface of LNPs. ABC is responsible for the clearance of nanoparticles from systemic circulation upon repeated dosing. The instant disclosure, therefore, significantly provides nucleic acid-lipid particles that offer particular advantages for repeated systemic administration, as the LNPs of the instant disclosure integrated and employ DOTAP as a stabilizing lipid.

In one aspect, the instant disclosure provides a nucleic acid-lipid particle for delivering a nucleic acid cargo to a lung tissue of a subject, the nucleic acid-lipid particle including 1,2-dioleoyl-3-trimethylammonium-propane (DOTAP) at a concentration from 20 mol % to 80 mol % of the total lipid present in the nucleic acid-lipid particle.

In certain embodiments, the nucleic acid-lipid particle includes a conjugated lipid that inhibits aggregation of particles present at a concentration from 0.01 to 2% of the total lipid present. Optionally, the conjugated lipid includes a polyethyleneglycol (PEG)-lipid conjugate. Optionally, the PEG of the PEG-lipid conjugate has an average molecular weight of from 550 daltons to 3000 daltons. Optionally, the PEG-lipid conjugate is a PEG2000-lipid conjugate. Optionally, the PEG2000-lipid conjugate includes one or more of 1,2-dimyristoyl-rac-glycero-3-methoxypolyethylene glycol-2000 (DMG-PEG2k) and 1,2-distearoyl-rac-glycero-3-methoxypolyethylene glycol-2000 (DSG-PEG2k). Optionally, the PEG2000-lipid conjugate is 1,2-Dimyristoyl-rac-glycero-3-methoxypolyethylene glycol-2000 (DMG-PEG2k). Optionally, the nucleic acid-lipid particle includes a PEG-lipid conjugate at a concentration of about 0.5 mol % of the total lipid present in the nucleic acid-lipid particle, of about 1.0 mol % of the total lipid present in the nucleic acid-lipid particle, or of about 1.5 mol % of the total lipid present in the nucleic acid-lipid particle.

In some embodiments, the nucleic acid-lipid particle does not include a PEG-lipid conjugate. Optionally, the nucleic acid-lipid particle does not include PEG. Optionally, the nucleic acid-lipid particle is a component of a multi-dose therapy.

In one embodiment, the nucleic acid-lipid particle includes one or more non-cationic lipids at a concentration of from 20 mol % to 80 mol % of the total lipid present in the lipid-nucleic acid particle. Optionally, the one or more non-cationic lipids include cholesterol or a derivative thereof.

In related embodiments, the nucleic acid-lipid particle includes cholesterol or a derivative thereof at one of the following concentration ranges: 10 mol % to 20 mol % of the total lipid present in the nucleic acid-lipid particle; 35 mol % to 45 mol % of the total lipid present in the nucleic acid-lipid particle; and 60 mol % to 70 mol % of the total lipid present in the nucleic acid-lipid particle.

In certain embodiments, the nucleic acid-lipid particle includes one or more non-cationic lipid other than cholesterol or a derivative thereof. Optionally, the one or more non-cationic lipid other than cholesterol or a derivative thereof is present at from 5 mol % to 20 mol % of the total lipid present in the lipid-nucleic acid particle. Optionally, the one or more non-cationic lipid other than cholesterol or a derivative thereof is present at about 10 mol % of the total lipid present in the nucleic acid-lipid particle. Optionally, the one or more non-cationic lipid other than cholesterol or a derivative thereof includes 1,2-dioleoyl-sn-glycero-3-phosphocholine (DOPC), 1,2-Distearoyl-sn-glycero-3-phosphocholine (DSPC), 1,2-Dioleoyl-sn-glycero-3-phosphoethanolamine (DOPE) and/or β-sitosterol. Optionally, the one or more non-cationic lipid other than cholesterol or a derivative thereof is dioleoylphosphatidylcholine (DOPC).

In embodiments, the nucleic acid cargo includes a synthetic or naturally occurring RNA or DNA, or derivatives thereof. Optionally, the nucleic acid cargo is a modified RNA. Optionally, the modified RNA is a modified mRNA, a modified antisense oligonucleotide or a modified siRNA. Optionally, the modified mRNA encodes a nucleic acid modulating controller.

In certain embodiments, the nucleic acid cargo includes one or more of the following modifications: 2′-O-methyl modified nucleotides, a nucleotide including a 5′-phosphorothioate group, a terminal nucleotide linked to a cholesteryl derivative, a 2′-deoxy-2′-fluoro modified nucleotide, a 5′-methoxy-modified nucleotide (e.g., 5′-methoxyuridine), a 2′-deoxy-modified nucleotide, a locked nucleotide, an abasic nucleotide, a 2′-amino-modified nucleotide, a 2′-alkyl-modified nucleotide, a morpholino nucleotide, a phosphoramidate, a non-natural base including nucleotide; internucleoside linkages or backbones including phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and/or those having inverted polarity wherein the adjacent pairs of nucleoside units are linked 3′-5′ to 5′-3′ or 2′-5′ to 5′-2′.

In embodiments, the lung tissue is one or more of: epithelium, endothelium, interstitial connective tissue, blood vessel, hematopoietic tissue, lymphoid tissue and pleura.

In some embodiments, the nucleic acid-lipid particle includes DOTAP at from 20 mol % to 49 mol % of the total lipid present in the nucleic acid-lipid particle. Optionally, the nucleic acid-lipid particle includes DOTAP at about 25 mol % or about 45 mol % of the total lipid present in the nucleic acid-lipid particle.

In certain embodiments, the nucleic acid-lipid particle includes DOTAP at about 50 mol % or about 75 mol % of the total lipid present in the nucleic acid-lipid particle.

Another aspect of the instant disclosure provides a pharmaceutical composition that includes a nucleic acid-lipid particle of the disclosure and a pharmaceutically acceptable carrier.

In embodiments, the pharmaceutical composition is formulated for parenteral administration. Optionally, the pharmaceutical composition is formulated for intravenous injection.

In some embodiments, the pharmaceutical composition is formulated for inhalation.

In another embodiment, the pharmaceutical composition is formulated for direct injection into the lung tissue.

In certain embodiments, intravenous administration of a nucleic acid-lipid particle or pharmaceutical composition of the disclosure to the subject results in expression of the nucleic acid cargo in cells of the lung tissue of the subject at a level that is at least two-fold higher than expression of the nucleic acid cargo in cells of liver, heart, spleen, ovary, pancreas and kidney of the subject. Optionally, expression of the nucleic acid cargo in cells of the lung tissue of the subject is at least three-fold higher than expression of the nucleic acid cargo in cells of liver, heart, spleen, ovary, pancreas and kidney of the subject. Optionally, expression of the nucleic acid cargo in cells of the lung tissue of the subject is at least four-fold higher than expression of the nucleic acid cargo in cells of liver, heart, spleen, ovary, pancreas and kidney of the subject. Optionally, expression of the nucleic acid cargo in cells of the lung tissue of the subject is at least five-fold higher, at least six-fold higher, at least seven-fold higher, at least eight-fold higher, at least nine-fold higher, at least ten-fold higher, at least eleven-fold higher, at least twelve-fold higher, at least thirteen-fold higher, at least fourteen-fold higher, at least fifteen-fold higher, or at least twenty-fold higher than expression of the nucleic acid cargo in cells of liver, heart, spleen, ovary, pancreas and kidney of the subject.

In some embodiments, intravenous administration of a nucleic acid-lipid particle or pharmaceutical composition of the disclosure to the subject results in localization of the nucleic acid-lipid particle to the lung tissue of the subject at an at least two-fold higher concentration than the concentration of the nucleic acid-lipid particle in one or more of heart, spleen, ovaries and pancreas of the subject. Optionally, at least three-fold, at least four-fold, at least five-fold, or at least six-fold higher concentration of the nucleic acid-lipid particle is located in lung as compared to one or more of heart, spleen, ovaries and pancreas of the subject.

In embodiments, the nucleic acid-lipid particle or pharmaceutical composition is administered to treat a lung disease or disorder. Optionally, the disease or disorder is one or more of: lung cancer, pneumonia, pulmonary fibrosis, COPD, asthma, bronchiectasis, sarcoidosis, pulmonary hypertension, emphysema, alpha-1 antitrypsin deficiency, aspergillosis, bronchiolitis, bronchitis, pneumoconiosis, a coronavirus, Middle Eastern Respiratory Syndrome, Severe Acute Respiratory Syndrome, cystic fibrosis, Legionnaire's disease, influenza, pertussis, pulmonary embolism and tuberculosis.

An additional aspect of the instant disclosure provides a polyethylene glycol (PEG)-free lipid-nucleic acid particle for delivering a nucleic acid cargo to a tissue of a subject, the PEG-free lipid-nucleic acid particle including 1,2-dioleoyl-3-trimethylammonium-propane (DOTAP) at from 20 mol % to 80 mol % of the total lipid present in the PEG-free lipid-nucleic acid particle.

In some embodiments, the PEG-free lipid-nucleic acid particle includes one or more non-cationic lipids at from 20 mol % to 80 mol % of the total lipid present in the PEG-free lipid-nucleic acid particle. Optionally, the non-cationic lipid component of the particle includes cholesterol or a derivative thereof.

In certain embodiments, cholesterol or a derivative thereof is included in the particle at one of the following concentration ranges: about 10 mol % to about 20 mol % of the total lipid present in the particle, about 35 mol % to about 45 mol % of the total lipid present in the particle, and about 60 mol % to about 70 mol % of the total lipid present in the particle.

In one embodiment, the PEG-free lipid-nucleic acid particle includes one or more non-cationic lipid other than cholesterol or a derivative thereof. Optionally, the one or more non-cationic lipid other than cholesterol or a derivative thereof is included at from about 5 mol % to about 20 mol % of the total lipid present in the PEG-free lipid-nucleic acid particle. Optionally, the one or more non-cationic lipid other than cholesterol or a derivative thereof is included at about 10 mol % of the total lipid present in the PEG-free lipid-nucleic acid particle. In a related embodiment, the one or more non-cationic lipid other than cholesterol or a derivative thereof is one or more of the following: 1,2-dioleoyl-sn-glycero-3-phosphocholine (DOPC), 1,2-Distearoyl-sn-glycero-3-phosphocholine (DSPC), 1,2-Dioleoyl-sn-glycero-3-phosphoethanolamine (DOPE) and β-sitosterol. Optionally, the one or more non-cationic lipid other than cholesterol or a derivative thereof is dioleoylphosphatidylcholine (DOPC).

In embodiments, the tissue of the subject is one or more of: lung, joint, epidermis, dermis, endothelium, and blood tissues.

In certain embodiments, a particle of the disclosure is administered parenterally. Optionally, the particle is administered via one or more of the following routes: inhalation, topical application and injection. Optionally, the injection is one or more of the following types: intravenous injection, intratracheal injection or instillation, intra-articular injection, subcutaneous injection, intradermal injection and intramuscular injection.

In some embodiments, the particle (particularly the PEG-free nucleic acid-lipid particle, in view of the tendency of such PEG-free particles to prevent or diminish liver-mediated accelerated blood clearance (ABC) that normally occurs for lipid nanoparticles (LNPs)) is a component of a multi-dose therapy.

Another aspect of the instant disclosure provides a pharmaceutical composition including a PEG-free nucleic acid-lipid particle of the disclosure and a pharmaceutically acceptable carrier.

In embodiments, the pharmaceutical composition is formulated for direct injection into the tissue of the subject.

In some embodiments, the pharmaceutical composition is administered to one or more of the following tissues: lung, joint, epidermis, dermis, endothelium and blood tissue.

In certain embodiments, the pharmaceutical composition is administered to the subject to treat or prevent one or more of the following: a lung disease or disorder, a joint disease or disorder, an inflammatory disease or disorder, and an epidermal disease or disorder.

Optionally, the lung disease or disorder is one or more of: lung cancer, pneumonia, pulmonary fibrosis, chronic obstructive pulmonary disease (COPD), asthma, bronchiectasis, sarcoidosis, pulmonary hypertension, emphysema, alpha-1 antitrypsin deficiency, aspergillosis, bronchiolitis, bronchitis, pneumoconiosis, a coronavirus (e.g., SARS-CoV-2), Middle Eastern Respiratory Syndrome, Severe Acute Respiratory Syndrome, cystic fibrosis, Legionnaire's disease, influenza, pertussis, pulmonary embolism and tuberculosis.

Optionally, the joint disease or disorder is one or more of: rheumatoid arthritis, psoriatic arthritis, gout, tendinitis, bursitis, Carpal Tunnel Syndrome and osteoarthritis.

Optionally, the inflammatory disease or disorder is one or more of: inflammatory bowel disease, peritonitis, osteomyelitis, cachexia, pancreatitis, trauma induced shock, bronchial asthma, allergic rhinitis, cystic fibrosis, acute bronchitis, acute intense bronchitis, osteoarthritis, rheumatoid arthritis, infectious arthritis, post-infectious arthritis, gonocoele arthritis, tuberculous arthritis, arthritis, osteoarthritis, gout, spondyloarthropathies, ankylosing spondylitis, arthritis associated with vasculitis syndrome, nodular polyarteritis nervosa, irritable vasculitis, rugenic granulomatosis, rheumatoid polyposis myalgia, arthritis cell arteritis, calcium polycystic arthropathy, caustic gout, non-arthritic rheumatism, bursitis, hay fever, suppurative inflammation (e.g., tennis elbow), neuropathic joint disease, hemarthrosic, Henoch-Schlein purpura, hypertrophic osteoarthritis, multisized hemorrhoids, scoliosis, hemochromatosis, hyperlipoproteinemia, hypogammaglobulinemia, COPD, acute respiratory distress syndrome, acute lung injury, broncho-pulmonary dysplasia and systemic lupus erythematosus (SLE).

Optionally, the epidermal disease or disorder is one or more of: psoriasis, atopic dermatitis, scleroderma, eczema, rosacea, seborrheic dermatitis, melanoma, solar keratosis, ichthyosis, Grover's disease, common warts, keratoacanthoma and seborrhoeic keratosis.

An additional aspect of the instant disclosure provides a nucleic acid-lipid particle having 1,2-dioleoyl-3-trimethylammonium-propane (DOTAP) at about 45 mol % of the total lipid present in the nucleic acid-lipid particle and having a N/P ratio of about 3.

Another aspect of the instant disclosure provides a nucleic acid-lipid particle having 1,2-dioleoyl-3-trimethylammonium-propane (DOTAP) at about 45 mol % of the total lipid present in the nucleic acid-lipid particle and having a N/P ratio of about 6.

In certain embodiments, the nucleic acid-lipid particle does not include a PEG-lipid conjugate. Optionally, the nucleic acid-lipid particle does not include PEG.

In some embodiments, the nucleic acid-lipid particle includes a conjugated lipid that inhibits aggregation of particles, included at about 1.0% of the total lipid present. Optionally, the conjugated lipid is or includes a polyethyleneglycol (PEG)-lipid conjugate. Optionally, the PEG of the PEG-lipid conjugate has an average molecular weight of from about 550 daltons to about 3000 daltons. Optionally, the PEG-lipid conjugate is a PEG2000-lipid conjugate. Optionally, the PEG2000-lipid conjugate includes one or more of 1,2-dimyristoyl-rac-glycero-3-methoxypolyethylene glycol-2000 (DMG-PEG2k) and 1,2-distearoyl-rac-glycero-3-methoxypolyethylene glycol-2000 (DSG-PEG2k). Optionally, the PEG2000-lipid conjugate is 1,2-Dimyristoyl-rac-glycero-3-methoxypolyethylene glycol-2000 (DMG-PEG2k). Optionally, the nucleic acid-lipid particle includes a PEG-lipid conjugate at a concentration of about 1.0 mol % of the total lipid present in the nucleic acid-lipid particle.

In certain embodiments, the nucleic acid-lipid particle includes a conjugated lipid that inhibits aggregation of particles at about 2.0% of the total lipid present. Optionally, the conjugated lipid includes a polyethyleneglycol (PEG)-lipid conjugate. Optionally, the PEG of the PEG-lipid conjugate has an average molecular weight of from about 550 daltons to about 3000 daltons. Optionally, the PEG-lipid conjugate is a PEG2000-lipid conjugate. Optionally, the PEG2000-lipid conjugate includes one or more of 1,2-dimyristoyl-rac-glycero-3-methoxypolyethylene glycol-2000 (DMG-PEG2k) and 1,2-distearoyl-rac-glycero-3-methoxypolyethylene glycol-2000 (DSG-PEG2k). Optionally, the PEG2000-lipid conjugate is 1,2-Dimyristoyl-rac-glycero-3-methoxypolyethylene glycol-2000 (DMG-PEG2k). Optionally, the nucleic acid-lipid particle includes a PEG-lipid conjugate at a concentration of about 2.0 mol % of the total lipid present in the nucleic acid-lipid particle.

An additional aspect of the instant disclosure provides a nucleic acid-lipid particle having 1,2-dioleoyl-3-trimethylammonium-propane (DOTAP) at about 50 mol % of the total lipid present in the nucleic acid-lipid particle, one or more non-cationic lipid other than cholesterol or a derivative thereof at about 10 mol % of the total lipid present in the nucleic acid-lipid particle and cholesterol or a derivative thereof at about 38 mol % to about 40 mol % of the total lipid present in the nucleic acid-lipid particle.

In certain embodiments, the nucleic acid-lipid particle does not include a PEG-lipid conjugate. Optionally, the nucleic acid-lipid particle includes cholesterol or a derivative thereof at about 39.75 mol % of the total lipid present in the nucleic acid-lipid particle. Optionally, the N/P ratio of the nucleic acid-lipid particle is about 2.

In some embodiments, the nucleic acid-lipid particle includes cholesterol or a derivative thereof at about 39.75 mol % of the total lipid present in the nucleic acid-lipid particle. Optionally, the N/P ratio of the nucleic acid-lipid particle is about 3.

In certain embodiments, the nucleic acid-lipid particle includes a conjugated lipid that inhibits aggregation of particles at about 0.5% of the total lipid present. Optionally, the conjugated lipid includes a polyethyleneglycol (PEG)-lipid conjugate. Optionally, the nucleic acid-lipid particle includes a PEG-lipid conjugate at about 0.5% of the total lipid present in the nucleic acid-lipid particle. Optionally, the nucleic acid-lipid particle includes cholesterol or a derivative thereof at about 39.25 mol % of the total lipid present in the nucleic acid-lipid particle. Optionally, the N/P ratio of the nucleic acid-lipid particle is about 3.

In some embodiments, the nucleic acid-lipid particle includes a conjugated lipid that inhibits aggregation of particles at about 1.0% of the total lipid present. Optionally, the conjugated lipid includes a polyethyleneglycol (PEG)-lipid conjugate. Optionally, the nucleic acid-lipid particle includes a PEG-lipid conjugate at about 1.0% of the total lipid present in the nucleic acid-lipid particle. Optionally, the nucleic acid-lipid particle includes cholesterol or a derivative thereof at about 38.75 mol % of the total lipid present in the nucleic acid-lipid particle. Optionally, the N/P ratio of the nucleic acid-lipid particle is about 3. In an alternative related embodiment, the nucleic acid-lipid particle includes cholesterol or a derivative thereof at about 38.75 mol % of the total lipid present in the nucleic acid-lipid particle. Optionally, the N/P ratio of the nucleic acid-lipid particle is about 2.

In embodiments, the nucleic acid-lipid particle includes a conjugated lipid that inhibits aggregation of particles at about 1.5% of the total lipid present. Optionally, the nucleic acid-lipid particle includes a PEG-lipid conjugate at about 1.5% of the total lipid present in the nucleic acid-lipid particle. Optionally, the nucleic acid-lipid particle includes cholesterol or a derivative thereof at about 38.25 mol % of the total lipid present in the nucleic acid-lipid particle. Optionally, the N/P ratio of the nucleic acid-lipid particle is about 4.

Another aspect of the instant disclosure provides a nucleic acid-lipid particle having 1,2-dioleoyl-3-trimethylammonium-propane (DOTAP) at about 25 mol % of the total lipid present in the nucleic acid-lipid particle, one or more non-cationic lipid other than cholesterol or a derivative thereof at about 10 mol % of the total lipid present in the nucleic acid-lipid particle and cholesterol or a derivative thereof at about 63 mol % to about 65 mol % of the total lipid present in the nucleic acid-lipid particle.

In certain embodiments, the nucleic acid-lipid particle does not include a PEG-lipid conjugate and the nucleic acid-lipid particle includes cholesterol or a derivative thereof at about 64.75 mol % of the total lipid present in the nucleic acid-lipid particle. Optionally, the N/P ratio of the nucleic acid-lipid particle is about 3.

In some embodiments, the nucleic acid-lipid particle includes a conjugated lipid that inhibits aggregation of particles at about 0.5% of the total lipid present. Optionally, the conjugated lipid includes a polyethyleneglycol (PEG)-lipid conjugate. Optionally, the nucleic acid-lipid particle includes a PEG-lipid conjugate at about 0.5% of the total lipid present in the nucleic acid-lipid particle. Optionally, the nucleic acid-lipid particle includes cholesterol or a derivative thereof at about 64.25 mol % of the total lipid present in the nucleic acid-lipid particle. Optionally, the N/P ratio of the nucleic acid-lipid particle is about 4.

In embodiments, the nucleic acid-lipid particle includes a conjugated lipid that inhibits aggregation of particles at about 1.0% of the total lipid present. Optionally, the conjugated lipid includes a polyethyleneglycol (PEG)-lipid conjugate. Optionally, the nucleic acid-lipid particle includes a PEG-lipid conjugate at about 1.0% of the total lipid present in the nucleic acid-lipid particle. Optionally, the nucleic acid-lipid particle includes cholesterol or a derivative thereof at about 63.75 mol % of the total lipid present in the nucleic acid-lipid particle. Optionally, the N/P ratio of the nucleic acid-lipid particle is about 4.

In some embodiments, the nucleic acid-lipid particle includes a conjugated lipid that inhibits aggregation of particles at about 1.5% of the total lipid present. Optionally, the nucleic acid-lipid particle includes a PEG-lipid conjugate at about 1.5% of the total lipid present in the nucleic acid-lipid particle. Optionally, the nucleic acid-lipid particle includes cholesterol or a derivative thereof at about 63.25 mol % of the total lipid present in the nucleic acid-lipid particle. Optionally, the N/P ratio of the nucleic acid-lipid particle is about 2.

An additional aspect of the instant disclosure provides a nucleic acid-lipid particle having 1,2-dioleoyl-3-trimethylammonium-propane (DOTAP) at about 75 mol % of the total lipid present in the nucleic acid-lipid particle, one or more non-cationic lipid other than cholesterol or a derivative thereof at about 10 mol % of the total lipid present in the nucleic acid-lipid particle and cholesterol or a derivative thereof at about 13 mol % to about 15 mol % of the total lipid present in the nucleic acid-lipid particle.

In certain embodiments, the nucleic acid-lipid particle does not include a PEG-lipid conjugate and the nucleic acid-lipid particle includes cholesterol or a derivative thereof at about 14.75 mol % of the total lipid present in the nucleic acid-lipid particle. Optionally, the N/P ratio of the nucleic acid-lipid particle is about 4.

In some embodiments, the nucleic acid-lipid particle includes a conjugated lipid that inhibits aggregation of particles at about 0.5% of the total lipid present. Optionally, the conjugated lipid includes a polyethyleneglycol (PEG)-lipid conjugate. Optionally, the nucleic acid-lipid particle includes a PEG-lipid conjugate at about 0.5% of the total lipid present in the nucleic acid-lipid particle. Optionally, the nucleic acid-lipid particle includes cholesterol or a derivative thereof at about 14.25 mol % of the total lipid present in the nucleic acid-lipid particle. Optionally, the N/P ratio of the nucleic acid-lipid particle is about 2.

In embodiments, the nucleic acid-lipid particle includes a conjugated lipid that inhibits aggregation of particles at about 1.0% of the total lipid present. Optionally, the conjugated lipid includes a polyethyleneglycol (PEG)-lipid conjugate. Optionally, the nucleic acid-lipid particle includes a PEG-lipid conjugate at about 1.0% of the total lipid present in the nucleic acid-lipid particle. Optionally, the nucleic acid-lipid particle includes cholesterol or a derivative thereof at about 13.75 mol % of the total lipid present in the nucleic acid-lipid particle. Optionally, the N/P ratio of the nucleic acid-lipid particle is about 2.

In some embodiments, the nucleic acid-lipid particle includes a conjugated lipid that inhibits aggregation of particles at about 1.5% of the total lipid present. Optionally, the nucleic acid-lipid particle includes a PEG-lipid conjugate at about 1.5% of the total lipid present in the nucleic acid-lipid particle. Optionally, the nucleic acid-lipid particle includes cholesterol or a derivative thereof at about 13.25 mol % of the total lipid present in the nucleic acid-lipid particle. Optionally, the N/P ratio of the nucleic acid-lipid particle is about 3.

In certain embodiments, the one or more non-cationic lipid other than cholesterol or a derivative thereof includes one or more of the following: 1,2-dioleoyl-sn-glycero-3-phosphocholine (DOPC), 1,2-Distearoyl-sn-glycero-3-phosphocholine (DSPC), 1,2-Dioleoyl-sn-glycero-3-phosphoethanolamine (DOPE) and β-sitosterol. Optionally, the one or more non-cationic lipid other than cholesterol or a derivative thereof is dioleoylphosphatidylcholine (DOPC).

Another aspect of the instant disclosure provides an injectate that includes the nucleic acid-lipid particle, pharmaceutical composition or PEG-free lipid-nucleic acid particle of the instant disclosure.

An additional aspect of the instant disclosure provides a method for delivering a nucleic acid cargo to a lung tissue of a subject that includes administering the nucleic acid-lipid particle, pharmaceutical composition, PEG-free lipid-nucleic acid particle or injectate of the instant disclosure to the subject.

A further aspect of the instant disclosure provides a method for treating or preventing a disease or disorder in a subject, the method including administering the nucleic acid-lipid particle, pharmaceutical composition, PEG-free lipid-nucleic acid particle or injectate of the instant disclosure the subject.

In embodiments, the nucleic acid-lipid particle, pharmaceutical composition, PEG-free lipid-nucleic acid particle or injectate is administered intravenously and expression of the nucleic acid cargo in cells of the lung tissue of the subject occurs at a level that is at least two-fold higher than expression of the nucleic acid cargo in cells of liver, heart, spleen, ovary, pancreas and/or kidney of the subject. Optionally, expression of the nucleic acid cargo in cells of the lung tissue of the subject is at least three-fold higher, optionally at least four-fold higher, optionally at least five-fold higher, optionally at least six-fold higher, optionally at least seven-fold higher, optionally at least eight-fold higher, optionally at least nine-fold higher, optionally at least ten-fold higher, optionally at least eleven-fold higher, optionally at least twelve-fold higher, optionally at least thirteen-fold higher, optionally at least fourteen-fold higher, optionally at least fifteen-fold higher, optionally at least twenty-fold higher, than expression of the nucleic acid cargo in cells of liver, heart, spleen, ovary, pancreas and kidney of the subject.

In some embodiments, the nucleic acid-lipid particle, pharmaceutical composition, PEG-free lipid-nucleic acid particle or injectate is administered intravenously and the nucleic acid-lipid particle or PEG-free lipid-nucleic acid particle localizes to the lung tissue of the subject at an at least two-fold higher concentration than the concentration of the nucleic acid-lipid particle or PEG-free lipid-nucleic acid particle in one or more of the following other tissues of the subject: heart, spleen, ovaries and pancreas. Optionally, at least three-fold, optionally at least four-fold, optionally at least five-fold, optionally at least six-fold higher concentration of the nucleic acid-lipid particle or PEG-free lipid-nucleic acid particle is present in lung as compared to the concentration of the nucleic acid-lipid particle or PEG-free lipid-nucleic acid particle in one or more of the following other tissues of the subject: heart, spleen, ovaries and pancreas.

### Definitions

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value.

In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Unless otherwise clear from context, all numerical values provided herein are modified by the term “about.”

The term “lipid” refers to a group of organic compounds that include, but are not limited to, esters of fatty acids and are characterized by being insoluble in water, but soluble in many organic solvents. They are usually divided into at least three classes: (1) “simple lipids” which include fats and oils as well as waxes; (2) “compound lipids” which include phospholipids and glycolipids; (3) “derived lipids” such as steroids.

As used herein, the term “cationic lipid” refers to any of a number of lipid species that carry a net positive charge at a selected pH, such as physiological pH. Cationic lipids include those lipids and salts thereof having one, two, three, or more fatty acid or fatty alkyl chains and a pH-titratable amino head group (e.g., an alkylamino or dialkylamino head group). The cationic lipid is typically protonated (i.e., positively charged) at a pH below the pKa of the cationic lipid and is substantially neutral at a pH above the pKa. The cationic lipids of the description herein may also be termed titratable cationic lipids. In some embodiments, the cationic lipids comprise: a protonatable tertiary amine (e.g., pH-titratable) head group; C18 alkyl chains, wherein each alkyl chain independently has 0 to 3 (e.g., 0, 1, 2, or 3) double bonds; and ether, ester, or ketal linkages between the head group and alkyl chains. Such cationic lipids include, but are not limited to, DOTAP, 1,2-distearyloxy-N,N-dimethyl-3-aminopropane (DSDMA), 1,2-dioleyloxy-N,N-dimethyl-3-aminopropane (DODMA), 1,2-dilinoleyloxy-N,N-dimethyl-3-aminopropane (DLinDMA), 1,2-dilinolenyloxy-N,N-dimethyl-3-aminopropane (DLenDMA), 1,2-di-γ-linolenyloxy-N,N-dimethylaminopropane (γ-DLenDMA, 1,2-dilinoleyloxy-keto-N,N-dimethyl-3-aminopropane (DLinK-DMA), 1,2-dilinoleyl-4-(2-dimethylaminoethyl)-[1,3]-dioxolane (DLinKC2-DMA) (also known as DLin-C2K-DMA, XTC2, and C2K), 2,2-dilinoleyl-4-(3-dimethylaminopropyl)[1,3]-dioxolane (DLin-K-C3-DMA), 2,2-dilinoleyl-4-(4-dimethylaminobutyl)[1,3]-dioxolane (DLin-K-C4-DMA), 1,2-dilinolenyloxy-4-(2-dimethylaminoethyl)-[1,3]-dioxolane (γ-DLen-C2K-DMA), 1,2-di-γ-linolenyloxy-4-(2-dimethylaminoethyl)-[1,3]-dioxolane (γ-DLen-C2K-DMA), dilinoleylmethyl-3-dimethylaminopropionate (DLin-M-C2-DMA) (also known as MC2), (6Z,9Z,28Z,31Z)-heptatriaconta-6,9,28,31-tetraen-19-yl 4-(dimethylamino) butanoate (DLin-M-C3-DMA) (also known as MC3) and 3-(dilinoleylmethoxy)-N,N-dimethylpropan-1-amine (DLin-MP-DMA) (also known as 1-11). As used herein, “DOTAP,” refers to 1,2-dioleoyl-3-trimethylammonium-propane, or 18:1 TAP, a di-chain, or gemini, cationic lipid.

DOTAP is a cationically charged lipid independent of pH, due to its quaternary structure. It is sold commercially for the liposomal-transfection of DNA, RNA and other negatively charged molecules. In some aspects of the instant disclosure, DOTAP lipids, or variations thereof, are used in lipid nanoparticles to deliver nucleic acids specifically to the lung. In other aspects, DOTAP lipids, or variations thereof, are used in lipid nanoparticles to deliver nucleic acids to joints, inflammation sites, the epidermis, and the dermis. The structure of DOTAP (C₄₂H₈₀NO₄⁺) is shown below:

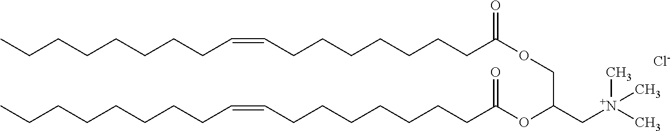 [SMILES: CCCCCCCC/C=C\CCCCCCCC(=O)OCC(C[N+](C)(C)C)OC(=O)CCCCCCC/C=C\CCCCCCCC ; [Cl-]]

As used herein, the term “non-cationic lipid” refers to any neutral lipid, as well as any anionic lipids. A “neutral lipid” refers to any of a number of lipid species that exist either in an uncharged or neutral zwitterionic form at a selected pH. At physiological pH, such lipids include, for example, diacylphosphatidylcholine, diacylphosphatidylethanolamine, ceramide, sphingomyelin, cephalin, cholesterol, cerebrosides and diacylglycerols. An “anionic lipid” refers to any lipid that is negatively charged at physiological pH. These lipids include, but are not limited to, phosphatidylglycerols, cardiolipins, diacylphosphatidylserines, diacylphosphatidic acids, N-dodecanoyl phosphatidylethanolamines, N-succinyl phosphatidylethanolamines, N-glutarylphosphatidylethanolamines, lysylphosphatidylglycerols, palmitoyloleyolphosphatidylglycerol (POPG), and other anionic modifying groups joined to neutral lipids. In some embodiments, the non-cationic lipid used in the instant disclosure is 1,2-dioleoyl-sn-glycero-3-phosphocholine (DOPC), 1,2-Distearoyl-sn-glycero-3-phosphocholine (DSPC), and/or 1,2-Dioleoyl-sn-glycero-3-phosphoethanolamine (DOPE). In embodiments, the non-cationic lipid is cholesterol (CHE) and/or β-sitosterol.

The term “lipid nanoparticle” as used herein refers to different types of compositions of nano-scale particles, wherein the particles comprising lipids function as carriers across cell membranes and biological barriers and deliver compounds to targeted cells and tissues of humans and other organisms. As used herein, “lipid nanoparticles” of the instant disclosure may further comprise additional lipids and other components. Other lipids may be included for a variety of purposes, such as to prevent lipid oxidation or to attach ligands onto the lipid nanoparticle surface. Any of a number of lipids may be present in lipid nanoparticles of the present disclosure, including amphipathic, neutral, cationic, and anionic lipids. Such lipids can be used alone or in combination, and can also include bilayer stabilizing components such as polyamide oligomers (see, e.g., U.S. Pat. No. 6,320,017), peptides, proteins, detergents, lipid-derivatives, such as PEG coupled to phosphatidylethanolamine and PEG conjugated to ceramides (see, e.g., U.S. Pat. No. 5,885,613).

As used herein, a “PEG” conjugated lipid that inhibits aggregation of particles refers to one or more of a polyethyleneglycol (PEG)-lipid conjugate, a polyamide (ATTA)-lipid conjugate, and a mixture thereof. In one aspect, the PEG-lipid conjugate is one or more of a PEG-dialkyloxypropyl (DAA), a PEG-diacylglycerol (DAG), a PEG-phospholipid, a PEG-ceramide, and a mixture thereof. In one aspect, the PEG-DAG conjugate is one or more of a PEG-dilauroylglycerol (C₁₂), a PEG-dimyristoylglycerol (C₁₄), a PEG-dipalmitoylglycerol (C₁₆), and a PEG-distearoylglycerol (C₁₈). In one aspect, the PEG-DAA conjugate is one or more of a PEG-dilauryloxypropyl (C₁₂), a PEG-dimyristyloxypropyl (C₁₄), a PEG-dipalmityloxypropyl (C₁₆), and a PEG-di stearyloxypropyl (C₁₈). In some embodiments, PEG is 2-dimyristoyl-rac-glycero-3-methoxypolyethylene glycol-2000 (PEG-DMG) and/or 1,2-distearoyl-rac-glycero-3-methoxypolyethylene glycol-2000 (PEG-DSG).

The term “N/P ratio” as used herein refers to the (N)itrogen-to-(P)hosphate ratio between the cationic amino lipid and negatively charged phosphate groups of the nucleic acid.

The “polydispersity index” or “PDI” as used herein is a measure of the heterogeneity of a sample based on size. Polydispersity can occur due to size distribution in a sample or agglomeration or aggregation of the sample during isolation or analysis.

The “zeta potential” or “surface charge” as used herein refers to the degree of electrostatic repulsion between adjacent, similarly charged particles in a dispersion. For molecules and particles that are small enough, a high zeta potential will confer stability, i.e., the solution or dispersion will resist aggregation.

As used herein, the term nucleic acid “cargo” is the intended therapeutic nucleic acid for delivery to the cell or tissue.

As used herein, the term “nucleic acid-lipid nanoparticle” refers to lipid nanoparticles as described above that associate with or encapsulate one or more nucleic acids to deliver one or more therapeutic nucleic acid cargoes to a tissue.

As used herein, “encapsulated” can refer to a nucleic acid-lipid nanoparticle formulation that provides a nucleic acid with full encapsulation, partial encapsulation, association by ionic or van der Waals forces, or all of the aforementioned. In a preferred embodiment, the nucleic acid is fully encapsulated in the nucleic acid-lipid nanoparticle.

As used herein, “nucleic acid” refers to a synthetic or naturally occurring RNA or DNA, or derivatives thereof. In one embodiment, a cargo and/or agent of the instant disclosure is a nucleic acid, such as a double-stranded RNA (dsRNA). In one embodiment, the nucleic acid or nucleic acid cargo is a single-stranded DNA or RNA, or double-stranded DNA or RNA, or DNA-RNA hybrid. For example, a double-stranded DNA can be a structural gene, a gene including control and termination regions, or a self-replicating system such as a viral or plasmid DNA. A double-stranded RNA can be, e.g., a dsRNA or another RNA interference reagent. A single-stranded nucleic acid can be, e.g., an mRNA, an antisense oligonucleotide, ribozyme, a microRNA, or triplex-forming oligonucleotide. In certain embodiments, the nucleic acid or nucleic acid cargo may comprise a modified RNA, wherein the modified RNA is one or more of a modified mRNA, a modified antisense oligonucleotide and a modified siRNA. In some embodiments, a nucleic acid cargo of the instant disclosure includes or is a modified mRNA that encodes a nucleic acid modulating controller.

As used herein, the term “modified nucleic acid” refers to any non-natural nucleic acid, including but not limited to those selected from the group comprising 2′-O-methyl modified nucleotides, a nucleotide comprising a 5′-phosphorothioate group, a terminal nucleotide linked to a cholesteryl derivative, a 2′-deoxy-2′-fluoro modified nucleotide, a 5′-methoxy-modified nucleotide (e.g., 5′-methoxyuridine), a 2′-deoxy-modified nucleotide, a locked nucleotide, an abasic nucleotide, a 2′-amino-modified nucleotide, a 2′-alkyl-modified nucleotide, a morpholino nucleotide, a phosphoramidate, a non-natural base comprising nucleotide; internucleoside linkages or backbones including phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′.

As used herein, the term “nucleic acid modulating controller” refers to a mRNA that encodes for protein controller components, though reference to “nucleic acid modulating controller” can also refer to the mRNA-expressed protein controller components themselves. In certain embodiments, the mRNA-encoded protein controller components include Zinc-Finger proteins (ZFPs) or other forms of DNA or RNA binding domains (DBDs or RBDs) that are associated with (and optionally tethered to) one or more epigenetic regulators or nucleases (the epigenetic regulators or nucleases are generally referred to as effectors, effector domains, or effector moieties). Without wishing to be bound by theory, an advantage of a nucleic acid modulating controller as described herein is that it provides durable gene programming only at the confluence of (1) where the nucleic acid modulating controller-encoding mRNA is expressed, (2) where nucleic acid binding of the ZFP or other nucleic acid binding domain occurs and (3) where the associated effector domain is able to exert activity (i.e. where the effector domain is capable of changing the epigenomic state (e.g., in the instance of an epigenomic controller)).

As used herein, the term “effector moiety” or “effector domain” refers to a domain that is capable of altering the expression of a target gene when localized to an appropriate site in a cell, e.g., in the nucleus of a cell. In some embodiments, an effector moiety recruits components of the transcription machinery. In some embodiments, an effector moiety inhibits recruitment of components of transcription factors or expression repressing factors. In some embodiments, an effector moiety comprises an epigenetic modifying moiety (e.g., epigenetically modifies a target DNA sequence). Specific examples of effector moieties include, without limitation, effectors capable of binding Krueppel-associated box (KRAB) domains (KRAB is a domain of around 75 amino acids that is found in the N-terminal part of about one third of eukaryotic Krueppel-type C2H2 zinc finger proteins (ZFPs)) and the engineered prokaryotic DNA methyltransferase MQ1, among others.

As used herein, “epigenetic modifying moiety” refers to a domain that alters: i) the structure, e.g., two dimensional structure, of chromatin; and/or ii) an epigenetic marker (e.g., one or more of DNA methylation, histone methylation, histone acetylation, histone sumoylation, histone phosphorylation, and RNA-associated silencing), when the epigenetic modifying moiety is appropriately localized to a nucleic acid (e.g., by a targeting moiety). In some embodiments, an epigenetic modifying moiety comprises an enzyme, or a functional fragment or variant thereof, that affects (e.g., increases or decreases the level of) one or more epigenetic markers. In some embodiments, an epigenetic modifying moiety comprises a DNA methyltransferase, a histone methyltransferase, CREB-binding protein (CBP), or a functional fragment of any thereof.

As used herein, the term “expression control sequence” refers to a nucleic acid sequence that increases or decreases transcription of a gene, and includes (but is not limited to) a promoter and an enhancer. An “enhancing sequence” refers to a subtype of expression control sequence and increases the likelihood of gene transcription. A “silencing or repressor sequence” refers to a subtype of expression control sequence and decreases the likelihood of gene transcription.

As used herein, the term “expression repressor” refers to an agent or entity with one or more functionalities that decreases expression of a target gene in a cell and that specifically binds to a DNA sequence (e.g., a DNA sequence associated with a target gene or a transcription control element operably linked to a target gene). In certain embodiments, an expression repressor comprises at least one targeting moiety and optionally one effector moiety.

As used herein, the term “targeting moiety” means an agent or entity that specifically targets, e.g., binds, a genomic sequence element (e.g., an expression control sequence or anchor sequence; promoter, enhancer or CTCF site). In some embodiments, the genomic sequence element is proximal to and/or operably linked to a target gene (e.g., MYC).

As used herein, “lung tissue” may refer to any cell within the organ of the lung including but not limited to the group comprising the epithelium, endothelium, interstitial connective tissue, blood vessel, hematopoietic tissue, lymphoid tissue, and pleura. In preferred embodiments, the nucleic acid-lipid nanoparticle targets lung tissue. In some other embodiments, the nucleic acid-lipid nanoparticle may target other cells or tissues including but not limited to brain, nerve, skin, eye, pharynx, larynx, heart, vascular, hematopoietic (e.g., white blood cell or red blood cell), breast, liver, pancreas, spleen, esophagus, gall bladder, stomach, intestine, colon, kidney, urinary bladder, ovary, uterus, cervix, prostate, muscle, bone, thyroid, parathyroid, adrenal, and pituitary cells or tissues.

As used herein, “localization” refers to the position of a lipid, peptide, or other component of a lipid particle of the instant disclosure, within an organism and/or tissue. In some embodiments, localization can be detectible in individual cells. In some embodiments a label can be used for detecting localization, e.g., a fluorescent label, optionally a fluorescently labeled lipid, optionally Cy7. In some embodiments, the label of the lipid nanoparticle may be a quantum dot, or the lipid detectible by stimulated Raman scattering. In other embodiments, the label is any fluorophore known in the art, i.e. with excitation and emission in the ultraviolet, visible, or infrared spectra. In some embodiments the localization is detected or further corroborated by immunohistochemistry or immunofluorescence.

As used herein, the term “activity” refers to any detectable effect that is mediated by a component or composition of the instant disclosure. In embodiments, “activity” as used herein, can refer to a measurable (whether directly or by proxy) effect, e.g., of a cargo of the instant lipid particles of the disclosure. Examples of activity include, without limitation, the intracellular expression and resulting effect(s) of a nucleic acid cargo (e.g., a mRNA, a CRISPR/Cas system, a RNAi agent, a nucleic acid modulating controller, etc.), which can optionally be measured at a cellular, tissue, organ and/or organismal level.

As used herein, “accelerated blood clearance” or “ABC” refers to a well-documented phenomenon caused by immune system activation against PEG molecules on the surface of LNPs. ABC is responsible for the clearance of nanoparticles from systemic circulation upon repeated dosing. In some embodiments, lipid particles of the instant disclosure can avoid or reduce accelerated blood clearance of lipid particles, by employing PEG-free formulations, which can also provide for improved (e.g., less toxic and/or more effective) repeated systemic administration of such lipid particles. As used herein, “multidosing” refers to two or more doses of a lipid nanoparticle formulation given as part of a therapeutic regimen to a subject.

As used herein, the term “lung disease or disorder” may include, without limitation, a disease or disorder selected from the following: lung cancer, pneumonia, pulmonary fibrosis, COPD, asthma, bronchiectasis, sarcoidosis, pulmonary hypertension, emphysema, alpha-1 antitrypsin deficiency, aspergillosis, bronchiolitis, bronchitis, pneumoconiosis, Coronaviruses, Middle Eastern Respiratory Syndrome, Severe Acute Respiratory Syndrome, cystic fibrosis, Legionairre's disease, influenza, pertussis, pulmonary embolism, and tuberculosis.

As used herein, a “joint diseases or disorder,” may include, without limitation, a disease or disorder selected from the following: rheumatoid arthritis, psoriatic arthritis, gout, tendinitis, bursitis, Carpal Tunnel Syndrome, and osteoarthritis.

As used herein, an “inflammatory disease or disorder,” may include, without limitation, a disease or disorder selected from the following: inflammatory bowel disease, peritonitis, osteomyelitis, cachexia, pancreatitis, trauma induced shock, bronchial asthma, allergic rhinitis, cystic fibrosis, acute bronchitis, acute intense bronchitis, osteoarthritis, rheumatoid arthritis, infectious arthritis, post-infectious arthritis, gonocoele arthritis, tuberculous arthritis, arthritis, osteoarthritis, gout, spondyloarthropathies, ankylosing spondylitis, arthritis associated with vasculitis syndrome, nodular polyarteritis nervosa, irritable vasculitis, rugenic granulomatosis, rheumatoid polyposis myalgia, arthritis cell arteritis, calcium polycystic arthropathy, caustic gout, non-arthritic rheumatism, bursitis, hay fever, suppurative inflammation (e.g., tennis elbow), neuropathic joint disease, hemarthrosic, Henoch-Schlein purpura, hypertrophic osteoarthritis, multisized hemorrhoids, scoliosis, hemochromatosis, hyperlipoproteinemia, hypogammaglobulinemia, COPD, acute respiratory distress syndrome, acute lung injury, broncho-pulmonary dysplasia and systemic lupus erythematosus (SLE).

As used herein, an “epidermal disease or disorder,” may include, without limitation, a disease or disorder selected from the following: psoriasis, atopic dermatitis, scleroderma, eczema, rosacea, seborrheic dermatitis, melanoma, solar keratosis, ichthyosis, Grover's disease, common warts, keratoacanthoma, and seborrhoeic keratosis.

As used herein, the term “subject” includes humans and mammals (e.g., mice, rats, pigs, cats, dogs, and horses). In many embodiments, subjects are mammals, particularly primates, especially humans. In some embodiments, subjects are livestock such as cattle, sheep, goats, cows, swine, and the like; poultry such as chickens, ducks, geese, turkeys, and the like; and domesticated animals particularly pets such as dogs and cats. In some embodiments (e.g., particularly in research contexts) subject mammals will be, for example, rodents (e.g., mice, rats, hamsters), rabbits, primates, or swine such as inbred pigs and the like.

As used herein, “administration” to a subject may include parenteral administration, optionally for intravenous injection, inhalation, intravenous, intra-arterial, intratracheal, topical, or involve direct injection into a tissue.

The term “treating” includes the administration of compositions to prevent or delay the onset of the symptoms, complications, or biochemical indicia of a disease (e.g., cancer, including, e.g., tumor formation, growth and/or metastasis), alleviating the symptoms or arresting or inhibiting further development of the disease, condition, or disorder. Treatment may be prophylactic (to prevent or delay the onset of the disease, or to prevent the manifestation of clinical or subclinical symptoms thereof) or therapeutic suppression or alleviation of symptoms after the manifestation of the disease.

As used herein, a “pharmaceutical composition” comprises a pharmacologically effective amount of a lipid particle, optionally a nucleic-acid lipid nanoparticle (NLNP) and a pharmaceutically acceptable carrier. As used herein, “pharmacologically effective amount,” “therapeutically effective amount” or simply “effective amount” refers to that amount of nucleic acid effective to produce the intended pharmacological, therapeutic or preventive result. For example, if a given clinical treatment is considered effective when there is at least a 25% reduction in a measurable parameter associated with a disease or disorder, a therapeutically effective amount of a drug for the treatment of that disease or disorder is the amount necessary to induce at least a 25% reduction in that parameter.

The term “pharmaceutically acceptable carrier” refers to a carrier for administration of a therapeutic agent. Such carriers include, but are not limited to, saline, buffered saline, dextrose, water, glycerol, ethanol, and combinations thereof.

Unless specifically stated or obvious from context, as used herein, the term “or” is understood to be inclusive. Unless specifically stated or obvious from context, as used herein, the terms “a”, “an”, and “the” are understood to be singular or plural.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it is understood that the particular value forms another aspect. It is further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. It is also understood that throughout the application, data are provided in a number of different formats and that this data represent endpoints and starting points and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

The embodiments set forth below and recited in the claims can be understood in view of the above definitions.

Other features and advantages of the disclosure will be apparent from the following description of the preferred embodiments thereof, and from the claims. Unless otherwise defined, all technical and scientific terms used herein have the same meaning as commonly understood by one of ordinary skill in the art to which this disclosure belongs. Although methods and materials similar or equivalent to those described herein can be used in the practice or testing of the present disclosure, suitable methods and materials are described below. All published foreign patents and patent applications cited herein are incorporated herein by reference. All other published references, documents, manuscripts and scientific literature cited herein are incorporated herein by reference. In the case of conflict, the present specification, including definitions, will control. In addition, the materials, methods, and examples are illustrative only and not intended to be limiting.

### BRIEF DESCRIPTION OF THE DRAWINGS

The following detailed description, given by way of example, but not intended to limit the disclosure solely to the specific embodiments described, may best be understood in conjunction with the accompanying drawings, in which:

FIGS. 1A and 1B show that DOTAP lipid nanoparticles (LNPs) delivered reporter mRNA cargoes and exhibited low toxicity in vitro. FIG. 1A shows the observed luciferase enzyme activities of four DOTAP LNP formulations tested in a murine cell line (Hepa 1-6), across indicated cargo mRNA mFluc (luciferase) concentration ranges. Remarkably, an approximately 600-fold increase in luciferase activity was achieved with PEG-free formulations (0% PEG) at concentrations of 0.625 μg/ml, 1.25 μg/ml and 2.5 μg/ml. Dose-dependence of cargo mRNA activity was observed for all LNP formulations examined, with progressively increasing levels of delivery and expression of the mFluc (luciferase) cargo also observed for the PEG-containing LNP formulations tested. FIG. 1B shows the effects of high concentrations of the tested DOTAP-LNPs on Hepa 1-6 cell viability, with robust viability observed for PEG-free DOTAP LNPs, and only slightly diminished viability observed for increasing concentrations of the PEG-containing NP:6PEG:1 LNP formulation that was tested.

FIGS. 2A-2F show that DOTAP lipid nanoparticles (LNPs) robustly localized to and expressed mRNA cargo in the lungs of treated mice, when LNPs formulated with reporter mRNA as cargo were administered intravenously. FIG. 2A shows that two different tested DOTAP LNPs, NP:3PEG:0 and NP:3PEG:1 (also shown in Table 1), exhibited concentrated luciferase activity in mouse lungs, with observed effects persisting for 24 h. FIG. 2B shows the results of luminescence and fluorescence imaging performed ex vivo upon major organs harvested from treated mice. Cy7 signal distribution indicates LNP biodistribution, while the luminescence signal indicates reporter mRNA cargo expression and activity. Notably, lung levels of cargo mRNA expression were particularly robust, even as DOTAP-LNPs distributed well to a number of tissues. FIG. 2C shows quantification of the observed Cy7-DOPE lipid luminescent biodistribution signal in harvested mouse organs. FIG. 2D shows quantification of the luminescence signal from the expression of mFluc mRNA in the mice organs—specifically, the observed percent distribution of the luciferase activity within the organs is shown, with percent values calculated by using the total summed signal coming from all organs, then calculating the percent signal for each individual organ corresponding to the total value. Strong specificity of DOTAP-LNPs to lungs (>90% of the activity was localized in the lungs) was thereby documented. FIG. 2E shows the luciferase activity values in the lungs as Average Radiance, to represent the collected signal from lungs as representative readings of direct activity. Notably, DOTAP-LNPs with 0% PEG exhibited approximately 50-fold higher mRNA expression (luciferase activity) in the lungs than did DOTAP-LNPs with 1% PEG. FIG. 2F shows that DOTAP-LNPs did not cause significant body weight change when administered to mice via intravenous injection. FIG. 2G demonstrates that liver function tests for alkaline phosphatase (ALP), aspartate transaminase (ALT), and aspartate aminotransferase (AST) following DOTAP-LNP administration showed no significant elevation, as compared to PBS control-treated animals.

FIGS. 3A-3G show that lung-selective localization of DOTAP-LNPs and associated mRNA cargo expression were observed for DOTAP-LNPs possessing all tested PEG-lipid chemistries. FIG. 3A shows that, independent of the PEG-lipid type, mRNA cargo-directed luciferase activity occurred preferentially in the lungs of injected subject mice at 24, 48 and 72 hour timepoints. Both PEG-DSG and PEG-DMG formulations showed wide distribution of the LNPs, with kidneys being a major organ of LNP accumulation. These results supported that the kidneys are a primary excretion route for LNPs. At bottom, lungs were removed from the ex vivo assessment of bioluminescence radiance, and the remaining organs were re-imaged to obtain a higher signal-to-background ratio of luciferase radiance, as a high signal from a particular organ can mask lower but still significant signals from other organs. FIG. 3B shows quantification of Cy7 lipid imaging results, which demonstrated that kidneys were the major organ for LNP accumulation (even if no significant mRNA cargo expression was observed in kidneys). FIG. 3C shows quantification of the mRNA reporter mFluc luminescence for both PEG-DSG and PEG-DMG formulations of DOTAP-LNP, which demonstrated that for both formulations, and over all time points, DOTAP-LNP delivery-mediated mRNA cargo expression occurred almost 100% in the lungs. FIG. 3D shows that no significant body weight changes were observed following DOTAP-LNP dosing with either PEG-DSG or PEG-DMG lipids. FIG. 3E shows that alkaline phosphatase (ALP) as a test of liver function remained stable for both PEG-lipid DOTAP LNP formulations after administration (24, 48, and 72 hour time points). FIG. 3F shows that aspartate transaminase transferase (ALT) as a test of liver function also remained stable for both PEG-lipid DOTAP LNP formulations after administration (24, 48, and 72 hour time points). Similarly, FIG. 3G shows that aspartate aminotransferase (AST) as a test of liver function remained stable for both PEG-lipid DOTAP LNP formulations after administration (24, 48, and 72 hour time points).

FIGS. 4A-4C demonstrate that DOTAP-LNPs successfully delivered a Cre mRNA reporter system as a nucleic acid cargo to cells. FIG. 4A shows dose dependent cellular association of tested DOTAP-LNPs with the HEK293-loxP-GFP-RFP cell line. This cell line stably expressed GFP signal, yet upon expression of Cre recombinase in the cells, due to loxP recombination, the cells began to express RFP instead of GFP. FIG. 4B shows an image that demonstrates that, following treatment of the HEK293-loxP-GFP-RFP cell line with DOTAP-LNPs harboring a Cre mRNA reporter system, successfully transfected cells expressed RFP instead of GFP. FIG. 4C shows that mCre activity was also confirmed with flow cytometry, which demonstrated decreased GFP signal in the cells.

FIGS. 5A and 5B show that DOTAP-LNPs delivered and expressed nucleic acid cargoes targeting the lung genome in vivo. FIG. 5A shows ex vivo organ imaging results 48 hours and 72 hours after dosing. Ex vivo imaging at both time points demonstrated that tested DOTAP-LNPs exhibited lung-specific activity (tdTomato), regardless of particle distribution (Cy7). FIG. 5B summarizes the signal quantification from the imaging studies. As shown in the graphs, despite the approximately equal distribution of the LNPs between liver and lungs (Cy7 radiance), mCre activity (tdTomato radiance, reflecting mCre expression) was only observed in the lungs (with the exception of one animal outlier).

FIGS. 6A-6C show that DOTAP-LNPs transduced all cell types in lung tissue, including inflamed lung tissue. FIG. 6A shows the observed tdTomato signal in the heart, lungs, liver, kidneys, pancreas and spleen harvested from an Ail4 mouse (healthy mouse), comparing untreated, MC3 LNP-treated and DOTAP-LNP-treated animals. Lungs from the Ail4 animals treated intravenously with mCre mRNA cargo-loaded DOTAP-LNPs having 1% PEG-DMG exhibited robust, lung-specific tdTomato expression and were then evaluated with immunohistochemistry methods (IHC) to evaluate tdTomato expression levels in different cell populations. FIG. 6B shows a histology staining for tdTomato in the lung of healthy DOTAP-LNP-administered animals. Macrophage, epithelial, and endothelial cells of lung were all visibly transduced with cargo mRNA, demonstrating that DOTAP-LNPs transduced both progenitor and epithelial cells after IV administration. FIG. 6C shows immunohistochemistry stained tissue sections for DOTAP-LNP-administered mice having inflamed lungs (NSG-SGM3 mouse), respectively showing (from left to right) tdTomato immunohistochemistry (DOTAP-LNP-delivered mCre mRNA expression as a marker for delivery), duplex immunohistochemistry of tdTomato with mouse CD45 (epithelial, alveolar cells and CD45⁺ cells (monocytes, neutrophils) indicated by arrows), duplex immunohistochemistry of tdTomato with human CD45 (epithelial, alveolar cells and CD45⁺ cells (monocytes, neutrophils) indicated by arrows), duplex immunohistochemistry of tdTomato with human CD68 (macrophage and alveolar cells indicated by arrows), and duplex immunohistochemistry of tdTomato with neutrophil elastase (macrophage and neutrophils indicated by arrows).

FIGS. 7A-7E show that different DOTAP-LNP formulations exhibited improved nucleic acid cargo delivery to the lungs. FIG. 7A shows that both the 3450 and 4750 formulations of the instant disclosure exhibited activity only in the lungs. Both formulations are PEG-free (have 0 mol % PEG). The 3450 formulation has 45 mol % DOTAP and the 4750 formulation has 75 mol % DOTAP, as indicated in the summary at bottom. FIG. 7B shows that the average tdTomato signal levels observed in the lungs, liver, heart, and spleen among the mice tested did not significantly differ between the two formulations tested. The tdTomato signal production was not dose-dependent and can be described as always on-or-off. FIG. 7C shows the percentage of tdTomato signal in each mouse found in the lung, liver, heart, and spleen, and demonstrates that for both formulations, nearly 100% of tdTomato expression was in the lungs. FIG. 7D shows the average Cy7 (LNP localization) signal in the lungs, liver, heart, and spleen among the mice administered 3450 and 4750 formulations. FIG. 7E shows the average percent distribution of Cy7 radiance (LNP localization) in the lung, liver, heart, and spleen of mice administered 3450 and 4750 formulations. Notably, the 4750 formulation delivered higher levels of LNPs to the lung tissue than did the 3450 formulation.

FIG. 8 shows that DOTAP-LNPs can also be administered via intraarticular injections for efficient local intracellular delivery of nucleic acid cargo to the knee. Activities of the mFluc and mCre reporter systems in the knees of treated mice and rats are shown, which demonstrated the successful expression and integration of the mRNA cargo reporter systems delivered by DOTAP-LNPs injected into the local tissue area.

FIGS. 9A and 9B show that intratracheal administration of cargo-loaded DOTAP-LNPs also resulted in successful delivery of nucleic acid cargoes to the lung tissue. FIG. 9A shows that local delivery of DOTAP-LNPs to lungs was observed in healthy (Ail4 wild-type) mice when administered via intratracheal (topical) instillation. Time-dependent imaging at 6, 24, and 48 hours post-administration showed that local administration of mCre-loaded DOTAP-LNPs started to exhibit cargo nucleic acid expression-mediated effects in the lungs and trachea of treated subjects as early as 6 h post-administration. In addition, no off-target effects were observed in the spleen or liver of treated subjects. FIG. 9B shows immunohistochemistry-stained lung tissue sections that exhibit mCre mRNA cargo-loaded DOTAP-LNPs to have accessed key cell types via intratracheal (topical) instillation, even as early as at 6 h post-administration. In these lung tissue sections, macrophages, endothelial and epithelial cells are indicated by arrows. These results demonstrated that PEG-free DOTAP-LNPs could also be successfully used for local administration of nucleic acid cargoes into the lung, e.g., in clinical cases that require airway-associated cell activity.

### DETAILED DESCRIPTION OF THE INVENTION

The instant disclosure provides, at least in part, lipid particle compositions, formulations and associated methods, for delivery of lipid particle-associated molecular cargoes to the cells of a subject. In certain aspects, nucleic-acid lipid nanoparticles are provided that preferentially localize to and deliver associated nucleic acid cargoes to the lung of a subject, with delivery occurring to various types of tissue within the lung of a subject. DOTAP, a well-known quaternary amino lipid, is a structural component of the lipid nanoparticles (LNPs) disclosed herein that, upon systemic or local administration, without wishing to be bound by theory, appears to shift the tropism of LNP vectors disclosed herein specifically to lungs without requiring a further active-targeting component in the LNPs of the instant disclosure. Demonstrated herein is also the surprising structural affinity of DOTAP for lung tissues in mediating effective delivery of nucleic acid cargoes, in particular, expression of various reporter mRNAs, upon systemic administration (IV).

While fluorescently-labeled DOTAP LNPs of the instant disclosure were observed to accumulate not only in lung, but also in the liver of an injected subject at up to 25-40% of total LNPs, translation of an mRNA cargo to protein (and therefore intracellular activity) was only observed in lung tissues, in a manner that was identified as independent of the magnitude of the surface charge of the tested DOTAP-LNPs disclosed herein. Immunohistochemistry (IHC) evaluation of lung tissues also demonstrated endothelial cell, epithelial cell, fibroblast and macrophage cell delivery within lung tissues of mRNA cargoes using DOTAP LNPs of the instant disclosure. Moreover, effective DOTAP-based LNPs of the instant disclosure can be prepared while entirely eliminating PEG from the lipid formulations, which offers certain in vivo advantages for LNP-encapsulated therapeutics. Without wishing to be bound by theory, high positive charge of DOTAP was identified as likely sufficient to stabilize the particles via electrostatic stabilization, without requiring steric stabilization. The ability to avoid PEG in the formulation is another notable and surprising effect of the lipid particles of the instant disclosure, in that use of PEG-free compositions avoids the accelerated blood clearance (ABC) effect, a well-documented phenomenon caused by the body's immune system activating against PEG molecules on the surface of LNPs. ABC is responsible for the clearance of nanoparticles from systemic circulation upon repeated dosing. The instant disclosure, therefore, in certain embodiments, significantly enables/promotes repeated systemic administration of LNPs using DOTAP as a stabilizing lipid.

Nucleic acid therapy has well-known, tremendous potential to treat diseases at the gene level. However, safe and effective delivery systems are essential for nucleic acid therapeutics. Non-specific delivery to organs and tissues often results in off-site effects and toxicity. Delivery of therapeutics to a specific organ of interest is a well-recognized need in the development of lipid-nanoparticles, as well as in drug development generally. The concept of only targeting the cause of a disease without harming other parts of the body was described by Ehrlich 120 years ago. However, extant methods do not provide defined or well-known methodologies for developing nanoparticles targeting specific tissues without introducing additional ligand-based targeting strategies. Organ-specific targeting of lipid nanoparticles based on the structural affinity of the lipid to the tissue, as now disclosed herein, therefore meets a well-established need in terms of reducing off-site effects and toxicity.

Lungs are one of the key target organs for gene therapy. Specific delivery to the lungs by avoiding activity in the other organs is vital to treat respiratory system related diseases effectively. The instant disclosure demonstrates that incorporation of DOTAP, a well-known quaternary amino lipid shifts the tropism of vectors specifically to lungs without requiring an active-targeting component in the LNPs.

PEGylation of LNPs is believed to confer increased particle stability (both in vitro and in vivo). However, the instant disclosure provides exemplary compositions of DOTAP-based LNPs prepared without PEG in the formulation that are fully effective for intracellular delivery and activity of nucleic acid cargoes.

DOTAP is a positively charged lipid independent of the pH of the environment it is in, due to its quaternary amino structure. This contributes to the overall positive charge of the LNPs prepared with DOTAP as the main component. Although the current state of the art indicates that nanoparticle delivery to the lungs depends on lipid nanoparticles' high positive surface charge, introducing PEG can reduce the surface charge close to neutral or even negative levels, depending on the formulation. Without being bound by theory, it is hypothesized that DOTAP is a LNP component that is selectively taken up by lung tissues due to its structural properties, rather than solely due to its contribution to the surface charge of LNPs.

DOTAP LNP particle size and surface charge can be fine-tuned by modifying the N/P (N-to-P) ratio or molar composition of formulations. Therefore, a wide range of particles with specific physicochemical properties can be prepared for multiple applications, including local delivery by inhalation, intravenous or intra-articular delivery. Similarly, DOTAP can be used as the sole cationic lipid for nucleic acid encapsulation in the LNPs of the instant disclosure.

Lipid nanoparticles tend to remain within the blood compartment, as they are not able to extravasate across the continuous endothelial lining present in most blood vessels. At disease sites, however, the blood vessels may be leaky, allowing lipid nanoparticles extravasation and accumulation in the interstitial space. In tumors, for example, the immature neovasculature tends to exhibit pores or defects that can allow lipid nanoparticles of appropriate size to exit the blood vessels (Yuan et al., Cancer Research 54: 3352-3356, 1994). Similarly, at sites of infection or inflammation, the endothelial permeability barrier can be compromised, allowing lipid nanoparticles to accumulate in these regions. In contrast, the blood vessels present in most normal, healthy tissues tend to have continuous endothelial linings. Hence, lipid nanoparticles delivery can reduce drug exposure to these areas. Exceptions are the organs of the mononuclear phagocyte system (MIPS), such as the liver and spleen, where fenestrated capillaries are present. Although DOTAP LNPs have been identified to selectively deliver cargoes to lung tissue, in some aspects, delivery to regions of leaky or fenestrated capillaries, such as joint, inflammation sites, or the liver, with the DOTAP LNPs of the instant disclosure or variations thereof, is also contemplated.

Various expressly contemplated components of certain compositions and methods of the instant disclosure are considered in additional detail below.

### DOTAP-Based Lipid Nanoparticle Compositions

1,2-dioleoyl-3-trimethylammonium-propane, DOTAP, or 18:1 TAP is a cationic lipid. DOTAP is cationically charged independent of pH, due to its quaternary structure. The structure of DOTAP (C₄₂H₈₀NO₄⁺) is shown below:

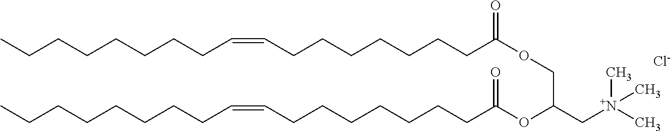 [SMILES: CCCCCCCC/C=C\CCCCCCCC(=O)OCC(C[N+](C)(C)C)OC(=O)CCCCCCC/C=C\CCCCCCCC ; [Cl-]]

In certain embodiments of the lipid particles of the instant disclosure, and in related methods of the instant disclosure, at least about 10%, at least about 20%, at least about 30%, at least about 40%, at least about 50%, at least about 60%, at least about 70%, at least about 80%, at least about 90%, at least about 95%, at least about 99%, or 100% (molar basis) of the total phospholipids present in a lipid nanoparticle of the disclosure are DOTAP. In certain embodiments of lipid nanoparticles of the instant disclosure, and the related methods of the instant disclosure, at least about 0.1%, at least about 5%, at least about 10%, at least about 20%, at least about 40%, at least about 60%, or at least about 80% (molar basis) of the total lipids are cholesterol. In certain embodiments at least about 0.1%, at least about 5%, at least about 10%, at least about 20%, at least about 40%, at least about 60%, or at least about 80% (molar basis) of the total lipids are other non-cationic lipids, e.g. DOPC, DSPC and/or DOPE.

Lipid nanoparticles of any size may be used according to the instant disclosure. In certain embodiments of the instant disclosure, lipid nanoparticles have a size ranging from about 0.02 microns to about 0.4 microns, between about 0.05 and about 0.2 microns, or between 0.07 and 0.12 microns in diameter.

In some embodiments, the LNPs may also comprise other cationic lipids including but not limited to, those comprising a protonatable tertiary amine (e.g., pH-titratable) head group; C18 alkyl chains, wherein each alkyl chain independently has 0 to 3 (e.g., 0, 1, 2, or 3) double bonds; and ether, ester, or ketal linkages between the head group and alkyl chains. Such cationic lipids include, but are not limited to, 1,2-distearyloxy-N,N-dimethyl-3-aminopropane (DSDMA), 1,2-dioleyloxy-N,N-dimethyl-3-aminopropane (DODMA), 1,2-dilinoleyloxy-N,N-dimethyl-3-aminopropane (DLinDMA), 1,2-dilinolenyloxy-N,N-dimethyl-3-aminopropane (DLenDMA), 1,2-di-γ-linolenyloxy-N,N-dimethylaminopropane (γ-DLenDMA, 1,2-dilinoleyloxy-keto-N,N-dimethyl-3-aminopropane (DLinK-DMA), 1,2-dilinoleyl-4-(2-dimethylaminoethyl)-[1,3]-dioxolane (DLinKC2-DMA) (also known as DLin-C2K-DMA, XTC2, and C2K), 2,2-dilinoleyl-4-(3-dimethylaminopropyl)[1,3]-dioxolane (DLin-K-C3-DMA), 2,2-dilinoleyl-4-(4-dimethylaminobutyl)[1,3]-dioxolane (DLin-K-C4-DMA), 1,2-dilinolenyloxy-4-(2-dimethylaminoethyl)-[1,3]-dioxolane (γ-DLen-C2K-DMA), 1,2-di-γ-linolenyloxy-4-(2-dimethylaminoethyl)-[1,3]-dioxolane (γ-DLen-C2K-DMA), dilinoleylmethyl-3-dimethylaminopropionate (DLin-M-C2-DMA) (also known as MC2), (6Z,9Z,28Z,31Z)-heptatriaconta-6,9,28,31-tetraen-19-yl 4-(dimethylamino) butanoate (DLin-M-C3-DMA) (also known as MC3) and 3-(dilinoleylmethoxy)-N,N-dimethylpropan-1-amine (DLin-MP-DMA) (also known as 1-B11).

In some embodiments LNPs may include neutral lipids, for example, diacylphosphatidylcholine, diacylphosphatidylethanolamine, ceramide, sphingomyelin, cephalin, cholesterol, cerebrosides and diacylglycerols. In other embodiments, LNPs may include anionic lipids, including but not limited to, phosphatidylglycerols, cardiolipins, diacylphosphatidylserines, diacylphosphatidic acids, N-dodecanoyl phosphatidylethanolamines, N-succinyl phosphatidylethanolamines, N-glutarylphosphatidylethanolamines, lysylphosphatidylglycerols, palmitoyloleyolphosphatidylglycerol (POPG), and other anionic modifying groups joined to neutral lipids. In some aspects, the non-cationic lipid used in the instant disclosure is 1,2-dioleoyl-sn-glycero-3-phosphocholine (DOPC), 1,2-Distearoyl-sn-glycero-3-phosphocholine (DSPC), and/or 1,2-Dioleoyl-sn-glycero-3-phosphoethanolamine (DOPE). In some aspects, the non-cationic lipid is cholesterol (CHE) and/or β-sitosterol.

In some embodiments that employ PEG-conjugated lipids, the PEG-conjugated lipid is one or more of a polyethyleneglycol (PEG)-lipid conjugate, a polyamide (ATTA)-lipid conjugate, and a mixture thereof. In one aspect, the PEG-lipid conjugate is one or more of a PEG-dialkyloxypropyl (DAA), a PEG-diacylglycerol (DAG), a PEG-phospholipid, a PEG-ceramide, and a mixture thereof. In one aspect, the PEG-DAG conjugate is one or more of a PEG-dilauroylglycerol (C12), a PEG-dimyristoylglycerol (C14), a PEG-dipalmitoylglycerol (C16), and a PEG-distearoylglycerol (Cis). In one aspect, the PEG-DAA conjugate is one or more of a PEG-dilauryloxypropyl (C₁₂), a PEG-dimyristyloxypropyl (C₁₄), a PEG-dipalmityloxypropyl (C₁₆), and a PEG-di stearyloxypropyl (Cis). In some embodiments, PEG is 2-dimyristoyl-rac-glycero-3-methoxypolyethylene glycol-2000 (PEG-DMG) and/or 1,2-distearoyl-rac-glycero-3-methoxypolyethylene glycol-2000 (PEG-DSG).

In some embodiments, amphipathic lipids are included in LNPs of the instant disclosure. Amphipathic lipids may refer to any suitable material, wherein the hydrophobic portion of the lipid material orients into a hydrophobic phase, while the hydrophilic portion orients toward the aqueous phase. Such compounds include, but are not limited to, phospholipids, aminolipids, and sphingolipids. Representative phospholipids include sphingomyelin, phosphatidylcholine, phosphatidylethanolamine, phosphatidylserine, phosphatidylinositol, phosphatidic acid, palmitoyloleoyl phosphatdylcholine, lysophosphatidylcholine, lysophosphatidylethanolamine, dipalmitoylphosphatidylcholine, dioleoylphosphatidylcholine, distearoylphosphatidylcholine, or dilinoleoylphosphatidylcholine. Other phosphorus-lacking compounds, such as sphingolipids, glycosphingolipid families, diacylglycerols, and β-acyloxyacids, can also be used. Additionally, such amphipathic lipids can be readily mixed with other lipids, such as triglycerides and sterols.

Also suitable for inclusion in the lipid particles of the instant disclosure are programmable fusion lipid formulations. Such formulations have little tendency to fuse with cell membranes and deliver their cargo until a given signal event occurs. This allows the lipid formulation to distribute more evenly after injection into an organism or disease site before it starts fusing with cells. The signal event can be, for example, a change in pH, temperature, ionic environment, or time. In the latter case, a fusion delaying or “cloaking” component, such as an ATTA-lipid conjugate or a PEG-lipid conjugate, can simply exchange out of the lipid nanoparticle membrane over time. By the time the formulation is suitably distributed in the body, it has lost sufficient cloaking agent so as to be fusogenic. With other signal events, it is desirable to choose a signal that is associated with the disease site or target cell, such as increased temperature at a site of inflammation.

In certain embodiments, it can be desirable to target the lipid nanoparticles of this disclosure further, using targeting moieties that are specific to a cell type or tissue. Targeting of lipid nanoparticles using a variety of targeting moieties, such as ligands, cell surface receptors, glycoproteins, vitamins (e.g., riboflavin) and monoclonal antibodies, has been previously described (see, e.g., U.S. Pat. Nos. 4,957,773 and 4,603,044). The targeting moieties can comprise the entire protein or fragments thereof.

Targeting mechanisms generally require that the targeting agents be positioned on the surface of the lipid nanoparticle in such a manner that the target moiety is available for interaction with the target, for example, a cell surface receptor. A variety of different targeting agents and methods are known and available in the art, including those described, e.g., in Sapra, P. and Allen, T M, Prog. Lipid Res. 42(5):439-62 (2003); and Abra, R M et al., J. Lipid nanoparticle Res. 12:1-3, (2002).

Standard methods for coupling target agents can be used. For example, phosphatidylethanolamine, which can be activated for attachment of target agents, or derivatized lipophilic compounds, such as lipid-derivatized bleomycin, can be used. Antibody-targeted lipid nanoparticles can be constructed using, for instance, lipid nanoparticles that incorporate protein A (see, Renneisen, et al., J. Bio. Chem., 265:16337-16342 (1990) and Leonetti, et al., Proc. NatL. Acad. Sci. (USA), 87:2448-2451 (1990). Other examples of antibody conjugation are disclosed in U.S. Pat. No. 6,027,726, the teachings of which are incorporated herein by reference. Examples of targeting moieties can also include other proteins, specific to cellular components, including antigens associated with neoplasms or tumors. Proteins used as targeting moieties can be attached to the lipid nanoparticles via covalent bonds (see, Heath, Covalent Attachment of Proteins to Lipid nanoparticles, 149 Methods in Enzymology 111-119 (Academic Press, Inc. 1987)). Other targeting methods include the biotin-avidin system.

A variety of methods for preparing lipid nanoparticles are known in the art, including e.g., those described in Szoka, et al., Ann. Rev. Biophys. Bioeng., 9:467 (1980); U.S. Pat. Nos. 4,186,183, 4,217,344, 4,235,871, 4,261,975, 4,485,054, 4,501,728, 4,774,085, 4,837,028, 4,946,787; PCT Publication No. WO 91/17424; Deamer and Bangham, Biochim. Biophys. Acta, 443:629-634 (1976); Fraley, et al., Proc. Natl. Acad. Sci. USA, 76:3348-3352 (1979); Hope, et al., Biochim. Biophys. Acta, 812:55-65 (1985); Mayer, et al., Biochim. Biophys. Acta, 858:161-168 (1986); Williams, et al., Proc. Natl. Acad. Sci., 85:242-246 (1988); Lipid nanoparticles, Marc J. Ostro, ed., Marcel Dekker, Inc., New York, 1983, Chapter 1; Hope, et al., Chem. Phys. Lip., 40:89 (1986); and Lipid nanoparticles: A Practical Approach, Torchilin, V. P. et al., ed., Oxford University Press (2003), and references cited therein. Suitable methods include, but are not limited to, sonication, extrusion, high pressure/homogenization, microfluidization, detergent dialysis, calcium-induced fusion of small lipid nanoparticle vesicles, and ether-infusion methods, all of which are well known in the art.

In some embodiments of the instant disclosure, DOTAP-based LNPs were prepared using a microfluidic mixing process. Briefly, lipid stocks of DOTAP, DOPC, CHE and PEG-DMG were prepared in ethanol at 20 mg/ml concentration. Different N/P ratios (2-6), PEGylation (0-2%), and lipid compositions (molar ratio between the lipids to each other) were investigated. In all formulations, the DOTAP mol percent was varied between 20-80 (in certain exemplified series of formulations, the DOTAP mol percent was kept at 45). Lipids were mixed together for the given compositions in ethanol with a final lipid concentration of 5.8 mg/ml. Firefly luciferase mRNA (mFluc) was used as the mRNA in the aqueous phase at a concentration of 0.25-2 mg/ml. The mixing of two phases and LNP preparation was performed using a 2:1 or 3:1 aqueous to organic volume ratio, and at an 8 or 12 ml/min flow rate in a microfluidic chip with staggered herringbone structure. Resulting LNPs were subjected to purification and buffer exchange by tangential flow filtration (TFF) against PBS. Alternatively, resulting LNPs were subjected to dialysis against PBS using a membrane with a MWCO range between 8-300 kDa. Table 1, below in Example 1, summarizes the characterization parameters of the formulations. Precise control of the characterization parameters enabled the preparation of DOTAP LNPs in the size range of 188-51 nm, surface charge between 0-26 mV, and PDI below 0.2. All formulations showed more than 98% of encapsulation efficiency (EE) calculated by Ribogreen assay using the manufacturer's protocol.

Lipid nanoparticles prepared according to methods as disclosed herein and as known in the art can in certain embodiments be stored for substantial periods of time prior to drug loading and administration to a patient. For example, lipid nanoparticles can be dehydrated, stored, and subsequently rehydrated and loaded with one or more active agents, prior to administration. Lipid nanoparticles may also be dehydrated after being loaded with one or more active agents. Dehydration can be accomplished by a variety of methods available in the art, including the dehydration and lyophilization procedures described, e.g., in U.S. Pat. Nos. 4,880,635, 5,578,320, 5,837,279, 5,922,350, 4,857,319, 5,376,380, 5,817,334, 6,355,267, and 6,475,517. In one embodiment, lipid nanoparticles are dehydrated using standard freeze-drying apparatus, i.e., they are dehydrated under low pressure conditions. Also, the lipid nanoparticles can be frozen, e.g., in liquid nitrogen, prior to dehydration. Sugars can be added to the LNP environment, e.g., to the buffer containing the lipid nanoparticles, prior to dehydration, thereby promoting the integrity of the lipid nanoparticle during dehydration. See, e.g., U.S. Pat. No. 5,077,056 or 5,736,155.

Lipid nanoparticles may be sterilized by conventional methods at any point during their preparation, including, e.g., after sizing or after generating a pH gradient.

### Cargo-Loaded Lipid Particle Compositions

In various embodiments, lipid particles of the instant disclosure may be used for many different applications, including the delivery of an active agent to a cell, tissue, organ or subject. For example, lipid nanoparticles of the instant disclosure may be used to deliver a therapeutic agent systemically via the bloodstream or to deliver a cosmetic agent to the skin. Accordingly, lipid nanoparticles of the instant disclosure and one or more active agents as cargo(es) are included in the instant disclosure.

### Lipid Particle Cargoes

The instant disclosure describes lipid nanoparticles (i.e., a lipid nanoparticle comprising DOTAP) in combination with an active agent as a cargo. Active agents, as used herein, include any molecule or compound capable of exerting a desired effect on a cell, tissue, organ, or subject. Such effects may be biological, physiological, or cosmetic, for example. Active agents may be any type of molecule or compound, including e.g., nucleic acids, such as single- or double-stranded polynucleotides, plasmids, antisense RNA, RNA interference agents, including, e.g., DNA-DNA hybrids, DNA-RNA hybrids, RNA-DNA hybrids, RNA-RNA hybrids, short interfering RNAs (siRNA), micro RNAs (mRNA) and short hairpin RNAs (shRNAs); peptides and polypeptides, including, e.g., antibodies, such as, e.g., polyclonal antibodies, monoclonal antibodies, antibody fragments; humanized antibodies, recombinant antibodies, recombinant human antibodies, and Primatized™ antibodies, cytokines, growth factors, apoptotic factors, differentiation-inducing factors, cell surface receptors and their ligands; hormones; and small molecules, including small organic molecules or compounds.

Nucleic acids associated with or encapsulated by LNPs may contain modifications including but not limited to those selected from the following group: 2′-O-methyl modified nucleotides, a nucleotide comprising a 5′-phosphorothioate group, a terminal nucleotide linked to a cholesteryl derivative, a 2′-deoxy-2′-fluoro modified nucleotide, a 5′-methoxy-modified nucleotide (e.g., 5′-methoxyuridine), a 2′-deoxy-modified nucleotide, a locked nucleotide, an abasic nucleotide, a 2′-amino-modified nucleotide, a 2′-alkyl-modified nucleotide, a morpholino nucleotide, a phosphoramidate, a non-natural base comprising nucleotide; internucleoside linkages or backbones including phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2.′

In certain embodiments, the active agent is a mRNA or a vector capable expressing a mRNA in a cell.

In embodiments, the active agent is a CRISPR/Cas system. Optionally, an LNP of the instant disclosure can be formulated to include, e.g., both a guide strand (gRNA) and a Cas enzyme as cargoes, thereby providing a self-contained delivery vehicle capable of effecting and controlling CRISPR-mediated targeting of a gene in a target cell.

In certain featured embodiments, the active agent is a nucleic acid modulating controller (e.g., a mRNA that encodes protein controller components, as described above).

In some embodiments, the active agent is a therapeutic agent, or a salt or derivative thereof. Therapeutic agent derivatives may be therapeutically active themselves or they may be prodrugs, which become active upon further modification. Thus, in one embodiment, a therapeutic agent derivative retains some or all of the therapeutic activity as compared to the unmodified agent, while in another embodiment, a therapeutic agent derivative lacks therapeutic activity.

In various embodiments, therapeutic agents include agents and drugs, such as anti-inflammatory compounds, narcotics, depressants, anti-depressants, stimulants, hallucinogens, analgesics, antibiotics, birth control medication, antipyretics, vasodilators, anti-angiogenics, cytovascular agents, signal transduction inhibitors, vasoconstrictors, hormones, and steroids.

In certain embodiments, the active agent is an oncology drug, which may also be referred to as an anti-tumor drug, an anti-cancer drug, a tumor drug, an antineoplastic agent, or the like. Examples of oncology drugs that may be used according to the instant disclosure include, but are not limited to, adriamycin, alkeran, allopurinol, altretamine, amifostine, anastrozole, araC, arsenic trioxide, azathioprine, bexarotene, biCNU, bleomycin, busulfan intravenous, busulfan oral, capecitabine (Xeloda), carboplatin, carmustine, CCNU, celecoxib, chlorambucil, cisplatin, cladribine, cyclosporin A, cytarabine, cytosine arabinoside, daunorubicin, cytoxan, daunorubicin, dexamethasone, dexrazoxane, dodetaxel, doxorubicin, doxorubicin, DTIC, epirubicin, estramustine, etoposide phosphate, etoposide and VP-16, exemestane, FK506, fludarabine, fluorouracil, 5-FU, gemcitabine (Gemzar), gemtuzumab-ozogamicin, goserelin acetate, hydrea, hydroxyurea, idarubicin, ifosfamide, imatinib mesylate, interferon, irinotecan (Camptostar, CPT-111), letrozole, leucovorin, leustatin, leuprolide, levamisole, litretinoin, megastrol, melphalan, L-PAM, mesna, methotrexate, methoxsalen, mithramycin, mitomycin, mitoxantrone, nitrogen mustard, paclitaxel, pamidronate, Pegademase, pentostatin, porfimer sodium, prednisone, rituxan, streptozocin, STI-571, tamoxifen, taxotere, temozolamide, teniposide, VM-26, topotecan (Hycamtin), toremifene, tretinoin, ATRA, valrubicin, velban, vinblastine, vincristine, VP16, and vinorelbine. Other examples of oncology drugs that may be used according to the instant disclosure are ellipticin and ellipticin analogs or derivatives, epothilones, intracellular kinase inhibitors and camptothecins.

While LNP compositions of the instant disclosure generally comprise a single active agent, in certain embodiments, they may comprise more than one active agent.

In other embodiments of the instant disclosure, the lipid nanoparticles of the instant disclosure have a plasma circulation half-life of at least 0.5, 0.8, 1.2, 1.5, 2.0, 4.0, 6.0, 8.0, or 12 hours. In some embodiments, lipid nanoparticles have a plasma drug half-life of at least 0.5, 0.8, 1.2, 1.5, 2.0, 4.0, 6.0, 8.0, or 12 hours. Circulation and blood or plasma clearance half-lives may be determined as described, for example, in U.S. Patent Publication No. 2004-0071768-A1.

The instant disclosure also provides lipid nanoparticles and variations thereof in kit form. The kit may comprise a ready-made formulation or a formulation that requires mixing before administration. The kit will typically comprise a container that is compartmentalized for holding the various elements of the kit. The kit will contain the lipid nanoparticle compositions of the instant disclosure or the components thereof, in hydrated or dehydrated form, with instructions for their rehydration and administration. In particular embodiments, a kit comprises at least one compartment containing a lipid nanoparticle of the instant disclosure that is loaded with an active agent. In another embodiment, a kit comprises at least two compartments, one containing a lipid nanoparticle of the instant disclosure and the other containing an active agent. Of course, it is understood that any of these kits may comprise additional compartments, e.g., a compartment comprising a buffer, such as those described in U.S. Patent Publication No. 2004-0228909-A1. Kits of the instant disclosure, which comprise lipid nanoparticles comprising DOTAP, may also contain other features of the kits described in U.S. Patent Publication No. 2004-0228909 A1. Further the kit may contain drug-loaded lipid nanoparticles in one compartment and empty lipid nanoparticles in a second compartment. Alternatively, the kit may contain a lipid nanoparticle of the instant disclosure, an active agent to be loaded into the lipid nanoparticle of the instant disclosure in a second compartment, and an empty lipid nanoparticle in a third compartment.

In a particular embodiment, a kit of the instant disclosure comprises a therapeutic compound encapsulated in a lipid nanoparticle comprising DOTAP, where DOTAP constitutes at least 20%, at least 50%, or at least 70% (molar basis) of total phospholipids present in the lipid nanoparticle, as well as an empty lipid nanoparticle. In one embodiment, the lipid nanoparticle containing therapeutic compound and the empty lipid nanoparticle are present in different compartments of the kit.

Efficacy of Lipid Particle-Mediated Cargo Delivery

In certain embodiments, the instant disclosure is based, at least in part, upon the surprising result that from 25-75% (mol/weight) DOTAP-based lipid particles are highly effective at delivering active nucleic acid cargoes into cells of the lungs, relative to other tissues. Further, reporter activity of the encapsulated active agent (cargo), e.g. mRNA, occurred almost exclusively in the lung tissue, as compared to other tissues. The efficacy of localization of a lipid particle may be described as the fold difference (increase or decrease) in localization of the nucleic acid-lipid particle to a particular tissue of the subject relative to that of one or more other tissues of the subject. The efficacy of activity as a further component in assessing delivery may be described as the fold difference (increase or decrease) in activity of the active agent, e.g., a nucleic acid cargo or other compound, within cells of a particular tissue of the subject, relative to that observed in cells of one or more other tissues of the subject. In some embodiments, the fold difference may therefore be detected at the cellular level, or can be detected by appropriate proxy for events occurring at the cellular level. In some embodiments, the cell of the lung tissue affected is one or more of epithelium, endothelium, interstitial connective tissue, blood vessel, hematopoietic tissue, lymphoid tissue, and pleura. In some embodiments, the fold-difference in effect/activity may be detected at a sub-cellular level, i.e. where activity is detectible in the nuclei of targeted cells.

To determine the efficacy of localization of the LNP, assays may be performed according to the characteristics of the labeled or detected molecule of interest. In illustrative embodiments of the instant disclosure, a fluorescently labeled lipid has been used to determine LNP localization. In other embodiments, a labeled peptide, or other component of a lipid particle may be used. In some embodiments, the localization is detectible in individual cells. In some embodiments the label is a fluorescent label, i.e. a fluorescently labeled lipid such as Cy7. In other embodiments the label of the lipid nanoparticle may be a quantum dot, or the lipid detectible by stimulated Raman scattering. In other embodiments the label is any fluorophore known in the art, i.e. with excitation and emission in the ultraviolet, visible, or infrared spectra. In some embodiments the localization is detected or further corroborated by immunohistochemistry or immunofluorescence methods.

The efficacy of localization may be described as the fold difference (increase or decrease) in localization of the nucleic acid-lipid particle to a tissue, i.e. lung tissue, of the subject relative to one or more other tissues of the subject. In illustrative embodiments of the instant disclosure, Cy7 labeled lipids were imaged in vivo and fluorescence radiance served as an indication of Cy7-LNP concentration (see Example 3 below). The Cy7 labeled DOTAP nucleic acid LNPs exhibited increased efficacy of localization to the lungs relative to other tissues, in particular the heart, spleen, ovaries and pancreas. In some embodiments of the instant disclosure a Cy7 labeled nucleic acid LNP, exhibited at least two-fold localization to the lungs relative to the heart, spleen, ovaries or pancreas. In some embodiments, a Cy7 labeled nucleic acid LNP, exhibited at least three-fold localization to the lungs, in some embodiments a Cy7 labeled nucleic acid LNP, exhibited at least four-fold localization to the lungs, in some embodiments the Cy7 labeled nucleic acid LNP, exhibited at least five-fold localization to the lungs, in some embodiments the Cy7 labeled a nucleic acid LNP, exhibited at least six-fold localization to the lungs, relative to that of the heart, spleen, ovaries and pancreas.

To determine the efficacy of activity of the active agent encapsulated by the LNP, assays may be performed according to the characteristics of the active agent. In certain embodiments, the active agent in the DOTAP LNP is a nucleic acid. In other embodiments, the active agent in the DOTAP LNP is a small molecule or other compound.

In some embodiments, the active agent in the DOTAP-based LNP is a mRNA. In illustrative embodiments, the localized expression of a reporter mRNA, i.e. luciferase, served as an indication of intracellular delivery efficacy for an mRNA as the active agent/cargo. In other embodiments, the mRNA may encode Cre enzyme, green fluorescent protein, red fluorescent protein, yellow fluorescent protein or blue fluorescent protein. Or in therapeutic embodiments, the mRNA may encode for a protein for therapeutic intracellular expression in LNP-targeted cells of a subject, optionally where intracellular levels of delivered mRNA or encoded protein can be detected by methods known in the art, as appropriate for the therapeutic mRNA that is delivered. In other embodiments, a reporter mRNA encodes a cell surface marker, such as a Lyt2 cell surface marker. In still other embodiments, the reporter can be a β-galactosidase, α-lactamase, an alkaline phosphatase or a horse-radish peroxidase. In other embodiments, the reporter mRNA encodes a negative selection marker, such as thymidine kinase (tk), HRPT or APRT. In some embodiments, immunohistochemistry or immunofluorescence is used to detect or corroborate activity of the reporter mRNA.

In certain embodiments, the effectiveness of a lipid particle of the instant disclosure in delivering a cargo is assessed based upon the levels of activity observed for the cargo (active agent) intracellularly within a lipid particle-targeted tissue. Such effects can be identified as fold-differences in activity, as compared to an appropriate control formulation and/or tissue, e.g., the delivery efficacy of a LNP with nucleic acid cargo may be described as the fold difference (increase or decrease) in activity of the nucleic acid cargo in cells of a targeted tissue, i.e. the lung tissue, of a subject relative to one or more other tissues of the subject. Thus, for certain nucleic acid cargoes, delivery efficacy of an LNP formulation can be identified as a LNP that achieves, e.g., two-fold greater intracellular activity of the nucleic acid payload in targeted tissue cells than in non-targeted tissue cells, or relative to a LNP formulation that does not include the nucleic acid cargo. Optionally, an effective LNP formulation for delivery of a nucleic acid cargo can be described as one that achieves at least about a three-fold greater, optionally about a four-fold greater, optionally about a five-fold greater, optionally about a six-fold greater, optionally about a seven-fold greater, optionally about a eight-fold greater, optionally about a nine-fold greater, optionally about a ten-fold greater, optionally about a 50-fold greater, optionally about a 100-fold greater, etc. intracellular activity of the nucleic acid payload in targeted tissue cells than in non-targeted tissue cells, or relative to a LNP formulation that does not include the nucleic acid cargo. In illustrative embodiments of the instant disclosure, DOTAP LNPs delivered a luciferase mRNA that was expressed in cells of the lung tissue of the subject at a level that was significantly higher than that of cells of the liver, heart, spleen, ovary, pancreas and kidney of the subject (see Example 3 below). Luciferin was delivered intravenously to the subject, and the cells expressing luciferase were detected through in vivo bioluminescence imaging. In some embodiments, the luciferase mRNA was expressed in cells of the lung tissue of the subject at a level that was at least two-fold higher than expression of the mRNA in cells of the liver, heart, spleen, ovary, pancreas and kidney of the subject. In some embodiments, cargo mRNA was expressed in cells of the lung tissue of the subject at a level that was at least three-fold the higher than expression of the mRNA in cells of the liver, heart, spleen, ovary, pancreas and kidney of the subject. In some embodiments, the luciferase mRNA was expressed at least four-fold higher in the lungs, in some embodiments, the luciferase mRNA was expressed at least five-fold higher in the lungs, in some embodiments at least six-fold higher in the lungs, in some embodiments at least seven-fold higher in the lungs, in some embodiments at least eight-fold higher in the lungs, in some embodiments at least nine-fold higher in the lungs, in some embodiments at least ten-fold higher in the lungs, in some embodiments at least eleven-fold higher in the lungs, in some embodiments at least twelve-fold higher in the lungs, in some embodiments at least thirteen-fold higher in the lungs, in some embodiments at least fourteen-fold higher in the lungs, in some embodiments at least fifteen-fold higher in the lungs, in some embodiments at least twenty-fold higher in the lungs, than expression of the cargo mRNA in cells of the liver, heart, spleen, ovary, pancreas and kidney of the subject.

In other embodiments, DOTAP LNPs can be employed to deliver a RNAi agent (e.g., a siRNA) to a tissue, i.e. a lung tissue. For siRNA or other RNAi agents, delivery and activity efficacy measurements can employ, for example, target-specific PCR to detect transcript levels, immunosorbent or other immunological methods to detect target protein levels, and/or Flow Cytometry (FACS) (Testoni et al., Blood 1996, 87:3822.). In some embodiments, a siRNA may be active in cells of the lung tissue of the subject at a level that is at least two-fold higher than in cells of the liver, heart, spleen, ovary, pancreas and kidney of the subject. In some embodiments, an siRNA may be active in cells of the lung tissue of the subject at a level that is at least three-fold higher than in cells of the liver, heart, spleen, ovary, pancreas and kidney of the subject. In some embodiments, the siRNA may be active at a level at least four-fold higher in the lungs, in some embodiments, the siRNA may be active at a level at least five-fold higher in the lungs, in some embodiments the siRNA may be active at a level at least six-fold higher in the lungs, in some embodiments at least seven-fold higher in the lungs, in some embodiments at least eight-fold higher in the lungs, in some embodiments at least nine-fold higher in the lungs, in some embodiments at least ten-fold higher in the lungs, in some embodiments at least eleven-fold higher in the lungs, in some embodiments at least twelve-fold higher in the lungs, in some embodiments at least thirteen-fold higher in the lungs, in some embodiments at least fourteen-fold higher in the lungs, in some embodiments at least fifteen-fold higher in the lungs, in some embodiments at least twenty-fold higher in the lungs, etc., than activity of the siRNA in cells of the liver, heart, spleen, ovary, pancreas and kidney of the subject. In related embodiments, a LNP that delivers a RNAi cargo preferentially to the lungs may exhibit, e.g., greater than 20% reduction in target transcript and/or protein levels in cells of targeted lung tissue, as compared to cells of non-targeted tissues or as compared to some other appropriate control (e.g., levels of target transcript in untreated lung tissue cells). Optionally, a LNP that delivers a RNAi cargo preferentially to the lungs may exhibit, e.g., more than 30% reduction, more than 40% reduction, more than 50% reduction, more than 60% reduction, more than 70% reduction, more than 80% reduction, more than 90% reduction, more than 95% reduction, more than 97% reduction, more than 97% reduction, more than 98% reduction or more than 99% reduction in target transcript and/or protein levels in cells of targeted lung tissue, as compared to cells of non-targeted tissues or as compared to some other appropriate control (e.g., levels of target transcript in untreated lung tissue cells).

In some embodiments, DOTAP-based LNPs of the instant disclosure can be used to deliver a CRISPR-Cas9 system to a tissue, i.e. a lung tissue. CRISP-Cas9 delivery and activity efficacy measurements may require, for example, PCR to detect Cas9, the genomic structures of targeted regions and/or target transcript levels, immunosorbent or other immunological methods to detect Cas9 or knock-in, knock-out, or other modifications of target proteins, and/or Flow Cytometry (FACS) (Testoni et al., Blood 1996, 87:3822.). In some embodiments, CRISP-Cas9-mediated effects may be identified in cells of the lung tissue of the subject at a level that is at least two-fold higher than in cells of the liver, heart, spleen, ovary, pancreas and kidney of the subject. In some embodiments, CRISP-Cas9-mediated effects may be identified in cells of the lung tissue of the subject at a level that is at least three-fold higher than in cells of the liver, heart, spleen, ovary, pancreas and kidney of the subject. In some embodiments, CRISP-Cas9-mediated effects may be identified in cells at a level at least four-fold higher in the lungs, in some embodiments, CRISP-Cas9-mediated effects may be identified in cells at a level at least five-fold higher in the lungs, in some embodiments, CRISP-Cas9-mediated effects may be identified in cells at a level at least six-fold higher, in some embodiments, CRISP-Cas9-mediated effects may be identified in cells at least seven-fold higher, in some embodiments at least eight-fold higher, in some embodiments at least nine-fold higher, in some embodiments at least ten-fold higher, in some embodiments at least eleven-fold higher, in some embodiments at least twelve-fold higher, in some embodiments at least thirteen-fold higher, in some embodiments at least fourteen-fold higher, in some embodiments at least fifteen-fold higher, in some embodiments at least twenty-fold higher, than CRISP-Cas9-mediated effects in cells of the liver, heart, spleen, ovary, pancreas and/or kidney of the subject.

In other embodiments, DOTAP LNPs may deliver an mRNA or other nucleic acid cargo to a tissue, i.e. a lung tissue, where expression and possibly activity occurs in the nucleus. In the instant disclosure, some embodiments have utilized the Cre recombinase enzyme as a reporter for nuclear activity of the active agent (see Example 6 below). The Cre recombinase enzyme requires translocation of the encoded protein to the nucleus and thus can serve as a reporter of nuclear translocation. The Cre recombinase catalyzes site-specific recombination of DNA between loxP sites. Upon Cre recombinase activity expression, due to loxP recombination, reporter fluorescent proteins are expressed. In one embodiment, the Ail4 mouse line used a Cre reporter loxP-flanked STOP cassette preventing transcription of a CAG promoter-driven red fluorescent protein variant (tdTomato), inserted into the Gt(ROSA)26S or locus. The Ail4 mice were intravenously injected with mCre-loaded DOTAP-LNPs and began expressing robust tdTomato fluorescence in the nucleic of lung cells following delivery and expression of the Cre enzyme, nuclear translocation of the Cre enzyme, and subsequently, Cre-mediated recombination of the tdTomato promoter. In exemplary embodiments, the efficacy of activity, ie. the expression of an mRNA detectible in the nucleus, was observable in the nucleus of lung cells at a level at least two-fold higher than that of cells in the liver, heart, and spleen. In some embodiments, the expression of an mRNA detectible in the nucleus was observable in the nucleus of lung cells at a level at least three-fold higher than that of cells in the liver, heart, and spleen. In some embodiments, the expression of an mRNA detectible in the nucleus was observable in the nucleus of lung cells at a level at least four-fold higher, in some embodiments the level was five-hold higher, in some embodiments, six-fold higher, in some embodiments, seven-fold higher, in some embodiments, eight-fold higher, in some embodiments, nine-fold higher, in some embodiments, ten-fold higher, in some embodiments, eleven-fold higher, in some embodiments, twelve-fold higher, in some embodiments, thirteen-fold higher, in some embodiments, fourteen-fold higher, in some embodiments, fifteen-fold higher, in some embodiments, twenty-fold higher, than activity of the mRNA detectible in the nucleus in cells of the liver, heart, and spleen.

In other embodiments, DOTAP LNPs may deliver small molecules or other compounds to a tissue, i.e. a lung tissue. The efficacy of localization or activity of small molecules may be determined by a number of in vivo imaging methods (e.g. PET/CT), mass spectrometry, as well as immunohistochemistry and immunofluorescence of target effects. In some embodiments, the DOTAP-LNP mediated localization and/or activity of the small molecule in the lung may be two-fold higher than that of other tissues, for example than that of the heart, spleen, ovary, and pancreas, optionally than that of the liver and/or kidney. In some embodiments, the DOTAP-LNP mediated localization and/or activity of the small molecule in the lung may be three-fold higher than that of other tissues, four-fold higher, five-hold higher, six-fold higher, seven-fold higher, eight-fold higher, nine-fold higher, ten-fold higher, eleven-fold higher, twelve-fold higher, thirteen-fold higher, fourteen-fold higher, fifteen-fold higher, or twenty-fold higher than that of other tissues, for example than that of the heart, spleen, ovary, and pancreas, optionally than that of the liver and/or kidney.

In certain embodiments, a lipid particle that is formulated for lung delivery refers to a lipid particle that exhibits preferential localization and intracellular delivery (based upon assessment of intracellular activity either directly or by proxy) of a cargo to lung cells, as compared to cells of one or more other tissues of a subject. For example, a lipid particle for lung delivery is one capable of inducing at least two-fold greater activity of a cargo (e.g., a nucleic acid cargo, e.g., a mRNA, a CRISPR/Cas system, a nucleic acid modulating controller, etc.) in lung cells of a subject, than in other tissues of the subject. Such effects in lung cells of a subject can be evaluated within one or more cell types of the lung, as described elsewhere herein. In certain embodiments, a lipid particle for lung delivery is one capable of inducing at least three-fold greater, at least four-fold greater, at least five-fold greater, at least six-fold greater, at least seven-fold greater, at least eight-fold greater, at least nine-fold greater, at least ten-fold greater, at least fifteen-fold greater, at least twenty-fold greater, at least thirty-fold greater, at least 40-fold greater, at least 50-fold greater, at least 60-fold greater, at least 70-fold greater, at least 80-fold greater, at least 90-fold greater, at least 100-fold greater, at least 1000-fold greater, etc. activity of a cargo (e.g., a nucleic acid cargo, e.g., a mRNA, a CRISPR/Cas system, a nucleic acid modulating controller, etc.) in lung cells of a subject, than in other tissues of the subject.

In still other embodiments, DOTAP LNPs formulated without PEG modified lipids may diminish or avoid the accelerated blood clearance effect (ABC), where the immune system targets PEG for removal. DOTAP notably provides sufficient stabilization to LNPs wherein PEGylated lipids are not required (see Example 1 below). Avoidance of ABC enhances the efficacy of activity of the DOTAP LNP active agent by enhancing the effects of subsequent doses of the LNPs. The efficacy of the DOTAP LNP's avoidance of accelerated blood clearance, relative to PEG containing formulations, may be determined by measuring the ABC of a DOTAP PEG-free formula relative to that of a DOTAP PEG-containing formula (0.5-1.5%) or, similarly, to that of other PEG-containing LNPs. The DOTAP PEG-free formula may retain LNPs in the blood and/or tissue upon the second or more dosing at a level at least two-fold, three-fold, four-fold, five-hold, six-fold, seven-fold, eight-fold, nine-fold, ten-fold, eleven-fold, twelve-fold, thirteen-fold, fourteen-fold, fifteen-fold, or at least twenty-fold greater than that of PEG-containing LNP compositions.

### LNP-Mediated Cargo Delivery

The lipid particle compositions disclosed herein can be used for a variety of purposes, including the delivery of an active agent or therapeutic agent or compound to a subject or patient in need thereof. Subjects include both humans and non-human animals. In certain embodiments, subjects are mammals. In other embodiments, subjects are one or more particular species or breed, including, e.g., humans, mice, rats, dogs, cats, cows, pigs, sheep, or birds.

Thus, the instant disclosure also provides methods of treatment for a variety of diseases and disorders, as well as methods intended to provide a cosmetic benefit.

Methods of Treatment

The LNP compositions of the instant disclosure may be used to treat any of a wide variety of diseases or disorders, including, but not limited to, inflammatory diseases, cardiovascular diseases, nervous system diseases, tumors, demyelinating diseases, digestive system diseases, endocrine system diseases, reproductive system diseases, hemic and lymphatic diseases, immunological diseases, mental disorders, muscoloskeletal diseases, neurological diseases, neuromuscular diseases, metabolic diseases, sexually transmitted diseases, skin and connective tissue diseases, urological diseases, and infections.

In certain embodiments, the LNP compositions can be employed to treat or prevent a lung disease or disorder, including but not limited to a disease or disorder selected from the following: lung cancer, pneumonia, pulmonary fibrosis, COPD, asthma, bronchiectasis, sarcoidosis, pulmonary hypertension, emphysema, alpha-1 antitrypsin deficiency, aspergillosis, bronchiolitis, bronchitis, pneumoconiosis, Coronaviruses, Middle Eastern Respiratory Syndrome, Severe Acute Respiratory Syndrome, cystic fibrosis, Legionairre's disease, influenza, pertussis, pulmonary embolism, and tuberculosis.

In other embodiments, the LNP compositions of the instant disclosure can be used to treat or prevent a joint disease or disorder, including but not limited to a disease or disorder selected from the following: rheumatoid arthritis, psoriatic arthritis, gout, tendinitis, bursitis, Carpal Tunnel Syndrome, and osteoarthritis.

In other embodiments, the LNP compositions of the instant disclosure can be used to treat or prevent an inflammatory disease or disorder, including but not limited to a disease or disorder selected from the following: inflammatory bowel disease, peritonitis, osteomyelitis, cachexia, pancreatitis, trauma induced shock, bronchial asthma, allergic rhinitis, cystic fibrosis, acute bronchitis, acute intense bronchitis, osteoarthritis, rheumatoid arthritis, infectious arthritis, post-infectious arthritis, gonocoele arthritis, tuberculous arthritis, arthritis, osteoarthritis, gout, spondyloarthropathies, ankylosing spondylitis, arthritis associated with vasculitis syndrome, nodular polyarteritis nervosa, irritable vasculitis, rugenic granulomatosis, rheumatoid polyposis myalgia, arthritis cell arteritis, calcium polycystic arthropathy, caustic gout, non-arthritic rheumatism, bursitis, hay fever, suppurative inflammation (e.g., tennis elbow), neuropathic joint disease, hemarthrosic, Henoch-Schlein purpura, hypertrophic osteoarthritis, multisized hemorrhoids, scoliosis, hemochromatosis, hyperlipoproteinemia, hypogammaglobulinemia, COPD, acute respiratory distress syndrome, acute lung injury, broncho-pulmonary dysplasia and systemic lupus erythematosus (SLE).

In other embodiments, the LNP compositions of the instant disclosure can be used to treat or prevent an epidermal disease or disorder, including but not limited to psoriasis, atopic dermatitis, scleroderma, eczema, rosacea, seborrheic dermatitis, melanoma, solar keratosis, ichthyosis, Grover's disease, common warts, keratoacanthoma, and seborrhoeic keratosis.

In one embodiment, the LNP compositions of the instant disclosure can be used to treat or prevent a type of cancer. In particular, these methods can be applied to cancers of the blood and lymphatic systems, including lymphomas, leukemia, and myelomas. Examples of specific cancers that may be treated according to the instant disclosure include, but are not limited to, Hodgkin's and non-Hodgkin's Lymphoma (NHL), including any type of NHL as defined according to any of the various classification systems such as the Working formulation, the Rappaport classification and, preferably, the REAL classification. Such lymphomas include, but are not limited to, low-grade, intermediate-grade, and high-grade lymphomas, as well as both B-cell and T-cell lymphomas. Included in these categories are the various types of small cell, large cell, cleaved cell, lymphocytic, follicular, diffuse, Burkitt's, Mantle cell, NK cell, CNS, AIDS-related, lymphoblastic, adult lymphoblastic, indolent, aggressive, transformed and other types of lymphomas. The methods of the instant disclosure can be used for adult or childhood forms of lymphoma, as well as lymphomas at any stage, e.g., stage I, II, III, or IV. The various types of lymphomas are well known to those of skill, and are described, e.g., by the American Cancer Society (see, e.g., www3.cancer.org).

The compositions and methods described herein may also be applied to any form of leukemia, including adult and childhood forms of the disease. For example, any acute, chronic, myelogenous, and lymphocytic form of the disease can be treated using the methods of the instant disclosure. In preferred embodiments, the methods are used to treat Acute Lymphocytic Leukemia (ALL). More information about the various types of leukemia can be found, inter alia, from the Leukemia Society of America (see, e.g., www.leukemia.org).

Additional types of tumors can also be treated using the methods described herein, such as neuroblastomas, myelomas, prostate cancers, small cell lung cancer, colon cancer, ovarian cancer, non-small cell lung cancer, brain tumors, breast cancer, and others.

The LNP compositions of the instant disclosure may be administered as first line treatments or as secondary treatments. In addition, they may be administered as a primary chemotherapeutic treatment or as adjuvant or neoadjuvant chemotherapy. For example, treatments of relapsed, indolent, transformed, and aggressive forms of non-Hodgkin's Lymphoma may be administered following at least one course of a primary anti-cancer treatment, such as chemotherapy and/or radiation therapy.

Administration of LNP Compositions

LNP compositions of the instant disclosure are administered in any of a number of ways, including parenteral, intravenous, systemic, local, oral, intratumoral, intramuscular, subcutaneous, intraperitoneal, inhalation, or any such method of delivery. In one embodiment, the compositions are administered parenterally, i.e., intraarticularly, intravenously, intraperitoneally, subcutaneously, or intramuscularly. In a specific embodiment, the LNP compositions are administered by intravenous infusion or intraperitoneally by a bolus injection. For example, in one embodiment, a patient is given an intravenous infusion of the lipid nanoparticle-encapsulated active agent through a running intravenous line over, e.g., 5-10 minutes, 15-20 minutes, 30 minutes, 60 minutes, 90 minutes, or longer. In one embodiment, a 60 minute infusion is used. In other embodiments, an infusion ranging from 6-10 or 15-20 minutes is used. Such infusions can be given periodically, e.g., once every 1, 3, 5, 7, 10, 14, 21, or 28 days or longer, preferably once every 7-21 days, and preferably once every 7 or 14 days.

LNP compositions of the instant disclosure may be formulated as pharmaceutical compositions suitable for delivery to a subject. The pharmaceutical compositions of the instant disclosure will often further comprise one or more buffers (e.g., neutral buffered saline or phosphate buffered saline), carbohydrates (e.g., glucose, mannose, sucrose, dextrose or dextrans), mannitol, proteins, polypeptides or amino acids such as glycine, antioxidants, bacteriostats, chelating agents such as EDTA or glutathione, adjuvants (e.g., aluminum hydroxide), solutes that render the formulation isotonic, hypotonic or weakly hypertonic with the blood of a recipient, suspending agents, thickening agents and/or preservatives. Alternatively, compositions of the instant disclosure may be formulated as a lyophilizate.

The concentration of drug and lipid nanoparticles in the pharmaceutical formulations can vary widely, i.e., from less than about 0.05%, usually at or at least about 2-5% to as much as 10 to 30% by weight and will be selected depend upon the particular drug used, the disease state being treated and the judgment of the clinician taking. Further, the concentration of drug and lipid nanoparticles will also take into consideration the fluid volume administered, the osmolality of the administered solution, and the tolerability of the drug and lipid nanoparticles. In some instances, it may be preferable to use a lower drug or lipid nanoparticle concentration to reduce the incidence or severity of infusion-related side effects.

Suitable formulations for use in the instant disclosure can be found, e.g., in Remington's Pharmaceutical Sciences, Mack Publishing Company, Philadelphia, Pa., 17^{th} Ed. (1985). Often, intravenous compositions will comprise a solution of the lipid nanoparticles suspended in an acceptable carrier, such as an aqueous carrier. Any of a variety of aqueous carriers can be used, e.g., water, buffered water, 0.4% saline, 0.9% isotonic saline, 0.3% glycine, 5% dextrose, and the like, and may include glycoproteins for enhanced stability, such as albumin, lipoprotein, globulin, etc. Often, normal buffered saline (135-150 mM NaCl) or 5% dextrose will be used. These compositions can be sterilized by conventional sterilization techniques, such as filtration. The resulting aqueous solutions may be packaged for use or filtered under aseptic conditions and lyophilized, the lyophilized preparation being combined with a sterile aqueous solution prior to administration. The compositions may also contain pharmaceutically acceptable auxiliary substances as required to approximate physiological conditions, such as pH adjusting and buffering agents, tonicity adjusting agents and the like, for example, sodium acetate, sodium lactate, sodium chloride, potassium chloride, calcium chloride, etc. Additionally, the composition may include lipid-protective agents, which protect lipids against free-radical and lipid-peroxidative damages on storage. Lipophilic free-radical quenchers, such as α-tocopherol and water-soluble iron-specific chelators, such as ferrioxamine, are suitable.

The amount of active agent administered per dose is selected to be above the minimal therapeutic dose but below a toxic dose. The choice of amount per dose will depend on a number of factors, such as the medical history of the patient, the use of other therapies, and the nature of the disease. In addition, the amount of active agent administered may be adjusted throughout treatment, depending on the patient's response to treatment and the presence or severity of any treatment-associated side effects. In certain embodiments, the dosage of LNP composition or the frequency of administration is approximately the same as the dosage and schedule of treatment with the corresponding free active agent. However, it is understood that the dosage may be higher or more frequently administered as compared to free drug treatment, particularly where the LNP composition exhibits reduced toxicity. It is also understood that the dosage may be lower or less frequently administered as compared to free drug treatment, particularly where the LNP composition exhibits increased efficacy as compared to the free drug. Exemplary dosages and treatment for a variety of chemotherapy compounds (free drug) are known and available to those skilled in the art and are described in, e.g., Physician's Cancer Chemotherapy Drug Manual, E. Chu and V. Devita (Jones and Bartlett, 2002).

Patients typically will receive at least two courses of such treatment, and potentially more, depending on the response of the patient to the treatment. In single agent regimens, total courses of treatment are determined by the patient and physician based on observed responses and toxicity.

### Combination Therapies

In certain embodiments, LNP compositions of the instant disclosure can be administered in combination with one or more additional compounds or therapies, such as surgery, radiation treatment, chemotherapy, or other active agents, including any of those described above. LNP compositions may be administered in combination with a second active agent for a variety of reasons, including increased efficacy or to reduce undesirable side effects. The LNP composition may be administered prior to, subsequent to, or simultaneously with the additional treatment. Furthermore, where a LNP composition of the instant disclosure (which comprises a first active agent) is administered in combination with a second active agent, the second active agent may be administered as a free drug, as an independent LNP formulation, or as a component of the LNP composition comprising the first drug. In certain embodiments, multiple active agents are loaded into the same lipid nanoparticles. In other embodiments, lipid nanoparticles comprising an active agent are used in combination with one or more free drugs. In particular embodiments, LNP compositions comprising an active agent are formed individually and subsequently combined with other compounds for a single co-administration. Alternatively, certain therapies are administered sequentially in a predetermined order. Accordingly, LNP compositions of the instant disclosure may comprise one or more active agents.

Other combination therapies known to those of skill in the art can be used in conjunction with the methods of the instant disclosure.

Unless otherwise defined, all technical and scientific terms used herein have the same meaning as commonly understood by one of ordinary skill in the art to which this disclosure belongs. Although methods and materials similar or equivalent to those described herein can be used in the practice or testing of the present disclosure, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Reference will now be made in detail to exemplary embodiments of the disclosure. While the disclosure will be described in conjunction with the exemplary embodiments, it will be understood that it is not intended to limit the disclosure to those embodiments. To the contrary, it is intended to cover alternatives, modifications, and equivalents as may be included within the spirit and scope of the disclosure as defined by the appended claims. Standard techniques well known in the art or the techniques specifically described below were utilized.

### EXAMPLES

### Example 1: Preparation of DOTAP-LNP Formulations of Different Parameters

DOTAP-based LNPs were prepared using a microfluidic mixing process. Briefly, lipid stocks of DOTAP, DOPC, CHE and PEG-DMG were prepared in ethanol at 20 mg/ml concentration. Different N/P ratios (2, 3, 4 or 6 as currently exemplified), PEGylation (0-2% as currently exemplified), and lipid compositions (molar ratio between the lipids to each other) were investigated. In all initial formulations, the DOTAP mol percent was kept at 45%, while later formulations included DOTAP at 25 mol %, 50 mol % and 75 mol % of total lipid in the particle. For the initial nucleic acid-particle formulations shown in Table 1 below, lipids were mixed together for the given compositions in ethanol with a final lipid concentration of 5.8 mg/ml. Firefly luciferase mRNA (mFluc) was used as the mRNA in the aqueous phase at a concentration of 0.25 mg/ml. The mixing of two phases and LNP preparation was performed using a 2:1 aqueous to organic volume ratio, and at an 8 ml/min flow rate in a microfluidic chip with staggered herringbone structure. Resulting LNPs were subjected to purification and buffer exchange by tangential flow filtration (TFF) against PBS. Table 1, below, summarizes the characterization parameters of the initially prepared formulations. Precise control of the characterization parameters enabled the preparation of DOTAP LNPs in the size range of 51-188 nm, with surface charges between 0-26 mV, and PDI values at or below 0.2. All formulations showed more than 98% of encapsulation efficiency (EE) calculated by Ribogreen assay using the manufacturer's protocol.

TABLE 1
DOTAP-Based Nucleic Acid-Lipid Particles and Characteristics

| | | | | | | | Zeta | | |
|---|---|---|---|---|---|---|---|---|---|
| | | | | | | | Potential | | |
| N/P | DOTAP | PEG | Size (nm) | | PDI | | (mV) | | EE |
| ratio | (mol %) | (mol %) | Mean | SD | Mean | SD | Mean | SD | (%) |
| 3 | 45 | 0 | 188 | 0 | 0.20 | 0.01 | 26 | 1 | 98.7 |
| 3 | 45 | 1 | 71 | 0 | 0.09 | 0.02 | 5 | 2 | 99.3 |
| 6 | 45 | 1 | 64 | 0 | 0.11 | 0.01 | 7 | 1 | 99.5 |
| 6 | 45 | 2 | 51 | 2 | 0.15 | 0.07 | 0 | 2 | 99.6 |

Notably, DOTAP sufficiently stabilized LNPs such that no polyethylene glycol (PEG)-conjugated lipids were required to achieve viable nanoparticles. Without wishing to be bound by theory, it is likely that DOTAP's high surface charge provided sufficient electrostatic stability to stabilize the particles. The ability as demonstrated herein to formulate PEG-free nucleic acid-lipid particles that are highly active for delivery of nucleic acid cargoes allows for mitigation or outright avoidance of previously observed disadvantages of PEGylated formulations, which include, without limitation, decreased cellular interaction and internalization, as well as PEG-driven Accelerated Blood Clearance (ABC), which is a well-documented phenomenon caused by the body's immune response to PEGylated lipids on the surface of LNPs. ABC is responsible for the clearance of nanoparticles from systemic circulation upon repeated dosing. Thus, the DOTAP-mediated stabilization of PEG-free LNPs discovered herein has provided an important therapeutic improvement in the delivery of LNP-associated nucleic acid cargoes.

### Example 2: DOTAP Lipid Nanoparticles (LNPs) Delivered Reporter mRNAs and Exhibited Low Toxicity

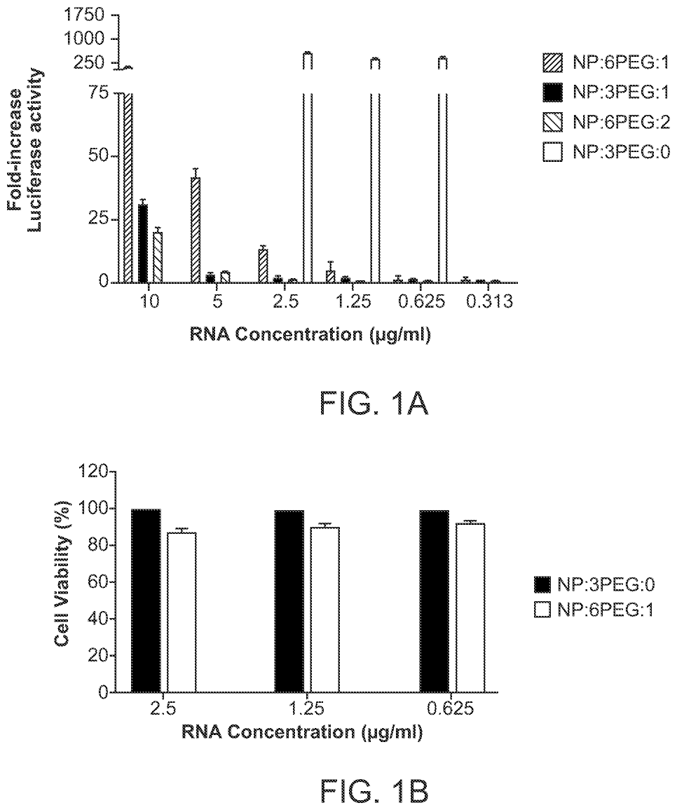

In an initial demonstration of delivery to cells and subsequent expression in cells of DOTAP LNP-associated mRNA reporters as cargo, DOTAP LNPs with mRNA reporter cargoes were tested in vitro on Hepa 1-6 cells (a murine cell line). Hepa 1-6 cells were first seeded in 96-well black-walled microplates with a plating density of 20,000 cells/well/100 μl. Cells were incubated at 37° C. under 5% CO₂ and allowed to attach overnight. The next day, cells were treated with DOTAP-LNP formulations as disclosed herein having mRNA concentrations varying from 0.313 μg/ml to 10 μg/ml, in complete medium for 24 hours continuously. FIG. 1A shows the luciferase enzyme activity observed for the tested LNP formulations. There was remarkably an approximately 600-fold increase in luciferase activity achieved with tested no-PEG formulations (0% PEG) as compared to tested PEG-containing formulations, and mFluc (luciferase) activity was observed to be dose-dependent as nucleic acid cargo concentrations increased (dose-dependence was particularly observed for PEG-containing formulations, whereas even low cargo concentration in PEG-free LNPs produced robust activity). High concentrations of the tested DOTAP-LNPs did not induce significant cytotoxicity, which demonstrated the low toxicity profile of the currently disclosed formulations (FIG. 1). These data demonstrated that the DOTAP-LNPs and associated cargoes of the instant disclosure were effectively internalized in vitro. DOTAP-LNP cargo activities were also retained intracellularly, with mRNA cargoes active in the cytoplasm of the cells, and low cytotoxicity was observed, indicative of the LNP formulations being safe for in vivo testing.

### Example 3: Tested DOTAP Lipid Nanoparticles (LNPs) Preferentially Delivered to Lung Tissue

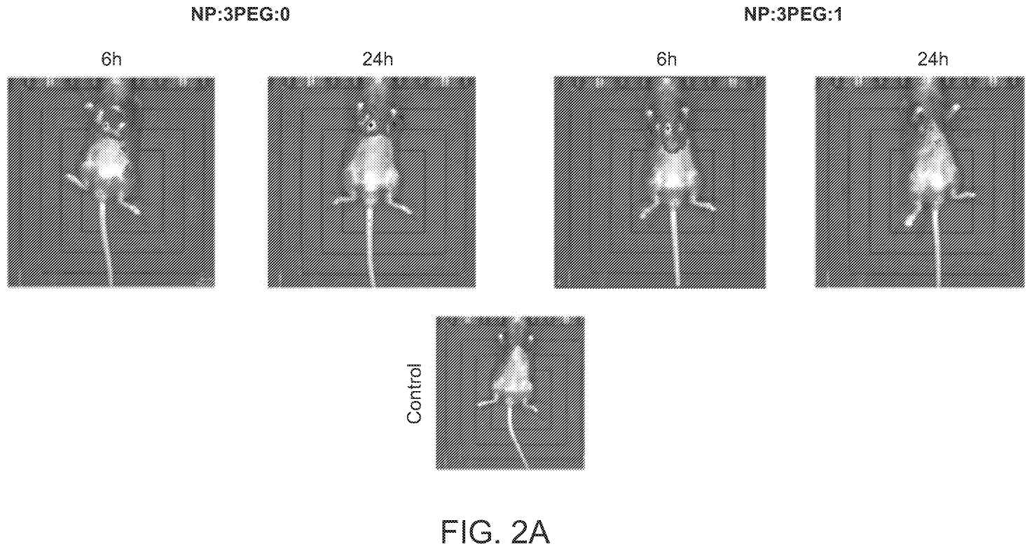

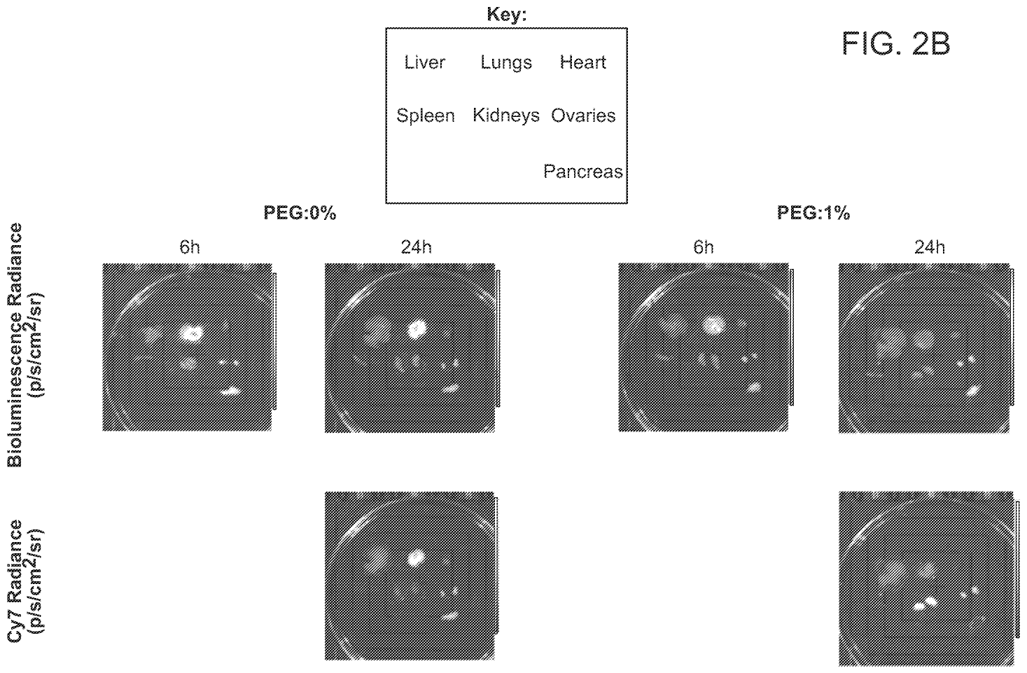

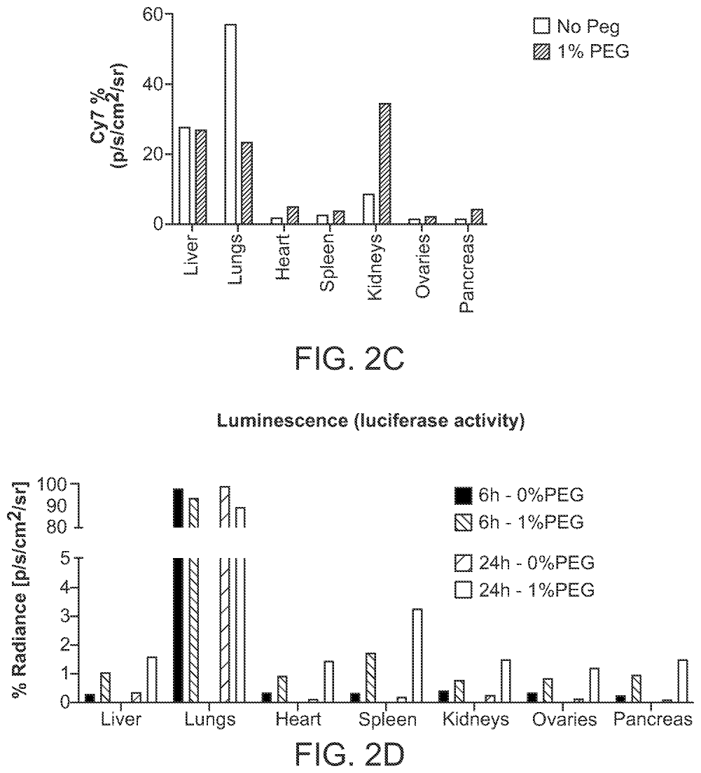

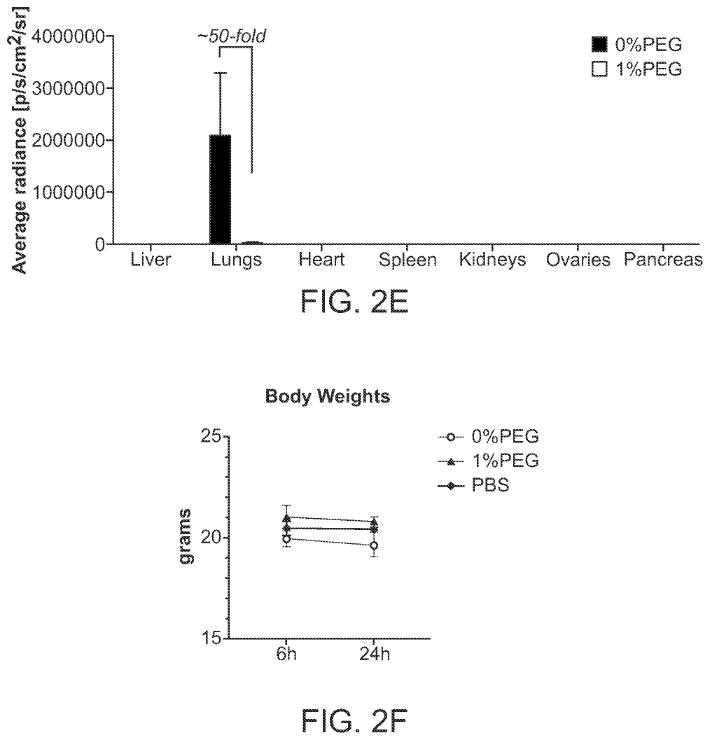

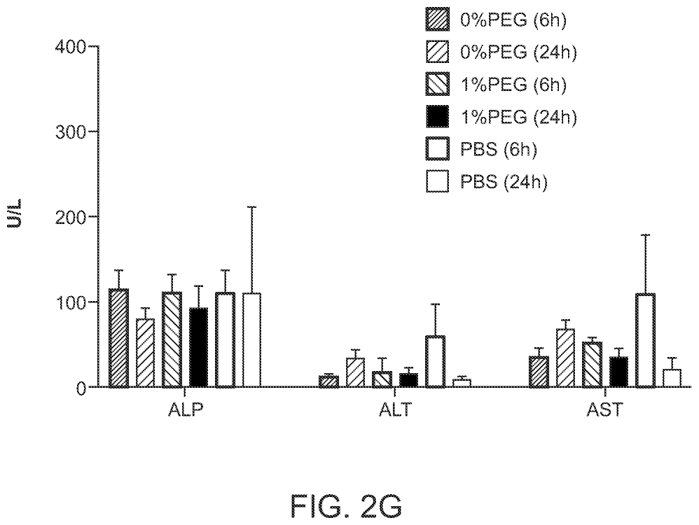

In vivo systemic, post-IV biodistribution of DOTAP-LNPs as disclosed herein harboring mRNA cargoes was assessed. DOTAP-LNPs possessing varying surface charges (0-26 mV) and PEGylation values (0-1%) were specifically examined in intravenously LNP-injected C57BL/6 mice via both in vivo imaging and ex vivo detection of delivery and cargo expression in harvested organs. DOTAP-LNP formulations possessing 45% (by mol) DOTAP were prepared as in Table 1 above, with (1%) and without (0%) PEG-DMG. LNPs were also fluorescently labeled with Cy7-DOPE in the formulation (0.5% mol). Briefly, mFluc mRNA-loaded DOTAP-LNPs were administered to mice at 3 mg/kg dose intravenously. At 6 hours and at 24 hours post-administration, 150 mg/kg luciferin in PBS was injected intraperitoneally, and mice were anesthetized under isoflurane for live animal fluorescence and luminescence imaging. Cy7 signal distribution indicated LNP biodistribution, while the luminescence signal indicated reporter mRNA cargo activity. Notably, both DOTAP-LNPs tested, N/P:3PEG:0 and N/P:3PEG: 1 (Table 1), demonstrated concentrated luciferase activity (and therefore both localization and expression) in mouse lungs (FIG. 2A).

Major organs of treated mice were then examined ex vivo. As shown in FIG. 2B, although tested DOTAP-LNPs were widely distributed to lung and other organs, DOTAP-LNP mRNA expression was highly specific to the lungs. Tested DOTAP-LNP delivery and expression of associated mRNA cargoes was observed in the lungs at levels exceeding 90% of all luminescence signal detected, despite wider LNP distribution having clearly occurred (FIG. 2D). Another surprising result was that DOTAP-LNPs having 0% PEG exhibited approximately 50-fold higher mRNA expression (as evidenced by luciferase activity) in the lungs than did DOTAP-LNPs having 1% PEG (FIG. 2E). These data demonstrated that the lung selectivity observed herein for tested DOTAP-LNP mRNA delivery and expression was not due to LNP surface charge alone, but also without wishing to be bound by theory, was likely caused by an apparent structural affinity between DOTAP and the lung epithelium. Body weight and liver function tests also indicated that the DOTAP-LNPs of the instant disclosure were not toxic in vivo within 24 hours post-IV administration (FIGS. 2F and 2G).

### Example 4: Preferential Delivery of Tested DOTAP-LNPs to Lung Tissue was Unaffected by PEG-Lipid Chemistries

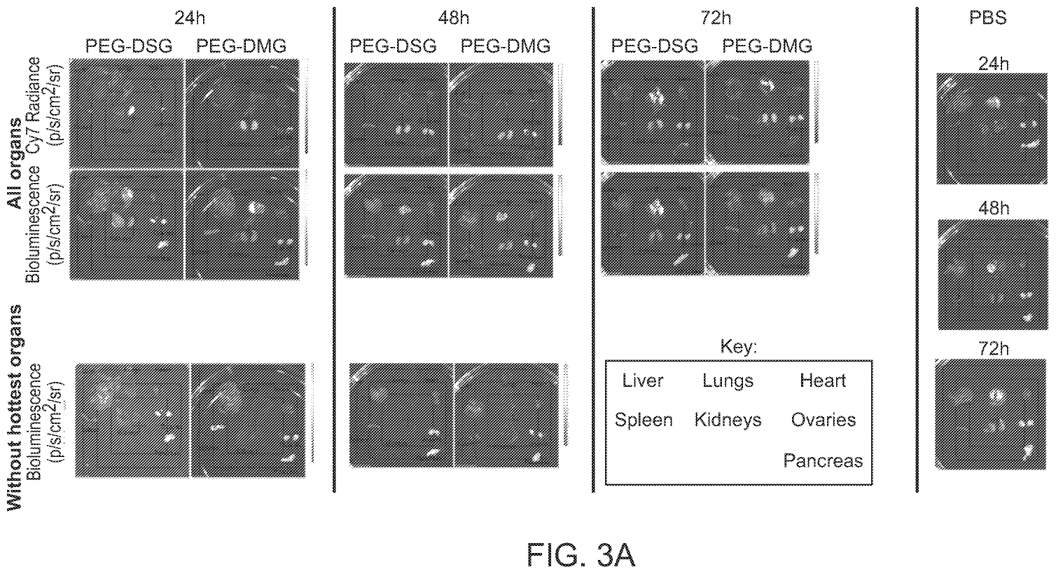

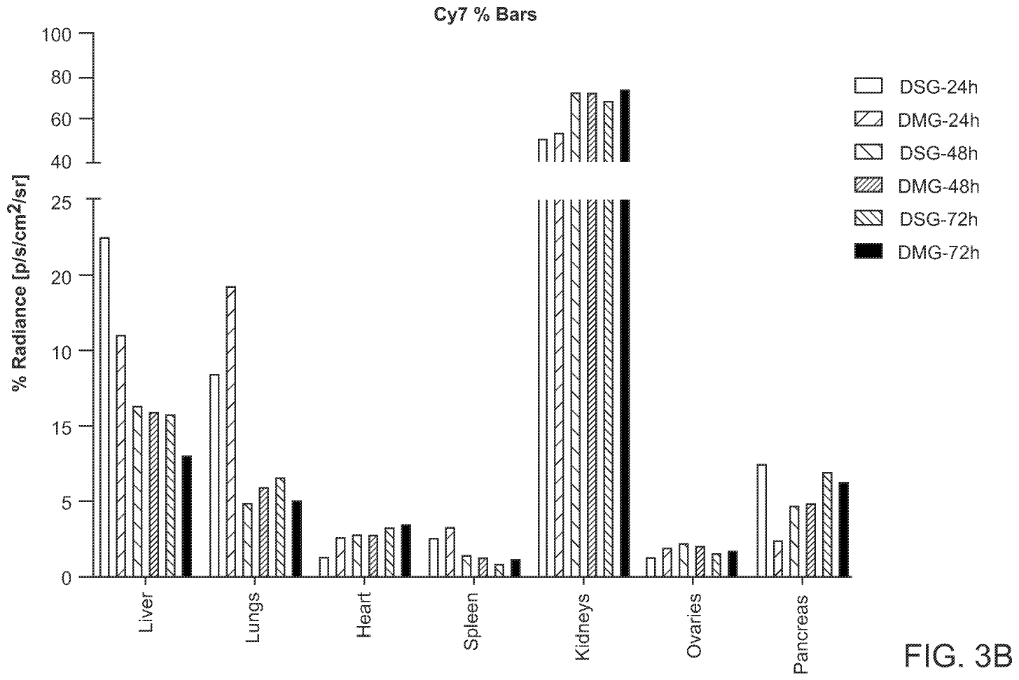

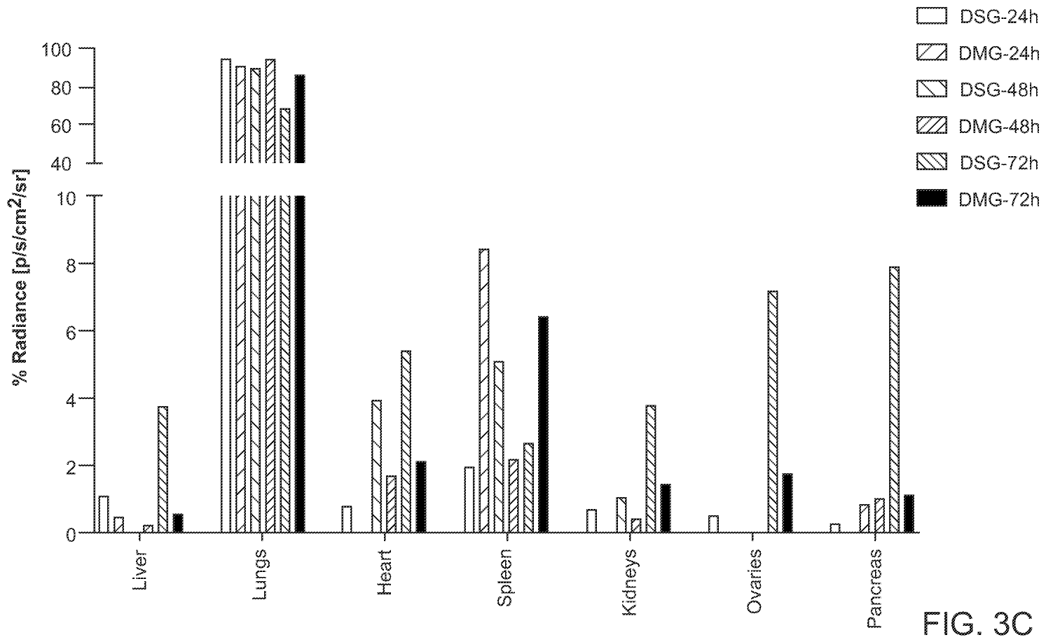

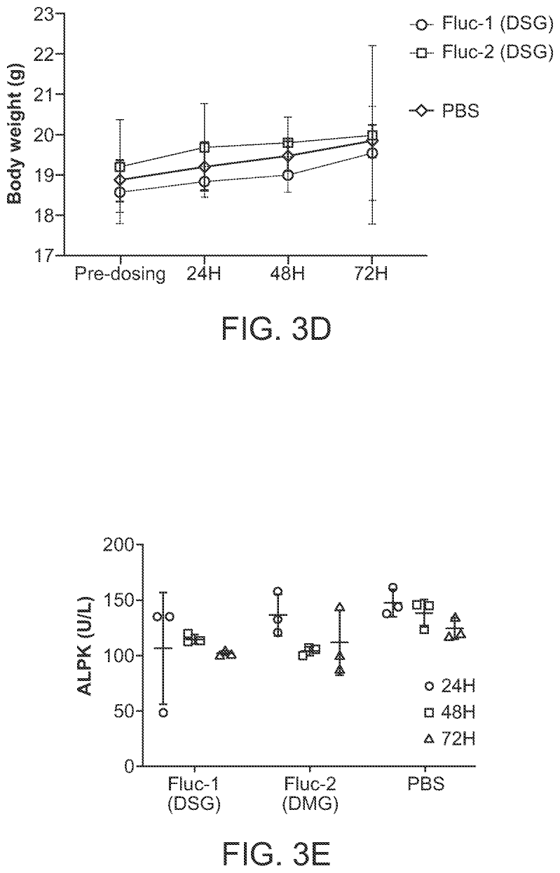

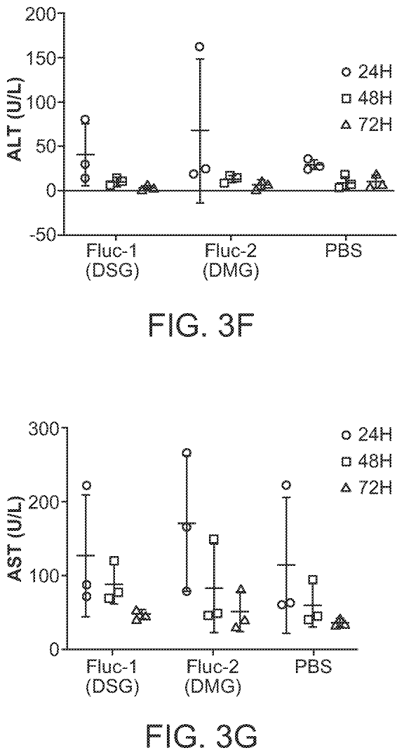

The greater mRNA activity observed for DOTAP-LNPs containing no PEGylated lipids compared to DOTAP-LNPs that contain 1% PEGylated lipids prompted further investigation into whether the identity of the lipid to which PEG is attached influences cargo delivery by DOTAP-LNPs. Such experiments demonstrated the type of lipid conjugated to PEG in tested formulations appeared to have no effect on organ targeting. Briefly, C57BL/6 mice were treated intravenously with DOTAP-LNPs containing 1 mol % of either PEG-DSG or PEG-DMG at 3 mg/kg dose. mFluc was used as the reporter mRNA cargo present in the LNPs, and the luminescence signal was measured upon administration of luciferin. DOTAP in the formulations was maintained at 45 mol %. Independent of the PEG-lipid type, mRNA-based luciferase activity was observed as contained in the lungs at 24, 48, and 72 hour timepoints. Both PEG-DSG and PEG-DMG formulations showed wide distribution of the LNPs; however, kidneys were the major organ of observed LNP accumulation (FIGS. 3A-3C). These results identified the kidneys as the likely excretion route for the tested LNPs. Lungs were also removed from initial imaging, and the remaining organs were re-imaged to obtain a higher signal-to-background ratio of luciferase radiance, as a high signal from a particular organ (here, the lungs) can mask lower but still significant signals emanating from other organs. The luciferase signal in the remaining organs was negligible compared to that of the lungs (FIG. 3C). Further, both PEG-DSG and PEG-DMG formulations did not show any signs of toxicity in mice, as they did not induce significant changes in body weight or liver function values (FIGS. 3D and 3E).

### Example 5: Tested DOTAP-LNPs Delivered Active Cre mRNA Reporter System as Cargo to Lung Cell Nuclei

Having identified that the DOTAP-LNPs disclosed herein exhibited preferential lung targeting to cellular cytoplasm for exemplified mRNA cargoes, which was independent of LNP particle size, LNP surface charge and PEGylation type used, it was then assessed whether such LNPs could also achieve nuclear delivery of a Cre mRNA reporter system in target lung tissue (e.g., as indication of whether nuclear delivery of other systems, such as CRISPR/Cas, nucleic acid modulating controllers, etc., could also be achieved using DOTAP-LNPs as described herein). Specifically, the mFluc reporter system of the above Examples required mRNA delivery into the cytoplasm of transduced cells of organs and tissues. However, nucleic acid modulating controllers, and other embodiments of therapeutic mRNAs, require translocation of the expressed protein into the nucleus to regulate a target gene of interest. The Cre recombinase enzyme system used as a test cargo for nuclear delivery also requires translocation of an mRNA-encoded protein to the nucleus and thus can serve as a reporter for effective nuclear translocation. When delivered to the nuclease of a cell having loxP sites (e.g., “floxed” for “flanked by loxP” target genes), Cre recombinase catalyzes site-specific recombination of DNA between loxP sites.

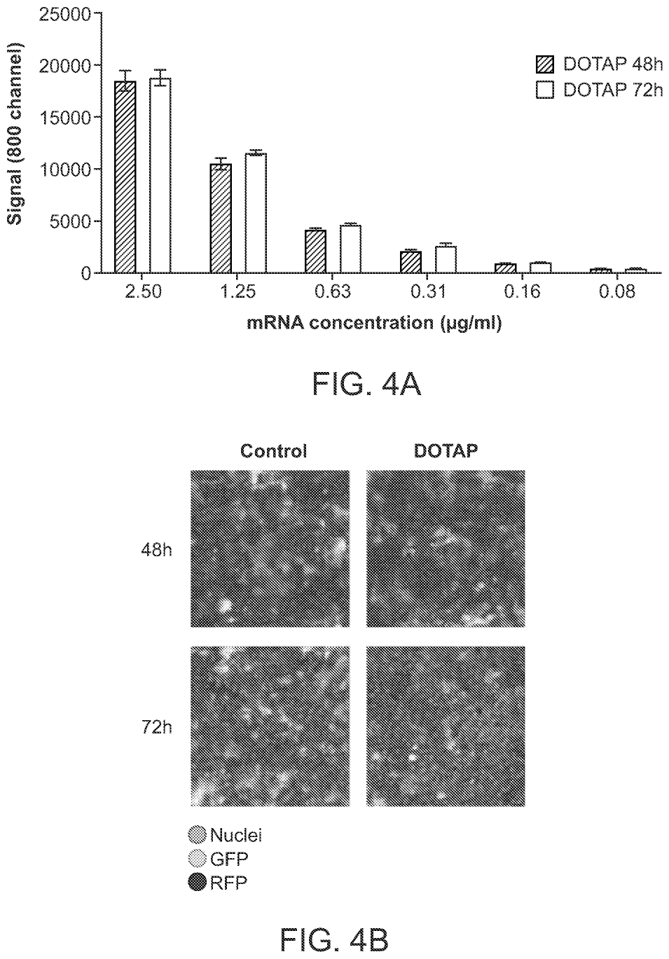

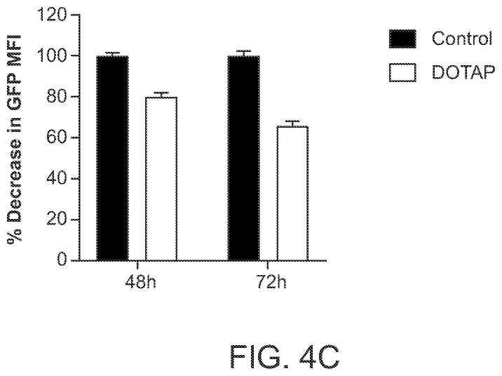

DOTAP-LNPs were again prepared having 45 mol % DOTAP and 1 mol % PEG-DMG encapsulating Cre recombinase mRNA (mCre) as cargo. Particle size, PDI and zeta potential of the mCre-loaded DOTAP-LNPs were measured as 58.6±0.6 nm, 0.09±0.03 and 1.2±0.6 mV, respectively. More than 98% of the mCre was associated in the formulated DOTAP-LNPs. Cellular association of the DOTAP-LNPs disclosed herein was not affected by different mRNAs. FIG. 4A shows dose-dependent cellular association of the Cre-carrying DOTAP-LNPs disclosed herein with the HEK293-loxP-GFP-RFP cell line. This cell line stably expressed GFP signal, yet upon Cre recombinase expression and activity in the nucleus of target cells, due to Cre-mediated loxP recombination, the cells started to express RFP instead of GFP (FIG. 4B). mCre delivery and activity was also confirmed via use of flow cytometry, which measured disappearance of GFP signal in cells to which Cre successfully delivered and was expressed (FIG. 4C).

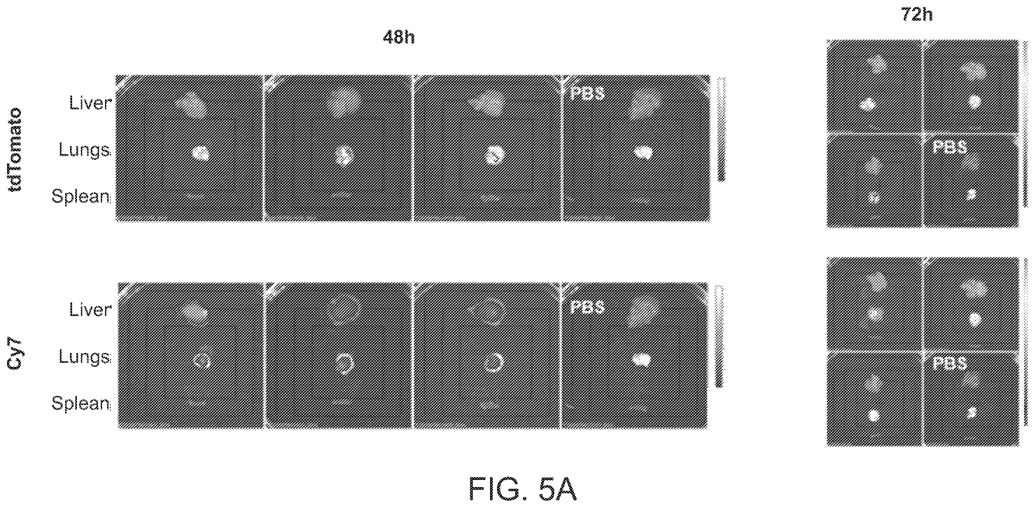

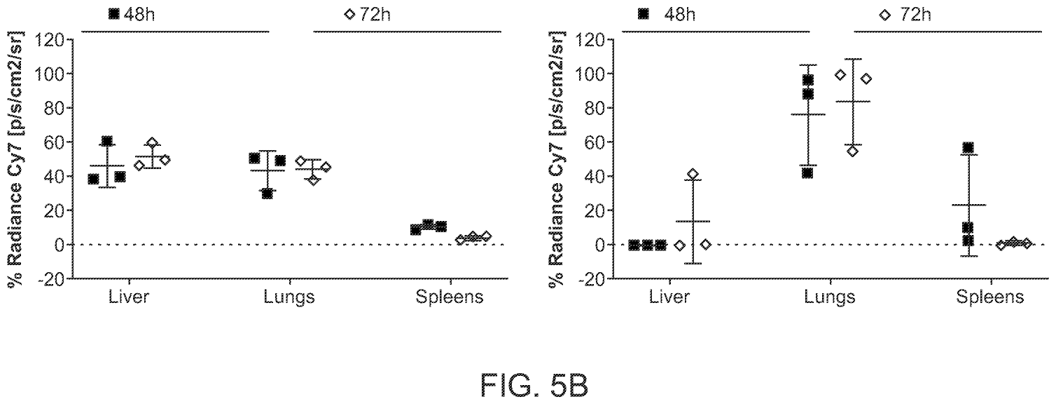

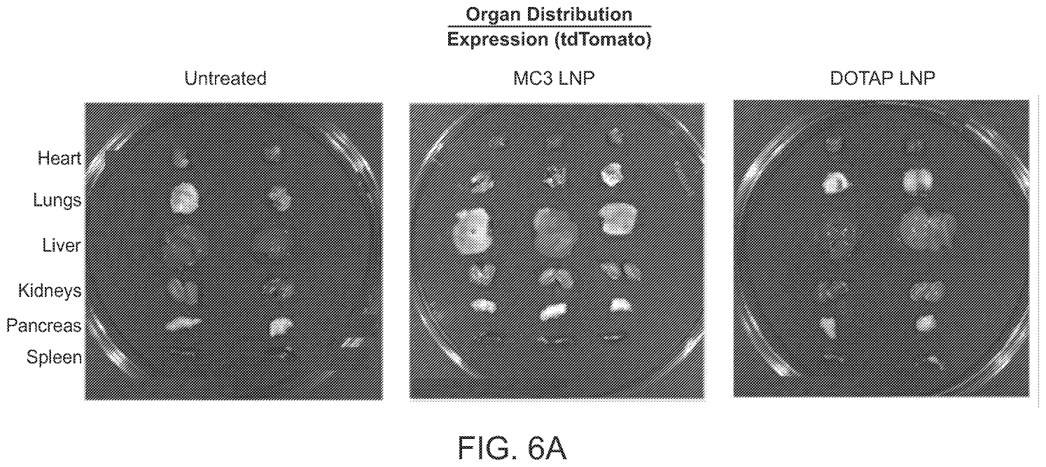

DOTAP-LNPs as disclosed herein also delivered nucleic acid cargoes that targeted the genome of lung cells in vivo. The biodistribution of mCre-loaded DOTAP-LNPs as disclosed herein in Ail4 mice (B6.Cg-Gt(ROSA)26Sor^{tm14(CAG-tdTmato)Hze/}J) was examined. Ail4 is a Cre reporter strain designed to have a loxP-flanked (“floxed”) STOP cassette that prevents transcription of a CAG promoter-driven red fluorescent protein variant (tdTomato)—all inserted into the Gt(ROSA)26Sor locus. Ai14 mice expressed robust tdTomato fluorescence following Cre-mediated recombination. Briefly, the Ail4 mice were intravenously injected with Cy7-labeled mCre-loaded DOTAP-LNPs at 3 mg/kg doses. 48 hours and 72 hours after LNP dosing, selected organs from the mice were collected, and ex vivo organ imaging was performed to evaluate both LNP distribution and organ-specific activity. Ex vivo imaging at 48 hours and 72 hours after dosing showed that the tested DOTAP-LNPs exhibited lung-specific activity regardless of particle distributions observed for individual types of DOTAP-LNPs (FIG. 5A). These results demonstrated that DOTAP-LNPs as disclosed herein delivered mRNA cargoes in a manner that promoted the expression and activity of proteins encoded by such mRNA cargoes in the nucleus in vivo. Moreover, despite the almost neutral charge and wide distribution of tested DOTAP-LNPs within different organs, cargo mRNA activity was confined to the lungs, emphasizing the structural affinity of DOTAP for lung tissues for efficient cytoplasmic mRNA delivery (and ultimate nuclear activity of proteins encoded by cargo mRNAs directed to nuclear targets). As shown in FIG. 5B, despite an approximately equal distribution of tested DOTAP-LNPs between liver and lungs, mRNA cargo-encoded Cre enzyme activity was only observed in the lungs—with the exception of one animal outlier. These results demonstrated that DOTAP-LNPs as disclosed herein can deliver nuclear-directed nucleic acid modulating controllers (i.e., mRNAs encoding for protein controller components, e.g., Zinc-Finger protein (ZFP) or other DNA- or RNA-binding domains associated with epigenetic regulators and/or nucleases) and other nucleic acid cargoes that show their effect on the genome, specifically in the lung.

### Example 6: Tested DOTAP-LNPs Transduced all Examined Lung Cell Types

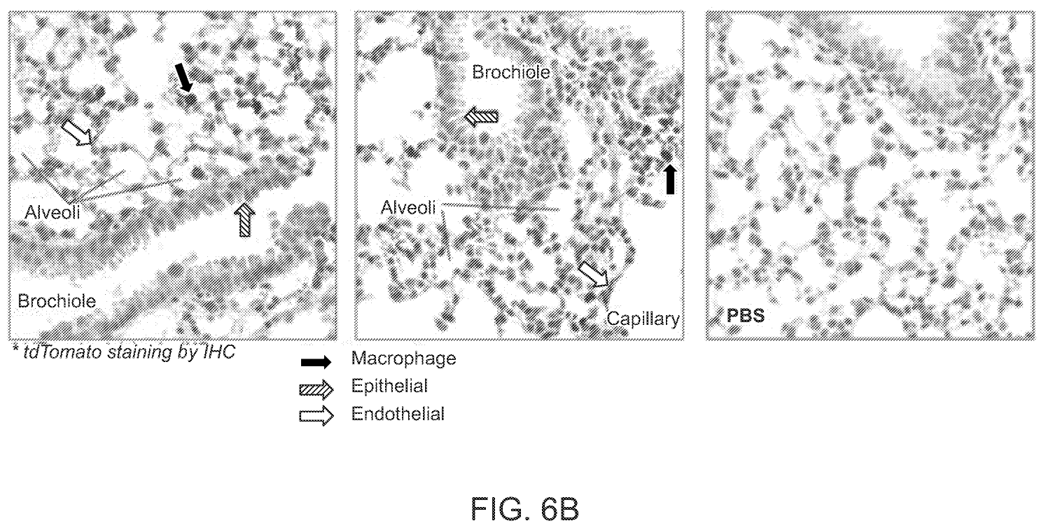

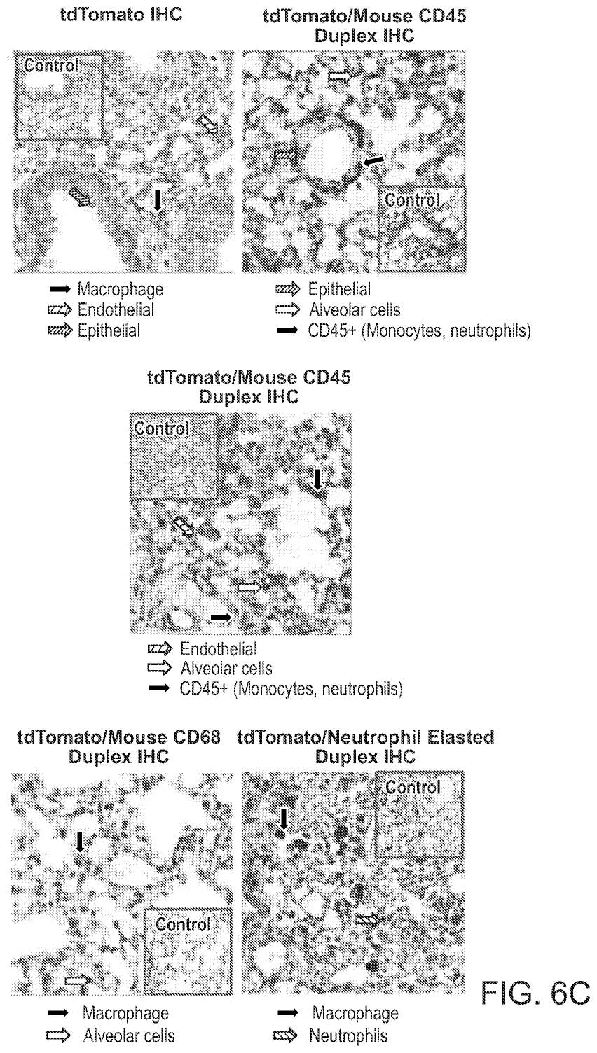

To determine whether DOTAP-LNPs as disclosed herein transduced all cell types in the lung, tdTomato signal in the liver, lungs and spleen harvested from an LNP-treated Ail4 mouse was evaluated. Lungs from the Ail4 animals treated intravenously with mCre-loaded DOTAP-LNPs having 1% PEG-DMG were evaluated using immunohistochemistry methods (IHC) to measure tdTomato expression levels in different cell populations of both healthy (wild-type) mice and mice having inflamed lungs (NSG-SGM3 mouse) (FIGS. 6A-6C). Macrophage, epithelial, and endothelial cells were all visibly transduced in all types of mice examined, which demonstrated that DOTAP-LNPs as disclosed herein transduced both progenitor and epithelial cells after IV administration. These results demonstrated that the DOTAP-LNPs disclosed herein promoted lung-specific delivery of nucleic acid cargoes and indicated that the use of such DOTAP-LNPs to deliver nucleic acid modulating cargoes may provide successful therapies for treatment of lung diseases and disorders involving all manner of lung tissue types, including in inflamed lungs.

### Example 7: DOTAP-LNP Characteristics Varied Across Tested Formulation Parameters, Resulting in Identification of LNP Formulations Possessing Improved Activity

To understand the edge of failure around the formulation design parameters and also to optimize DOTAP-LNP delivery, a statistical design of experiments (DoE)-based formulation development process was performed. The factors and their levels were defined as DOTAP (by mol %, 25-75), PEG (by mol %, 0-1.5) and N/P ratio (here, 2-4). Table 2, below, summarizes the formulations of the current Example, together with composition and characterization results for each. Preliminary LNP formulation process success criteria were defined as <200 nm particle size and <0.25 PDI. As can be seen from the table, two of the formulations having 50 mol % DOTAP (DOE-1 and DOE-3) failed these preliminary success criteria. These results indicated that the particle size and surface charge of the DOTAP-LNP could be controlled via manipulation of formulation parameters.

TABLE 2
Nucleic Acid-Lipid Particle Formulations

| | | | | | | | Lipid/ | | | | | | | |
|---|---|---|---|---|---|---|---|---|---|---|---|---|---|---|
| | | | | | | | RNA | Lipid | Particle | | | | | |
| | PEG | % | % DOPC | % | % | N/P | mass | mass | size (nm) | | PDI | | Zeta(mV) | |
| LNPs | type | PEG | (helper) | Chol. | DOTAP | ratio | ratio | (mg) | Ave. | SD | Ave. | SD | Ave. | SD |
| DOE-1 | PEG2k- | 1.5 | 10 | 38.25 | 50 | 4 | 15 | 7.6 | 72.0 | 0.3 | 0.33* | 0.004 | 4.5 | 2.6 |
| DOE-2 | DMG | 1 | 10 | 38.75 | 50 | 3 | 11 | 5.6 | 55.3 | 0.3 | 0.127 | 0.007 | 2.7 | 2.2 |
| DOE-3 | | 0 | 10 | 39.75 | 50 | 2 | 7 | 3.6 | 849* | 58.4 | 0.372 | 0.088 | 21.7 | 0.7 |
| DOE-4 | | 0 | 10 | 14.75 | 75 | 4 | 11 | 5.4 | 111.0 | 0.6 | 0.153 | 0.011 | 20.5 | 2.1 |
| DOE-5 | | 1 | 10 | 63.75 | 25 | 4 | 26 | 13.0 | 68.0 | 1.4 | 0.133 | 0.035 | 1.7 | 2.6 |
| DOE-6 | | 1.5 | 10 | 63.25 | 25 | 2 | 13 | 6.6 | 58.8 | 1.6 | 0.133 | 0.077 | −1.0 | 0.5 |
| DOE-7 | | 0 | 10 | 64.75 | 25 | 3 | 19 | 9.4 | 184.9 | 3.1 | 0.158 | 0.030 | 17.3 | 2.0 |
| DOE-8 | | 0.5 | 10 | 39.25 | 50 | 3 | 11 | 5.5 | 83.5 | 0.4 | 0.203 | 0.021 | 7.5 | 1.9 |
| DOE-9 | | 1.5 | 10 | 13.25 | 75 | 3 | 9 | 4.3 | 48.8 | 0.7 | 0.153 | 0.023 | 3.5 | 1.0 |
| DOE-10 | | 1 | 10 | 13.75 | 75 | 2 | 6 | 2.8 | 94.4 | 2.7 | 0.151 | 0.084 | 3.0 | 1.3 |
| DOE-11 | | 0.5 | 10 | 14.25 | 75 | 2 | 6 | 2.8 | 157.3 | 2.7 | 0.175 | 0.063 | 7.7 | 1.4 |
| DOE-12 | | 0.5 | 10 | 64.25 | 25 | 4 | 25 | 12.7 | 72.8 | 1.1 | 0.146 | 0.050 | 5.2 | 1.0 |
| DOE-13 | | 0 | 10 | 39.75 | 50 | 3 | 11 | 5.4 | 124.9 | 0.8 | 0.142 | 0.009 | 26.2 | 2.7 |
| DOE-14 | | 1 | 10 | 38.75 | 50 | 2 | 7 | 3.7 | 63.9 | 4.5 | 0.246 | 0.189 | 1.3 | 1.9 |

*Indicates DOE was not selected due to indicated formulation parameter.

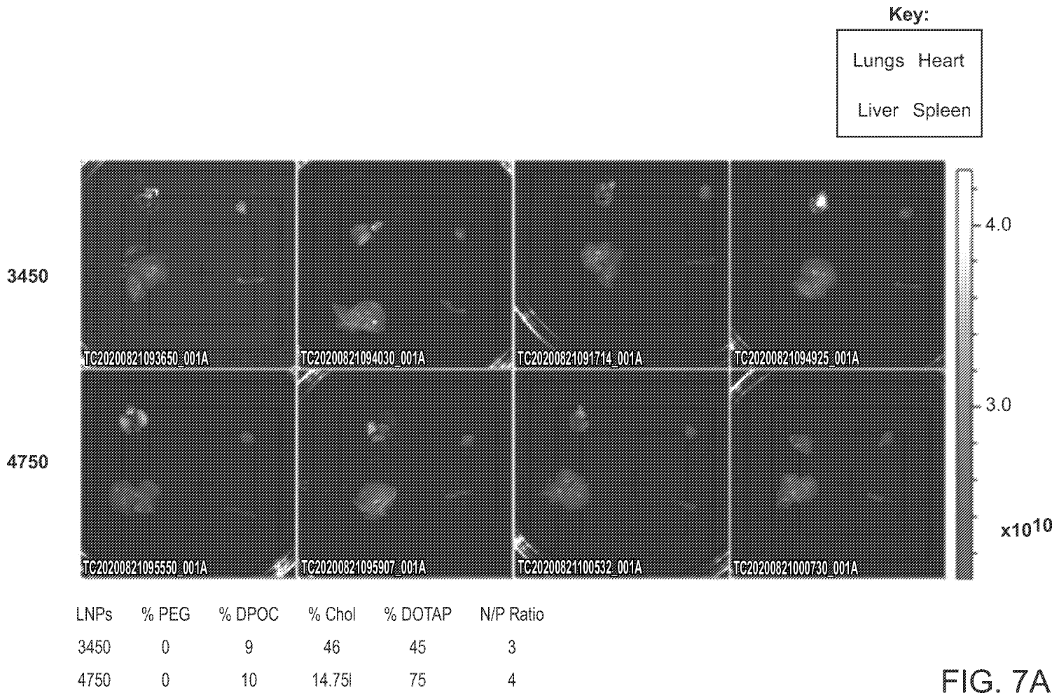

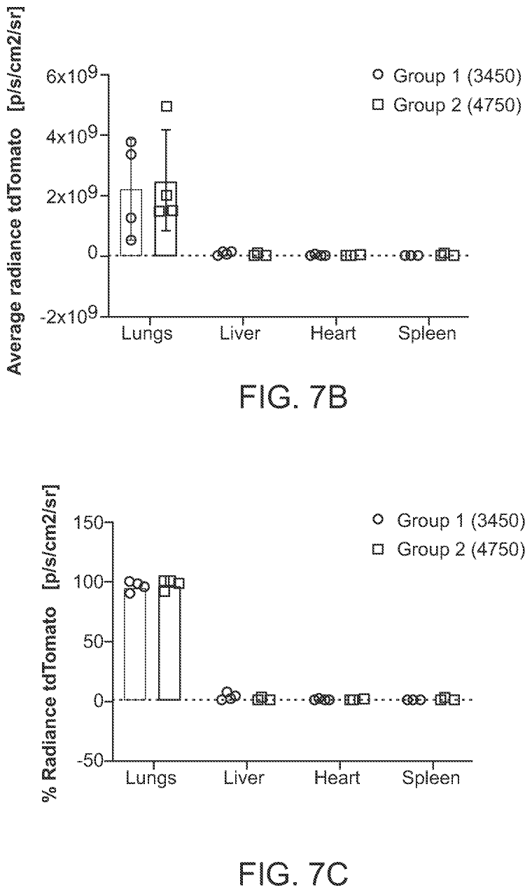

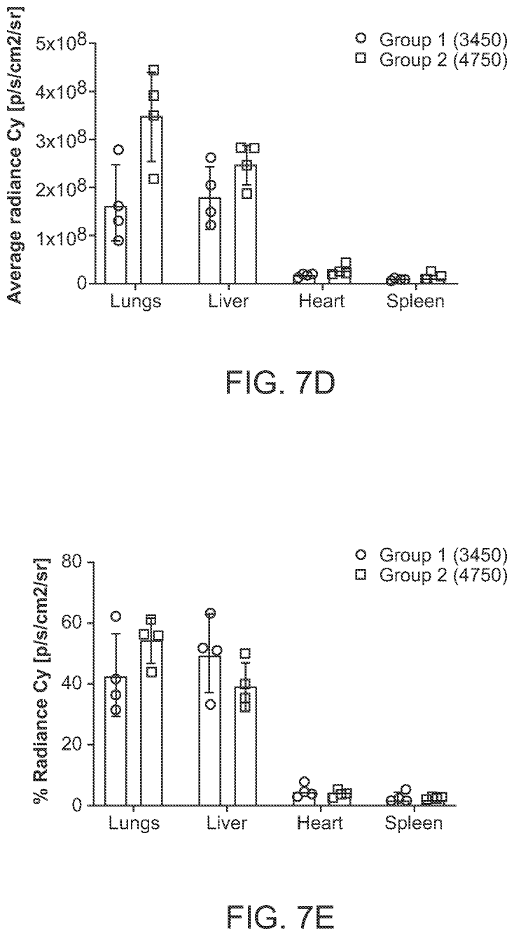

Based on having performed the formulation optimization with DoE, two formulations were selected for further assessment of their biodistributions in the Ail4 mice model as previously described. Formulation “3450” (DOTAP-LNP with 0% PEG and 45% DOTAP) and formulation “4750” (optimized DOTAP-LNP with 0% PEG and 75% DOTAP) were each loaded with mCre mRNA cargo and injected intravenously into Ail4 mice at a 3 mg/kg dosage. Both formulations contained the same amounts of Cy7, and the total lipid concentrations were the same. Both the 3450 and 4750 formulations exhibited mRNA activity only in the lungs (FIG. 7A). Further, the average tdTomato signal levels in the lungs, liver, heart, and spleen were not significantly different between the two formulations tested (FIG. 7B). For both formulations, nearly 100% of observed tdTomato expression was in the lungs.

The tdTomato signal production was not dose-dependent and can be described as always on-or-off. However, the 4750 formulation demonstrably delivered higher amounts of LNPs (FIGS. 7B-7E), as shown by imaging of the Cy7 fluorescently labeled lipid, than did the 3450 formulation. Therefore, the 4750 formulation resulted in higher LNP delivery in the lung tissue and may be selected for treatments where dose-dependent nucleic acid cargo delivery is required.

As shown in Table 3, the characterization parameters of DOTAP-LNP formulations were maintained with associated nucleic acid modulating controllers. Assessed formulations exhibited 60-380 nm particle size, neutral-to-positive surface charge, and favorable PDI values.

TABLE 3
Therapeutic Nucleic Acid Cargo-Loaded LNPs and Characteristics

| | | | | mRNA | | | | |
|---|---|---|---|---|---|---|---|---|
| PEG | | | | length | Size | Zeta | | EE |
| (%) | Formulation | mRNA | ID | (bases) | (nm) | (mV) | PDI | (%) |
| 0 | 4750 | dCas9-EZH2 | MR-28938-2 | 6775 | 386 ± 6 | 17 ± 2 | 0.314 | All |
| 1 | 3451 | dCas9-EZH2 | MR-28938-2 | 6775 | 85 ± 1 | 5 ± 1 | 0.104 | formulation |
| 1 | 3451 | dCas9-MQ1 | MR-28125-3 | 5705 | 69 ± 0 | 2 ± 2 | 0.104 | have very |
| 0 | 4750 | ZF AGA6-CDK9 | MR-29485-3 | 2431 | 180 ± 9 | 31 ± 1 | 0.084 | high |
| 0.5 | 47505 | ZF AGA6-CDK9 | MR-29485-3 | 2431 | 109 ± 1 | 16 ± 1 | 0.370 | encapsulation |
| 1 | 4751 | ZF AGA6-CDK9 | MR-29485-3 | 2431 | 86 ± 1 | 7 ± 2 | 0.209 | efficiency, |
| 0.5 | 47505 | dCas9-EZH2 | MR-28938-2 | 6775 | 122 ± 0 | 14 ± 1 | 0.134 | average of |
| 0 | 6750 | dCas9-EZH2 | MR-28938-2 | 6775 | 157 ± 0 | 31 ± 1 | 0.184 | 96% |

### Example 8: DOTAP-LNPs Also Successfully Delivered Nucleic Acid Cargoes Via Direct Injection/Localized Administration

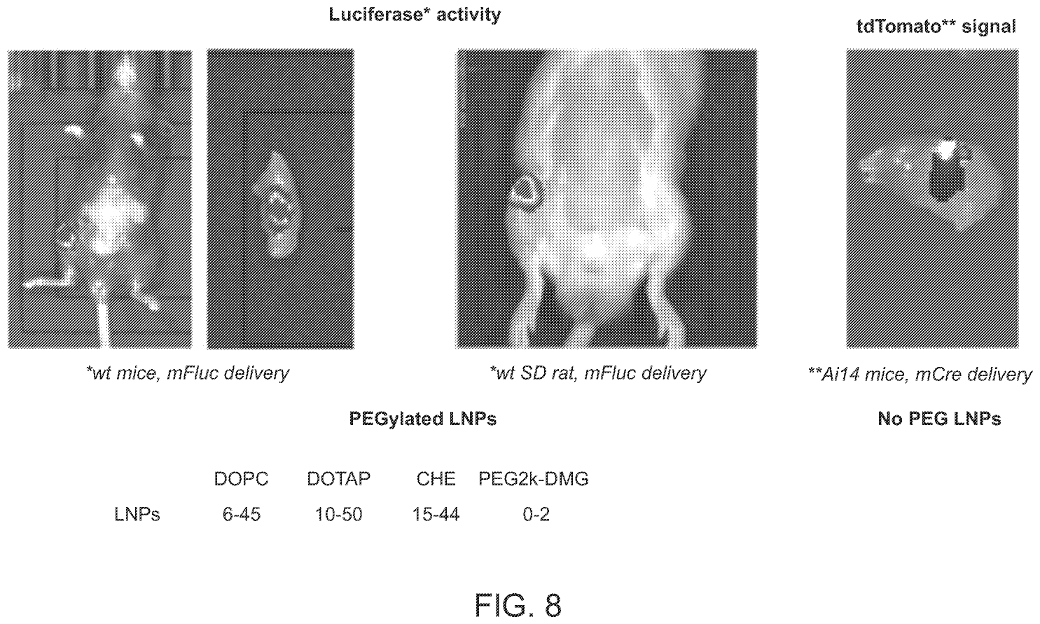

The remarkably lung cell-directed delivery of active nucleic acid cargoes via IV administration of DOTAP-LNPs as disclosed herein is described above. To test whether DOTAP-LNP-mediated delivery of mRNA cargoes could occur via direct injection to local tissues, DOTAP-LNPs with associated mRNA reporters were injected (intra-articular) into the knees of mice and rats. FIG. 8 shows the successful integration of the above-described reporter systems mFluc and mCre by tested DOTAP-LNPs into the local tissue area, thereby demonstrating both cytoplasmic and nuclear activities of cargoes in targeted tissue cells.

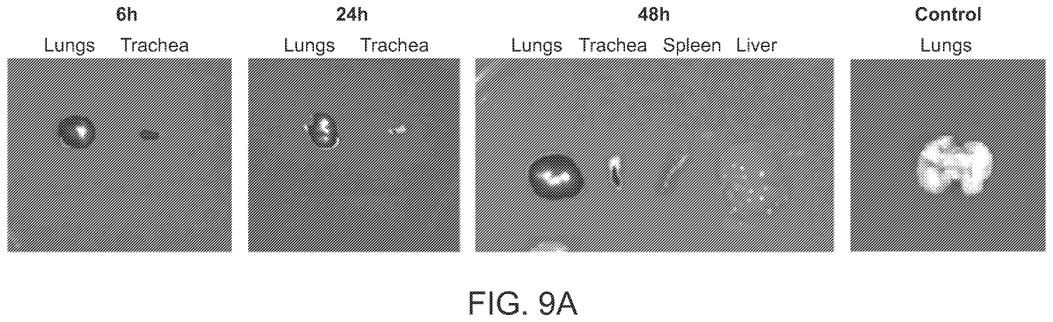

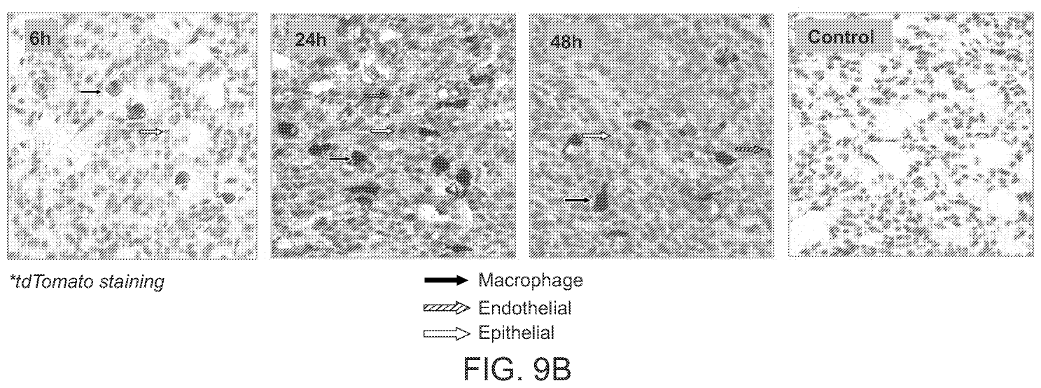

Intratracheal administration of DOTAP-LNPs harboring associated mRNA reporter cargoes also resulted in successful delivery of nucleic acid cargoes to the lung tissue. Specifically, local delivery of tested DOTAP-LNPs to lungs using Ail4 mice and intratracheal (topical) instillation was observed (FIGS. 9A and 9B). DOTAP-LNPs with 0% PEG were administered locally to Ail4 mice at 15 μg mCre/animal. Time-dependent imaging at 6, 24, and 48 hours showed that local administration of tested mCre-loaded DOTAP-LNPs started to show their effect in the lungs and trachea as early as 6 hours post-administration (FIG. 9A). No off-target effects were observed in spleen or liver (FIG. 9A). Immunohistochemistry staining of lung tissue sections further exhibited tested mCre mRNA cargo-loaded DOTAP-LNPs to have accessed key cell types (including macrophages, endothelial and epithelial cells) via intratracheal (topical) instillation, even as early as at 6 h post-administration (FIG. 9B). These results demonstrated that DOTAP-LNP delivery of nucleic acid cargoes as disclosed herein was effective and likely appropriate for local administration into the lung in clinical cases that require airway-associated cell activity.

All patents and publications mentioned in the specification are indicative of the levels of skill of those skilled in the art to which the disclosure pertains. All references cited in this disclosure are incorporated by reference to the same extent as if each reference had been incorporated by reference in its entirety individually.

One skilled in the art would readily appreciate that the present disclosure is well adapted to carry out the objects and obtain the ends and advantages mentioned, as well as those inherent therein. The methods and compositions described herein as presently representative of preferred embodiments are exemplary and are not intended as limitations on the scope of the disclosure. Changes therein and other uses will occur to those skilled in the art, which are encompassed within the spirit of the disclosure, are defined by the scope of the claims.

In addition, where features or aspects of the disclosure are described in terms of Markush groups or other grouping of alternatives, those skilled in the art will recognize that the disclosure is also thereby described in terms of any individual member or subgroup of members of the Markush group or other group.

The use of the terms “a” and “an” and “the” and similar referents in the context of describing the disclosure (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein.

All methods described herein can be performed in any suitable order unless otherwise indicated herein or otherwise clearly contradicted by context. The use of any and all examples, or exemplary language (e.g., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure.

Embodiments of this disclosure are described herein, including the best mode known to the inventors for carrying out the disclosed invention. Variations of those embodiments may become apparent to those of ordinary skill in the art upon reading the foregoing description.

The disclosure illustratively described herein suitably can be practiced in the absence of any element or elements, limitation or limitations that are not specifically disclosed herein. Thus, for example, in each instance herein any of the terms “comprising”, “consisting essentially of”, and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present disclosure provides preferred embodiments, optional features, modification and variation of the concepts herein disclosed may be resorted to by those skilled in the art, and that such modifications and variations are considered to be within the scope of this disclosure as defined by the description and the appended claims.

It will be readily apparent to one skilled in the art that varying substitutions and modifications can be made to the invention disclosed herein without departing from the scope and spirit of the invention. Thus, such additional embodiments are within the scope of the present disclosure and the following claims. The present disclosure teaches one skilled in the art to test various combinations and/or substitutions of chemical modifications described herein toward generating conjugates possessing improved contrast, diagnostic and/or imaging activity. Therefore, the specific embodiments described herein are not limiting and one skilled in the art can readily appreciate that specific combinations of the modifications described herein can be tested without undue experimentation toward identifying conjugates possessing improved contrast, diagnostic and/or imaging activity.

The inventors expect skilled artisans to employ such variations as appropriate, and the inventors intend for the disclosure to be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context. Those skilled in the art will recognize, or be able to ascertain using no more than routine experimentation, many equivalents to the specific embodiments of the disclosure described herein. Such equivalents are intended to be encompassed by the following claims.

1. A nucleic acid-lipid particle for delivering a nucleic acid cargo to a lung tissue of a subject, the nucleic acid-lipid particle comprising 1,2-dioleoyl-3-trimethylammonium-propane (DOTAP) comprising from 20 mol % to 80 mol % of the total lipid present in the nucleic acid-lipid particle.

2. The nucleic acid-lipid particle of claim 1 comprising a conjugated lipid that inhibits aggregation of particles comprising from 0.01 to 2% of the total lipid present, optionally wherein the conjugated lipid comprises a polyethyleneglycol (PEG)-lipid conjugate, optionally wherein the PEG of the PEG-lipid conjugate has an average molecular weight of from 550 daltons to 3000 daltons, optionally wherein the PEG-lipid conjugate is a PEG2000-lipid conjugate, optionally wherein the PEG2000-lipid conjugate comprises one or more of 1,2-dimyristoyl-rac-glycero-3-methoxypolyethylene glycol-2000 (DMG-PEG2k) and 1,2-distearoyl-rac-glycero-3-methoxypolyethylene glycol-2000 (DSG-PEG2k), optionally wherein the PEG2000-lipid conjugate is 1,2-Dimyristoyl-rac-glycero-3-methoxypolyethylene glycol-2000 (DMG-PEG2k), optionally wherein the nucleic acid-lipid particle comprises a PEG-lipid conjugate at a concentration selected from the group consisting of about 0.5 mol % of the total lipid present in the nucleic acid-lipid particle, about 1.0 mol % of the total lipid present in the nucleic acid-lipid particle, and about 1.5 mol % of the total lipid present in the nucleic acid-lipid particle.

3. The nucleic acid-lipid particle of claim 1, wherein the nucleic acid-lipid particle does not comprise a PEG-lipid conjugate, optionally wherein the nucleic acid-lipid particle does not comprise PEG.

4. The nucleic acid-lipid particle of claim 3, wherein the nucleic acid-lipid particle is a component of a multi-dose therapy.

5. The nucleic acid-lipid particle of claim 1 comprising one or more non-cationic lipids comprising from 20 mol % to 80 mol % of the total lipid present in the lipid-nucleic acid particle, optionally wherein the one or more non-cationic lipids comprise cholesterol or a derivative thereof.

6. The nucleic acid-lipid particle of claim 5 comprising cholesterol or a derivative thereof at a concentration range selected from the group consisting of 10 mol % to 20 mol % of the total lipid present in the nucleic acid-lipid particle, 35 mol % to 45 mol % of the total lipid present in the nucleic acid-lipid particle, and 60 mol % to 70 mol % of the total lipid present in the nucleic acid-lipid particle.

7. The nucleic acid-lipid particle of claim 1 comprising one or more non-cationic lipid other than cholesterol or a derivative thereof, optionally wherein the one or more non-cationic lipid other than cholesterol or a derivative thereof comprises from 5 mol % to 20 mol % of the total lipid present in the lipid-nucleic acid particle, optionally wherein the one or more non-cationic lipid other than cholesterol or a derivative thereof comprises about 10 mol % of the total lipid present in the nucleic acid-lipid particle.

8. The nucleic acid-lipid particle of claim 7, wherein the one or more non-cationic lipid other than cholesterol or a derivative thereof comprises a non-cationic lipid selected from the group consisting of 1,2-dioleoyl-sn-glycero-3-phosphocholine (DOPC), 1,2-Distearoyl-sn-glycero-3-phosphocholine (DSPC), 1,2-Dioleoyl-sn-glycero-3-phosphoethanolamine (DOPE) and β-sitosterol, optionally wherein the one or more non-cationic lipid other than cholesterol or a derivative thereof is dioleoylphosphatidylcholine (DOPC).

9. The nucleic acid-lipid particle of claim 1, wherein the nucleic acid cargo comprises a synthetic or naturally occurring RNA or DNA, or derivatives thereof, optionally wherein the nucleic acid cargo is a modified RNA, optionally wherein the modified RNA is selected from the group consisting of a modified mRNA, a modified antisense oligonucleotide and a modified siRNA, optionally wherein the modified mRNA encodes a nucleic acid modulating controller.

10. The nucleic acid-lipid particle of claim 1, wherein the nucleic acid cargo comprises one or more modifications selected from the group consisting of 2′-O-methyl modified nucleotides, a nucleotide comprising a 5′-phosphorothioate group, a terminal nucleotide linked to a cholesteryl derivative, a 2′-deoxy-2′-fluoro modified nucleotide, a 5′-methoxy-modified nucleotide (e.g., 5′-methoxyuridine), a 2′-deoxy-modified nucleotide, a locked nucleotide, an abasic nucleotide, a 2′-amino-modified nucleotide, a 2′-alkyl-modified nucleotide, a morpholino nucleotide, a phosphoramidate, a non-natural base comprising nucleotide; internucleoside linkages or backbones including phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′.

11. The nucleic acid-lipid particle of claim 1, wherein the lung tissue is selected from the group consisting of epithelium, endothelium, interstitial connective tissue, blood vessel, hematopoietic tissue, lymphoid tissue, and pleura.

12. The nucleic acid-lipid particle of claim 1, wherein the nucleic acid-lipid particle comprises DOTAP comprising from 20 mol % to 49 mol % of the total lipid present in the nucleic acid-lipid particle, optionally wherein the nucleic acid-lipid particle comprises DOTAP comprising about 25 mol % or about 45 mol % of the total lipid present in the nucleic acid-lipid particle.

13. The nucleic acid-lipid particle of claim 1 comprising DOTAP comprising about 50 mol % or about 75 mol % of the total lipid present in the nucleic acid-lipid particle.

14. A composition selected from the following:
a pharmaceutical composition comprising the nucleic acid-lipid particle of claim 1 and a pharmaceutically acceptable carrier;
a polyethylene glycol (PEG)-free lipid-nucleic acid particle for delivering a nucleic acid cargo to a tissue of a subject, the PEG-free lipid-nucleic acid particle comprising 1,2-dioleoyl-3-trimethylammonium-propane (DOTAP) comprising from 20 mol % to 80 mol % of the total lipid present in the PEG-free lipid-nucleic acid particle;
a nucleic acid-lipid particle comprising 1,2-dioleoyl-3-trimethylammonium-propane (DOTAP) at about 45 mol % of the total lipid present in the nucleic acid-lipid particle and having a N/P ratio of about 3;
a nucleic acid-lipid particle comprising 1,2-dioleoyl-3-trimethylammonium-propane (DOTAP) at about 45 mol % of the total lipid present in the nucleic acid-lipid particle and having a N/P ratio of about 6;
a nucleic acid-lipid particle comprising 1,2-dioleoyl-3-trimethylammonium-propane (DOTAP) at about 25 mol % of the total lipid present in the nucleic acid-lipid particle, one or more non-cationic lipid other than cholesterol or a derivative thereof at about 10 mol % of the total lipid present in the nucleic acid-lipid particle and cholesterol or a derivative thereof at about 63 mol % to about 65 mol % of the total lipid present in the nucleic acid-lipid particle;
a nucleic acid-lipid particle comprising 1,2-dioleoyl-3-trimethylammonium-propane (DOTAP) at about 50 mol % of the total lipid present in the nucleic acid-lipid particle, one or more non-cationic lipid other than cholesterol or a derivative thereof at about 10 mol % of the total lipid present in the nucleic acid-lipid particle and cholesterol or a derivative thereof at about 38 mol % to about 40 mol % of the total lipid present in the nucleic acid-lipid particle;
a nucleic acid-lipid particle comprising 1,2-dioleoyl-3-trimethylammonium-propane (DOTAP) at about 75 mol % of the total lipid present in the nucleic acid-lipid particle, one or more non-cationic lipid other than cholesterol or a derivative thereof at about 10 mol % of the total lipid present in the nucleic acid-lipid particle and cholesterol or a derivative thereof at about 13 mol % to about 15 mol % of the total lipid present in the nucleic acid-lipid particle; or
an injectate comprising the nucleic acid-lipid particle of claim 1.

15. The pharmaceutical composition, PEG-free lipid-nucleic acid particle, nucleic acid-lipid particle, or injectate of claim 14, wherein:
the pharmaceutical composition is formulated for parenteral administration, optionally for intravenous injection;
the pharmaceutical composition is formulated for inhalation;
the pharmaceutical composition is formulated for direct injection into the lung tissue;
intravenous administration of the nucleic acid-lipid particle or pharmaceutical composition to the subject results in expression of the nucleic acid cargo in cells of the lung tissue of the subject at a level that is at least two-fold higher than expression of the nucleic acid cargo in cells of liver, heart, spleen, ovary, pancreas and kidney of the subject, optionally wherein expression of the nucleic acid cargo in cells of the lung tissue of the subject is at least three-fold higher, optionally at least four-fold higher, optionally at least five-fold higher, optionally at least six-fold higher, optionally at least seven-fold higher, optionally at least eight-fold higher, optionally at least nine-fold higher, optionally at least ten-fold higher, optionally at least eleven-fold higher, optionally at least twelve-fold higher, optionally at least thirteen-fold higher, optionally at least fourteen-fold higher, optionally at least fifteen-fold higher, optionally at least twenty-fold higher, than expression of the nucleic acid cargo in cells of liver, heart, spleen, ovary, pancreas and kidney of the subject;
intravenous administration of the nucleic acid-lipid particle or pharmaceutical composition to the subject results in localization of the nucleic acid-lipid particle to the lung tissue of the subject at an at least two-fold higher concentration than the concentration of the nucleic acid-lipid particle in one or more other tissues of the subject selected from the group consisting of heart, spleen, ovaries and pancreas, optionally wherein at least three-fold, optionally at least four-fold, optionally at least five-fold, optionally at least six-fold higher concentration of the nucleic acid-lipid particle is located in lung as compared to one or more other tissues of the subject selected from the group consisting of heart, spleen, ovaries and pancreas;
the nucleic acid-lipid particle or pharmaceutical composition is administered to treat a lung disease or disorder, optionally wherein the disease or disorder is selected from the group consisting of lung cancer, pneumonia, pulmonary fibrosis, COPD, asthma, bronchiectasis, sarcoidosis, pulmonary hypertension, emphysema, alpha-1 antitrypsin deficiency, aspergillosis, bronchiolitis, bronchitis, pneumoconiosis, a coronavirus, Middle Eastern Respiratory Syndrome, Severe Acute Respiratory Syndrome, cystic fibrosis, Legionnaire's disease, influenza, pertussis, pulmonary embolism and tuberculosis;
the PEG-free lipid-nucleic acid particle comprises one or more non-cationic lipids comprising from 20 mol % to 80 mol % of the total lipid present in the PEG-free lipid-nucleic acid particle, optionally wherein the non-cationic lipid comprises cholesterol or a derivative thereof;
the PEG-free lipid-nucleic acid particle comprises cholesterol or a derivative thereof at a concentration range selected from the group consisting of 10 mol % to 20 mol % of the total lipid present in the PEG-free lipid-nucleic acid particle, 35 mol % to 45 mol % of the total lipid present in the PEG-free lipid-nucleic acid particle, and 60 mol % to 70 mol % of the total lipid present in the PEG-free lipid-nucleic acid particle;
the PEG-free lipid-nucleic acid particle comprises one or more non-cationic lipid other than cholesterol or a derivative thereof, optionally wherein the one or more non-cationic lipid other than cholesterol or a derivative thereof comprises from 5 mol % to 20 mol % of the total lipid present in the PEG-free lipid-nucleic acid particle, optionally wherein the one or more non-cationic lipid other than cholesterol or a derivative thereof comprises about 10 mol % of the total lipid present in the PEG-free lipid-nucleic acid particle;
the one or more non-cationic lipid other than cholesterol or a derivative thereof comprises a non-cationic lipid selected from the group consisting of 1,2-dioleoyl-sn-glycero-3-phosphocholine (DOPC), 1,2-Distearoyl-sn-glycero-3-phosphocholine (DSPC), 1,2-Dioleoyl-sn-glycero-3-phosphoethanolamine (DOPE) and β-sitosterol, optionally wherein the one or more non-cationic lipid other than cholesterol or a derivative thereof is dioleoylphosphatidylcholine (DOPC);
the nucleic acid cargo comprises a synthetic or naturally occurring RNA or DNA, or derivatives thereof, optionally wherein the nucleic acid cargo is a modified RNA, optionally wherein the RNA is selected from the group consisting of a mRNA, an antisense oligonucleotide and a siRNA, optionally wherein the mRNA encodes a nucleic acid modulating controller (i.e., a mRNA that encodes for protein controller components);
the nucleic acid cargo comprises a modification selected from the group consisting of 2′-O-methyl modified nucleotides, a nucleotide comprising a 5′-phosphorothioate group, a terminal nucleotide linked to a cholesteryl derivative, a 2′-deoxy-2′-fluoro modified nucleotide, a 5′-methoxy-modified nucleotide (e.g., 5′-methoxyuridine), a 2′-deoxy-modified nucleotide, a locked nucleotide, an abasic nucleotide, a 2′-amino-modified nucleotide, a 2′-alkyl-modified nucleotide, a morpholino nucleotide, a phosphoramidate, a non-natural base comprising nucleotide;
internucleoside linkages or backbones including phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′;
the tissue is one or more selected from the group consisting of lung, joint, epidermis, dermis, endothelium, and blood tissues;
the PEG-free nucleic acid-lipid particle is administered parenterally, optionally wherein the PEG-free nucleic acid-lipid particle is administered via a route selected from the group consisting of inhalation, topical application and injection, optionally wherein the injection is selected from the group consisting of intravenous injection, intratracheal injection or instillation, intra-articular injection, subcutaneous injection, intradermal injection and intramuscular injection; and/or
the PEG-free nucleic acid-lipid particle is a component of a multi-dose therapy.

16-30. (canceled)

31. A pharmaceutical composition comprising the composition of claim 14 and a pharmaceutically acceptable carrier.

32. The pharmaceutical composition of claim 31, wherein:
the pharmaceutical composition is formulated for parenteral administration, optionally for intravenous injection;
the pharmaceutical composition is formulated for inhalation;
the pharmaceutical composition is formulated for direct injection into the tissue;
the pharmaceutical composition is administered to a tissue selected from the group consisting of lung, joint, epidermis, dermis, endothelium and blood tissue;
the pharmaceutical composition is administered to the subject to treat or prevent a disease or disorder selected from the group consisting of: a lung disease or disorder, optionally wherein the lung disease or disorder is selected from the group consisting of lung cancer, pneumonia, pulmonary fibrosis, chronic obstructive pulmonary disease (COPD), asthma, bronchiectasis, sarcoidosis, pulmonary hypertension, emphysema, alpha-1 antitrypsin deficiency, aspergillosis, bronchiolitis, bronchitis, pneumoconiosis, a coronavirus (e.g., SARS-CoV-2), Middle Eastern Respiratory Syndrome, Severe Acute Respiratory Syndrome, cystic fibrosis, Legionnaire's disease, influenza, pertussis, pulmonary embolism and tuberculosis: a joint disease or disorder, optionally wherein the joint disease or disorder is selected from the group consisting of rheumatoid arthritis, psoriatic arthritis, gout, tendinitis, bursitis, Carpal Tunnel Syndrome and osteoarthritis; an inflammatory disease or disorder, optionally wherein the inflammatory disease or disorder is selected from the group consisting of inflammatory bowel disease, peritonitis, osteomyelitis, cachexia, pancreatitis, trauma induced shock, bronchial asthma, allergic rhinitis, cystic fibrosis, acute bronchitis, acute intense bronchitis, osteoarthritis, rheumatoid arthritis, infectious arthritis, post-infectious arthritis, gonocoele arthritis, tuberculous arthritis, arthritis, osteoarthritis, gout, spondyloarthropathies, ankylosing spondylitis, arthritis associated with vasculitis syndrome, nodular polyarteritis nervosa, irritable vasculitis, rugenic granulomatosis, rheumatoid polyposis myalgia, arthritis cell arteritis, calcium polycystic arthropathy, caustic gout, non-arthritic rheumatism, bursitis, hay fever, suppurative inflammation (e.g., tennis elbow), neuropathic joint disease, hemarthrosic, Henoch-Schlein purpura, hypertrophic osteoarthritis, multisized hemorrhoids, scoliosis, hemochromatosis, hyperlipoproteinemia, hypogammaglobulinemia, COPD, acute respiratory distress syndrome, acute lung injury, broncho-pulmonary dysplasia and systemic lupus erythematosus (SLE); and an epidermal disease or disorder, optionally wherein the epidermal disease or disorder is selected from the group consisting of psoriasis, atopic dermatitis, scleroderma, eczema, rosacea, seborrheic dermatitis, melanoma, solar keratosis, ichthyosis, Grover's disease, common warts, keratoacanthoma and seborrhoeic keratosis;
the nucleic acid-lipid particle does not comprise a PEG-lipid conjugate, optionally wherein the nucleic acid-lipid particle does not comprise PEG;
the nucleic acid-lipid particle comprises a conjugated lipid that inhibits aggregation of particles comprising about 1.0% of the total lipid present, optionally wherein the conjugated lipid comprises a polyethyleneglycol (PEG)-lipid conjugate, optionally wherein the PEG of the PEG-lipid conjugate has an average molecular weight of from 550 daltons to 3000 daltons, optionally wherein the PEG-lipid conjugate is a PEG2000-lipid conjugate, optionally wherein the PEG2000-lipid conjugate comprises one or more of 1,2-dimyristoyl-rac-glycero-3-methoxypolyethylene glycol-2000 (DMG-PEG2k) and 1,2-distearoyl-rac-glycero-3-methoxypolyethylene glycol-2000 (DSG-PEG2k), optionally wherein the PEG2000-lipid conjugate is 1,2-Dimyristoyl-rac-glycero-3-methoxypolyethylene glycol-2000 (DMG-PEG2k), optionally wherein the nucleic acid-lipid particle comprises a PEG-lipid conjugate at a concentration of about 1.0 mol % of the total lipid present in the nucleic acid-lipid particle, optionally wherein the nucleic acid-lipid particle comprises a conjugated lipid that inhibits aggregation of particles comprising about 1.0% of the total lipid present, optionally wherein the conjugated lipid comprises a polyethyleneglycol (PEG)-lipid conjugate, optionally wherein the PEG of the PEG-lipid conjugate has an average molecular weight of from 550 daltons to 3000 daltons, optionally wherein the PEG-lipid conjugate is a PEG2000-lipid conjugate, optionally wherein the PEG2000-lipid conjugate comprises one or more of 1,2-dimyristoyl-rac-glycero-3-methoxypolyethylene glycol-2000 (DMG-PEG2k) and 1,2-distearoyl-rac-glycero-3-methoxypolyethylene glycol-2000 (DSG-PEG2k), optionally wherein the PEG2000-lipid conjugate is 1,2-Dimyristoyl-rac-glycero-3-methoxypolyethylene glycol-2000 (DMG-PEG2k), optionally wherein the nucleic acid-lipid particle comprises a PEG-lipid conjugate at a concentration of about 1.0 mol % of the total lipid present in the nucleic acid-lipid particle, or optionally wherein the nucleic acid-lipid particle comprises a conjugated lipid that inhibits aggregation of particles comprising about 2.0% of the total lipid present, optionally wherein the conjugated lipid comprises a polyethyleneglycol (PEG)-lipid conjugate, optionally wherein the PEG of the PEG-lipid conjugate has an average molecular weight of from 550 daltons to 3000 daltons, optionally wherein the PEG-lipid conjugate is a PEG2000-lipid conjugate, optionally wherein the PEG2000-lipid conjugate comprises one or more of 1,2-dimyristoyl-rac-glycero-3-methoxypolyethylene glycol-2000 (DMG-PEG2k) and 1,2-distearoyl-rac-glycero-3-methoxypolyethylene glycol-2000 (DSG-PEG2k), optionally wherein the PEG2000-lipid conjugate is 1,2-Dimyristoyl-rac-glycero-3-methoxypolyethylene glycol-2000 (DMG-PEG2k), optionally wherein the nucleic acid-lipid particle comprises a PEG-lipid conjugate at a concentration of about 2.0 mol % of the total lipid present in the nucleic acid-lipid particle;
the nucleic acid-lipid particle does not comprise a PEG-lipid conjugate, optionally wherein the nucleic acid-lipid particle comprises cholesterol or a derivative thereof at about 39.75 mol % of the total lipid present in the nucleic acid-lipid particle, optionally wherein the N/P ratio of the nucleic acid-lipid particle is about 2;
the nucleic acid-lipid particle does not comprise a PEG-lipid conjugate, optionally wherein the nucleic acid-lipid particle comprises cholesterol or a derivative thereof at about 39.75 mol % of the total lipid present in the nucleic acid-lipid particle, optionally wherein the N/P ratio of the nucleic acid-lipid particle is about 3;
the nucleic acid-lipid particle comprises a conjugated lipid that inhibits aggregation of particles comprising about 0.5% of the total lipid present, optionally wherein the conjugated lipid comprises a polyethyleneglycol (PEG)-lipid conjugate, optionally wherein the PEG of the PEG-lipid conjugate has an average molecular weight of from 550 daltons to 3000 daltons, optionally wherein the PEG-lipid conjugate is a PEG2000-lipid conjugate, optionally wherein the PEG2000-lipid conjugate comprises one or more of 1,2-dimyristoyl-rac-glycero-3-methoxypolyethylene glycol-2000 (DMG-PEG2k) and 1,2-distearoyl-rac-glycero-3-methoxypolyethylene glycol-2000 (DSG-PEG2k), optionally wherein the PEG2000-lipid conjugate is 1,2-Dimyristoyl-rac-glycero-3-methoxypolyethylene glycol-2000 (DMG-PEG2k), optionally wherein the nucleic acid-lipid particle comprises a PEG-lipid conjugate at about 0.5% of the total lipid present in the nucleic acid-lipid particle, optionally wherein the nucleic acid-lipid particle comprises cholesterol or a derivative thereof at about 39.25 mol % of the total lipid present in the nucleic acid-lipid particle, optionally wherein the N/P ratio of the nucleic acid-lipid particle is about 3;
the nucleic acid-lipid particle comprises a conjugated lipid that inhibits aggregation of particles comprising about 1.0% of the total lipid present, optionally wherein the conjugated lipid comprises a polyethyleneglycol (PEG)-lipid conjugate, optionally wherein the PEG of the PEG-lipid conjugate has an average molecular weight of from 550 daltons to 3000 daltons, optionally wherein the PEG-lipid conjugate is a PEG2000-lipid conjugate, optionally wherein the PEG2000-lipid conjugate comprises one or more of 1,2-dimyristoyl-rac-glycero-3-methoxypolyethylene glycol-2000 (DMG-PEG2k) and 1,2-distearoyl-rac-glycero-3-methoxypolyethylene glycol-2000 (DSG-PEG2k), optionally wherein the PEG2000-lipid conjugate is 1,2-Dimyristoyl-rac-glycero-3-methoxypolyethylene glycol-2000 (DMG-PEG2k), optionally wherein the nucleic acid-lipid particle comprises a PEG-lipid conjugate at about 1.0% of the total lipid present in the nucleic acid-lipid particle, optionally wherein the nucleic acid-lipid particle comprises cholesterol or a derivative thereof at about 38.75 mol % of the total lipid present in the nucleic acid-lipid particle, optionally wherein the N/P ratio of the nucleic acid-lipid particle is about 3;
the nucleic acid-lipid particle comprises a conjugated lipid that inhibits aggregation of particles comprising about 1.0% of the total lipid present, optionally wherein the conjugated lipid comprises a polyethyleneglycol (PEG)-lipid conjugate, optionally wherein the PEG of the PEG-lipid conjugate has an average molecular weight of from 550 daltons to 3000 daltons, optionally wherein the PEG-lipid conjugate is a PEG2000-lipid conjugate, optionally wherein the PEG2000-lipid conjugate comprises one or more of 1,2-dimyristoyl-rac-glycero-3-methoxypolyethylene glycol-2000 (DMG-PEG2k) and 1,2-distearoyl-rac-glycero-3-methoxypolyethylene glycol-2000 (DSG-PEG2k), optionally wherein the PEG2000-lipid conjugate is 1,2-Dimyristoyl-rac-glycero-3-methoxypolyethylene glycol-2000 (DMG-PEG2k), optionally wherein the nucleic acid-lipid particle comprises a PEG-lipid conjugate at about 1.0% of the total lipid present in the nucleic acid-lipid particle, optionally wherein the nucleic acid-lipid particle comprises cholesterol or a derivative thereof at about 38.75 mol % of the total lipid present in the nucleic acid-lipid particle, optionally wherein the N/P ratio of the nucleic acid-lipid particle is about 2;
the nucleic acid-lipid particle comprises a conjugated lipid that inhibits aggregation of particles comprising about 1.5% of the total lipid present, optionally wherein the conjugated lipid comprises a polyethyleneglycol (PEG)-lipid conjugate, optionally wherein the PEG of the PEG-lipid conjugate has an average molecular weight of from 550 daltons to 3000 daltons, optionally wherein the PEG-lipid conjugate is a PEG2000-lipid conjugate, optionally wherein the PEG2000-lipid conjugate comprises one or more of 1,2-dimyristoyl-rac-glycero-3-methoxypolyethylene glycol-2000 (DMG-PEG2k) and 1,2-distearoyl-rac-glycero-3-methoxypolyethylene glycol-2000 (DSG-PEG2k), optionally wherein the PEG2000-lipid conjugate is 1,2-Dimyristoyl-rac-glycero-3-methoxypolyethylene glycol-2000 (DMG-PEG2k), optionally wherein the nucleic acid-lipid particle comprises a PEG-lipid conjugate at about 1.5% of the total lipid present in the nucleic acid-lipid particle, optionally wherein the nucleic acid-lipid particle comprises cholesterol or a derivative thereof at about 38.25 mol % of the total lipid present in the nucleic acid-lipid particle, optionally wherein the N/P ratio of the nucleic acid-lipid particle is about 4;
the nucleic acid-lipid particle does not comprise a PEG-lipid conjugate, optionally wherein the nucleic acid-lipid particle comprises cholesterol or a derivative thereof at about 64.75 mol % of the total lipid present in the nucleic acid-lipid particle, optionally wherein the N/P ratio of the nucleic acid-lipid particle is about 3;
the nucleic acid-lipid particle comprises a conjugated lipid that inhibits aggregation of particles comprising about 0.5% of the total lipid present, optionally wherein the conjugated lipid comprises a polyethyleneglycol (PEG)-lipid conjugate, optionally wherein the PEG of the PEG-lipid conjugate has an average molecular weight of from 550 daltons to 3000 daltons, optionally wherein the PEG-lipid conjugate is a PEG2000-lipid conjugate, optionally wherein the PEG2000-lipid conjugate comprises one or more of 1,2-dimyristoyl-rac-glycero-3-methoxypolyethylene glycol-2000 (DMG-PEG2k) and 1,2-distearoyl-rac-glycero-3-methoxypolyethylene glycol-2000 (DSG-PEG2k), optionally wherein the PEG2000-lipid conjugate is 1,2-Dimyristoyl-rac-glycero-3-methoxypolyethylene glycol-2000 (DMG-PEG2k), optionally wherein the nucleic acid-lipid particle comprises a PEG-lipid conjugate at about 0.5% of the total lipid present in the nucleic acid-lipid particle, optionally wherein the nucleic acid-lipid particle comprises cholesterol or a derivative thereof at about 64.25 mol % of the total lipid present in the nucleic acid-lipid particle, optionally wherein the N/P ratio of the nucleic acid-lipid particle is about 4;
the nucleic acid-lipid particle comprises a conjugated lipid that inhibits aggregation of particles comprising about 1.0% of the total lipid present, optionally wherein the conjugated lipid comprises a polyethyleneglycol (PEG)-lipid conjugate, optionally wherein the PEG of the PEG-lipid conjugate has an average molecular weight of from 550 daltons to 3000 daltons, optionally wherein the PEG-lipid conjugate is a PEG2000-lipid conjugate, optionally wherein the PEG2000-lipid conjugate comprises one or more of 1,2-dimyristoyl-rac-glycero-3-methoxypolyethylene glycol-2000 (DMG-PEG2k) and 1,2-distearoyl-rac-glycero-3-methoxypolyethylene glycol-2000 (DSG-PEG2k), optionally wherein the PEG2000-lipid conjugate is 1,2-Dimyristoyl-rac-glycero-3-methoxypolyethylene glycol-2000 (DMG-PEG2k), optionally wherein the nucleic acid-lipid particle comprises a PEG-lipid conjugate at about 1.0% of the total lipid present in the nucleic acid-lipid particle, optionally wherein the nucleic acid-lipid particle comprises cholesterol or a derivative thereof at about 63.75 mol % of the total lipid present in the nucleic acid-lipid particle, optionally wherein the N/P ratio of the nucleic acid-lipid particle is about 4;
the nucleic acid-lipid particle comprises a conjugated lipid that inhibits aggregation of particles comprising about 1.5% of the total lipid present, optionally wherein the conjugated lipid comprises a polyethyleneglycol (PEG)-lipid conjugate, optionally wherein the PEG of the PEG-lipid conjugate has an average molecular weight of from 550 daltons to 3000 daltons, optionally wherein the PEG-lipid conjugate is a PEG2000-lipid conjugate, optionally wherein the PEG2000-lipid conjugate comprises one or more of 1,2-dimyristoyl-rac-glycero-3-methoxypolyethylene glycol-2000 (DMG-PEG2k) and 1,2-distearoyl-rac-glycero-3-methoxypolyethylene glycol-2000 (DSG-PEG2k), optionally wherein the PEG2000-lipid conjugate is 1,2-Dimyristoyl-rac-glycero-3-methoxypolyethylene glycol-2000 (DMG-PEG2k), optionally wherein the nucleic acid-lipid particle comprises a PEG-lipid conjugate at about 1.5% of the total lipid present in the nucleic acid-lipid particle, optionally wherein the nucleic acid-lipid particle comprises cholesterol or a derivative thereof at about 63.25 mol % of the total lipid present in the nucleic acid-lipid particle, optionally wherein the N/P ratio of the nucleic acid-lipid particle is about 2;
the nucleic acid-lipid particle does not comprise a PEG-lipid conjugate, optionally wherein the nucleic acid-lipid particle comprises cholesterol or a derivative thereof at about 14.75 mol % of the total lipid present in the nucleic acid-lipid particle, optionally wherein the N/P ratio of the nucleic acid-lipid particle is about 4;
the nucleic acid-lipid particle comprises a conjugated lipid that inhibits aggregation of particles comprising about 0.5% of the total lipid present, optionally wherein the conjugated lipid comprises a polyethyleneglycol (PEG)-lipid conjugate, optionally wherein the PEG of the PEG-lipid conjugate has an average molecular weight of from 550 daltons to 3000 daltons, optionally wherein the PEG-lipid conjugate is a PEG2000-lipid conjugate, optionally wherein the PEG2000-lipid conjugate comprises one or more of 1,2-dimyristoyl-rac-glycero-3-methoxypolyethylene glycol-2000 (DMG-PEG2k) and 1,2-distearoyl-rac-glycero-3-methoxypolyethylene glycol-2000 (DSG-PEG2k), optionally wherein the PEG2000-lipid conjugate is 1,2-Dimyristoyl-rac-glycero-3-methoxypolyethylene glycol-2000 (DMG-PEG2k), optionally wherein the nucleic acid-lipid particle comprises a PEG-lipid conjugate at about 0.5% of the total lipid present in the nucleic acid-lipid particle, optionally wherein the nucleic acid-lipid particle comprises cholesterol or a derivative thereof at about 14.25 mol % of the total lipid present in the nucleic acid-lipid particle, optionally wherein the N/P ratio of the nucleic acid-lipid particle is about 2;
the nucleic acid-lipid particle comprises a conjugated lipid that inhibits aggregation of particles comprising about 1.0% of the total lipid present, optionally wherein the conjugated lipid comprises a polyethyleneglycol (PEG)-lipid conjugate, optionally wherein the PEG of the PEG-lipid conjugate has an average molecular weight of from 550 daltons to 3000 daltons, optionally wherein the PEG-lipid conjugate is a PEG2000-lipid conjugate, optionally wherein the PEG2000-lipid conjugate comprises one or more of 1,2-dimyristoyl-rac-glycero-3-methoxypolyethylene glycol-2000 (DMG-PEG2k) and 1,2-distearoyl-rac-glycero-3-methoxypolyethylene glycol-2000 (DSG-PEG2k), optionally wherein the PEG2000-lipid conjugate is 1,2-Dimyristoyl-rac-glycero-3-methoxypolyethylene glycol-2000 (DMG-PEG2k), optionally wherein the nucleic acid-lipid particle comprises a PEG-lipid conjugate at about 1.0% of the total lipid present in the nucleic acid-lipid particle, optionally wherein the nucleic acid-lipid particle comprises cholesterol or a derivative thereof at about 13.75 mol % of the total lipid present in the nucleic acid-lipid particle, optionally wherein the N/P ratio of the nucleic acid-lipid particle is about 2;
the nucleic acid-lipid particle comprises a conjugated lipid that inhibits aggregation of particles comprising about 1.5% of the total lipid present, optionally wherein the conjugated lipid comprises a polyethyleneglycol (PEG)-lipid conjugate, optionally wherein the PEG of the PEG-lipid conjugate has an average molecular weight of from 550 daltons to 3000 daltons, optionally wherein the PEG-lipid conjugate is a PEG2000-lipid conjugate, optionally wherein the PEG2000-lipid conjugate comprises one or more of 1,2-dimyristoyl-rac-glycero-3-methoxypolyethylene glycol-2000 (DMG-PEG2k) and 1,2-distearoyl-rac-glycero-3-methoxypolyethylene glycol-2000 (DSG-PEG2k), optionally wherein the PEG2000-lipid conjugate is 1,2-Dimyristoyl-rac-glycero-3-methoxypolyethylene glycol-2000 (DMG-PEG2k), optionally wherein the nucleic acid-lipid particle comprises a PEG-lipid conjugate at about 1.5% of the total lipid present in the nucleic acid-lipid particle, optionally wherein the nucleic acid-lipid particle comprises cholesterol or a derivative thereof at about 13.25 mol % of the total lipid present in the nucleic acid-lipid particle, optionally wherein the N/P ratio of the nucleic acid-lipid particle is about 3; and/or
the one or more non-cationic lipid other than cholesterol or a derivative thereof comprises a non-cationic lipid selected from the group consisting of 1,2-dioleoyl-sn-glycero-3-phosphocholine (DOPC), 1,2-Distearoyl-sn-glycero-3-phosphocholine (DSPC), 1,2-Dioleoyl-sn-glycero-3-phosphoethanolamine (DOPE) and β-sitosterol, optionally wherein the one or more non-cationic lipid other than cholesterol or a derivative thereof is dioleoylphosphatidylcholine (DOPC).

33-67. (canceled)

68. A method for delivering a nucleic acid cargo to a lung tissue of a subject comprising administering to the subject a nucleic acid-lipid particle comprising 1,2-dioleoyl-3-trimethylammonium-propane (DOTAP) comprising from 20 mol % to 80 mol % of the total lipid present in the nucleic acid-lipid particle.

69. A method for treating or preventing a disease or disorder in a subject, the method comprising administering the nucleic acid-lipid particle, pharmaceutical composition, PEG-free lipid-nucleic acid particle or injectate of claim 1 to the subject.

70. The method of claim 69, wherein:
the nucleic acid-lipid particle, pharmaceutical composition, PEG-free lipid-nucleic acid particle or injectate is administered intravenously and expression of the nucleic acid cargo in cells of the lung tissue of the subject occurs at a level that is at least two-fold higher than expression of the nucleic acid cargo in cells of liver, heart, spleen, ovary, pancreas and kidney of the subject, optionally wherein expression of the nucleic acid cargo in cells of the lung tissue of the subject is at least three-fold higher, optionally at least four-fold higher, optionally at least five-fold higher, optionally at least six-fold higher, optionally at least seven-fold higher, optionally at least eight-fold higher, optionally at least nine-fold higher, optionally at least ten-fold higher, optionally at least eleven-fold higher, optionally at least twelve-fold higher, optionally at least thirteen-fold higher, optionally at least fourteen-fold higher, optionally at least fifteen-fold higher, optionally at least twenty-fold higher, than expression of the nucleic acid cargo in cells of liver, heart, spleen, ovary, pancreas and kidney of the subject;
the nucleic acid-lipid particle, pharmaceutical composition, PEG-free lipid-nucleic acid particle or injectate is administered intravenously and the nucleic acid-lipid particle or PEG-free lipid-nucleic acid particle localizes to the lung tissue of the subject at an at least two-fold higher concentration than the concentration of the nucleic acid-lipid particle or PEG-free lipid-nucleic acid particle in one or more other tissues of the subject selected from the group consisting of heart, spleen, ovaries and pancreas, optionally wherein at least three-fold, optionally at least four-fold, optionally at least five-fold, optionally at least six-fold higher concentration of the nucleic acid-lipid particle or PEG-free lipid-nucleic acid particle is present in lung as compared to one or more other tissues of the subject selected from the group consisting of heart, spleen, ovaries and pancreas;
the disease or disorder is selected from the group consisting of: a lung disease or disorder, optionally wherein the lung disease or disorder is selected from the group consisting of lung cancer, pneumonia, pulmonary fibrosis, chronic obstructive pulmonary disease (COPD), asthma, bronchiectasis, sarcoidosis, pulmonary hypertension, emphysema, alpha-1 antitrypsin deficiency, aspergillosis, bronchiolitis, bronchitis, pneumoconiosis, a coronavirus (e.g., SARS-CoV-2), Middle Eastern Respiratory Syndrome, Severe Acute Respiratory Syndrome, cystic fibrosis, Legionnaire's disease, influenza, pertussis, pulmonary embolism and tuberculosis; a joint disease or disorder, optionally wherein the joint disease or disorder is selected from the group consisting of rheumatoid arthritis, psoriatic arthritis, gout, tendinitis, bursitis, Carpal Tunnel Syndrome and osteoarthritis; an inflammatory disease or disorder, optionally wherein the inflammatory disease or disorder is selected from the group consisting of inflammatory bowel disease, peritonitis, osteomyelitis, cachexia, pancreatitis, trauma induced shock, bronchial asthma, allergic rhinitis, cystic fibrosis, acute bronchitis, acute intense bronchitis, osteoarthritis, rheumatoid arthritis, infectious arthritis, post-infectious arthritis, gonocoele arthritis, tuberculous arthritis, arthritis, osteoarthritis, gout, spondyloarthropathies, ankylosing spondylitis, arthritis associated with vasculitis syndrome, nodular polyarteritis nervosa, irritable vasculitis, rugenic granulomatosis, rheumatoid polyposis myalgia, arthritis cell arteritis, calcium polycystic arthropathy, caustic gout, non-arthritic rheumatism, bursitis, hay fever, suppurative inflammation (e.g., tennis elbow), neuropathic joint disease, hemarthrosic, Henoch-Schlein purpura, hypertrophic osteoarthritis, multisized hemorrhoids, scoliosis, hemochromatosis, hyperlipoproteinemia, hypogammaglobulinemia, COPD, acute respiratory distress syndrome, acute lung injury, broncho-pulmonary dysplasia and systemic lupus erythematosus (SLE); and an epidermal disease or disorder, optionally wherein the epidermal disease or disorder is selected from the group consisting of psoriasis, atopic dermatitis, scleroderma, eczema, rosacea, seborrheic dermatitis, melanoma, solar keratosis, ichthyosis, Grover's disease, common warts, keratoacanthoma and seborrhoeic keratosis;
the nucleic acid-lipid particle, pharmaceutical composition, PEG-free lipid-nucleic acid particle or injectate is administered parenterally, optionally wherein the nucleic acid-lipid particle, pharmaceutical composition, PEG-free lipid-nucleic acid particle or injectate is administered via a route selected from the group consisting of inhalation, topical application and injection, optionally wherein the injection is selected from the group consisting of intravenous injection, intratracheal injection or instillation, intra-articular injection, subcutaneous injection, intradermal injection and intramuscular injection;
the nucleic acid cargo comprises a synthetic or naturally occurring RNA or DNA, or derivatives thereof, optionally wherein the nucleic acid cargo is a modified RNA, optionally wherein the modified RNA is selected from the group consisting of a modified mRNA, a modified antisense oligonucleotide and a modified siRNA, optionally wherein the modified mRNA encodes a nucleic acid modulating controller; and/or
the nucleic acid cargo comprises one or more modifications selected from the group consisting of 2′-O-methyl modified nucleotides, a nucleotide comprising a 5′-phosphorothioate group, a terminal nucleotide linked to a cholesteryl derivative, a 2′-deoxy-2′-fluoro modified nucleotide, a 5′-methoxy-modified nucleotide (e.g., 5′-methoxyuridine), a 2′-deoxy-modified nucleotide, a locked nucleotide, an abasic nucleotide, a 2′-amino-modified nucleotide, a 2′-alkyl-modified nucleotide, a morpholino nucleotide, a phosphoramidate, a non-natural base comprising nucleotide: internucleoside linkages or backbones including phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′.

71-75. (canceled)
